(19) United States
(12) Patent Application Publication
(10) Pub. No.: US 2010/0058490 A1
(43) Pub. Date: Mar. 4, 2010

(54) Gene Silencing Methods

(75) Inventors: Peter Waterhouse, Australian Capital Territory (AU); Ming-Bo Wang, Australian Capital Territory (AU)

Correspondence Address: BUCHANAN, INGERSOLL & ROONEY PC, POST OFFICE BOX 1404, ALEXANDRIA, VA, 22313-1404, US

(73) Assignee: COMMONWEALTH SCHIENTIFIC AND INDUSTRIAL RESEARCH ORGANIZATION, CAMPBELL (AU)

(21) Appl. No.: 12/226,951

(22) PCT Filed: May 3, 2007
(86) PCT No.: PCT/AU2007/000583

§ 371 Date: Sep. 16, 2009

Related U.S. Application Data
(60) Provisional application No. 60/798,020, filed on May 4, 2006.

(30) Foreign Application Priority Data
May 3, 2006 (EP) 06075995.8

(51) Int. Cl. C12N 15/82; A01H 1/02; C12P 19/34; C12N 5/10; C07H 21/04; C12N 15/87; C12Q 1/02; C12Q 1/68

(52) U.S. Cl. 800/260; 800/278; 435/91.3; 435/419; 536/23.1; 435/325; 435/463; 435/468; 435/29; 435/6

(57) ABSTRACT

Methods and means are provided to modulate gene silencing in eukaryotes through the alteration of the functional level of particular DICER or DICER like proteins. Also provided are methods and means to modulate post-transcriptional gene silencing in eukaryotes through the alteration of the functional level of proteins involved in transcriptional silencing of the silencing RNA encoding genes.

### FIELD OF THE INVENTION

The invention relates to the field of agriculture, more particularly to the modification of plants by genetic engineering. Described are methods for modifying so-called gene silencing in plants or other eukaryotic organisms by modulating the functional level of enzymes with ribonuclease activity responsible for the generation of RNA intermediates in various gene silencing pathways. Also described are methods for modifying gene silencing in plant cells or plants through modification of genes that have an influence on the initiation or maintenance of gene silencing by the silencing RNA encoding chimeric genes, such as genes involved in RNA directed DNA methylation. Thus, methods and means are provided to modulate post-transcriptional gene silencing in eukaryotes through the alteration of the functional level of proteins involved in transcriptional silencing of the silencing RNA encoding genes.

### BACKGROUND TO THE INVENTION

Gene silencing is a common phenomenon in eukaryotes, whereby the expression of particular genes is reduced or even abolished through a number of different mechanisms ranging from mRNA degradation (post transcriptional silencing) over repression of protein synthesis to chromatin remodeling (transcriptional silencing).

The gene-silencing phenomenon has been quickly adopted to engineer the expression of different target molecules. Initially, two predominant methods for the modulation of gene expression in eukaryotic organisms were known, which are referred to in the art as “antisense” downregulation or “sense” downregulation.

In the last decade, it has been demonstrated that the silencing efficiency could be greatly improved both on quantitative and qualitative level using chimeric constructs encoding RNA capable of forming a double stranded RNA by basepairing between the antisense and sense RNA nucleotide sequences respectively complementary and homologous to the target sequences. Such double stranded RNA (dsRNA) is also referred to as hairpin RNA (hpRNA).

The following references describe the use of such methods:

Fire et al., 1998 describe specific genetic interference by experimental introduction of double-stranded RNA in Caenorhabditis elegans.

WO 99/32619 provides a process of introducing an RNA into a living cell to inhibit gene expression of a target gene in that cell. The process may be practiced ex vivo or in vivo. The RNA has a region with double-stranded structure. Inhibition is sequence-specific in that the nucleotide sequences of the duplex region of the RNA and or a portion of the target gene are identical.

Waterhouse et al. 1998 describe that virus resistance and gene silencing in plants can be induced by simultaneous expression of sense and anti-sense RNA. The sense and antisense RNA may be located in one transcript that has self-complementarity.

Hamilton et al. 1998 describes that a transgene with repeated DNA, i.e., inverted copies of its 5′ untranslated region, causes high frequency, post-transcriptional suppression of ACC-oxidase expression in tomato.

WO 98/53083 describes constructs and methods for enhancing the inhibition of a target gene within an organism which involve inserting into the gene silencing vector an inverted repeat sequence of all Or part of a polynucleotide region within the vector.

WO 99/53050 provides methods and means for reducing the phenotypic expression of a nucleic acid of interest in eukaryotic cells, particularly in plant cells, by introducing chimeric genes encoding sense and antisense RNA molecules directed towards the target nucleic add. These molecules are capable of forming a double stranded RNA region by base-pairing between the regions with the sense and antisense nucleotide sequence or by introducing the RNA molecules themselves. Preferably, the RNA molecules comprise simultaneously both sense and antisense nucleotide sequences.

WO 99/49029 relates generally to a method of modifying gene expression and to synthetic genes for modifying endogenous gene expression M a cell, tissue or organ of a transgenic organism, in particular to a transgenic animal or plant. Synthetic genes and genetic constructs, capable of forming a dsRNA which are capable of repressing, delaying or otherwise reducing the expression of an endogenous gene or a target gene in an organism when introduced thereto are also provided.

WO 99/61631 relates to methods to alter the expression of a target gene in a plant using sense and antisense RNA fragments of the gene. The sense and antisense RNA fragments are capable of pairing and forming a double-stranded RNA molecule, thereby altering the expression of the gene. The present invention also relates, to plants, their progeny and seeds thereof obtained using these methods.

WO 00/01846 provides a method of identifying DNA responsible for conferring a particular phenotype in a cell which method comprises a) constructing a cDNA or genomic library of the DNA of the cell in a suitable vector in an orientation relative to (a) promoter(s) capable of initiating transcription of the cDNA or DNA to double stranded (ds) RNA upon binding of an appropriate transcription factor to the promoter(s); b) introducing the library into one or more of cells comprising the transcription factor, and c) identifying and isolating a particular phenotype of a cell comprising the library and identifying the DNA or cDNA fragment from the library responsible for conferring the phenotype. Using this technique, it is also possible to assign function to a known DNA sequence by a) identifying homologues of the DNA sequence in a cell, b) isolating the relevant DNA homologue(s) or a fragment thereof from the cell, c) cloning the homologue or fragment thereof into an appropriate vector in an orientation relative to a suitable promoter capable of initiating transcription of dsRNA from said DNA homologue or fragment upon binding of an appropriate transcription factor to the promoter and d) introducing the vector into the cell from step a) comprising the transcription factor.

, WO 00/44914 also describes composition and methods for in vivo and in vitro attenuation of gene expression using double stranded RNA, particularly in zebrafish.

WO 00/49035 discloses a method for silencing the expression of an endogenous gene in a cell, the method involving overexpressing in the cell a nucleic acid molecule of the endogenous gene and an antisense molecule including a nucleic acid molecule complementary to the nucleic acid molecule of the endogenous gene, wherein the overexpression of the nucleic acid molecule of the endogenous gene and the antisense molecule in the cell silences the expression of the endogenous gene.

Smith et al., 2000 as well as WO 99/53050 described that intron containing dsRNA further increased the efficiency of silencing. Intron containing hairpin RNA is often also referred to as ihpRNA.

Although gene silencing was initially thought of as a consequence of the introduction of aberrant RNA molecules, such as upon the introduction of transgenes (transcribed to antisense sense or double stranded RNA molecules) it has recently become clear that these phenomena are not just experimental artifacts. RNA mediated gene silencing in eukaryotes appears to play an important role in diverse biological processes, such as spatial and temporal regulation of development, heterochromatin formation and antiviral defense.

All eukaryotes possess a mechanism that generates small RNAs which are then used to regulate gene expression at the transcriptional or post-transcriptional level. Various double stranded RNA substrates are processed into small, 21-24 nucleotide long RNA molecules through the action of specific ribonucleases (Dicer or Dicer-Like (DCL) proteins). These small RNAs serve as guide molecules for protein complexes (RNA-induced silencing complexes (RISC)) which lead to the various effects achieved through gene silencing.

Small RNAs involved in repression of gene expression in eukaryotes through sequence specific interactions with RNA or DNA are generally subdivided in two classes: microRNAs (miRNAs) and small interfering RNAs (siRNAs). These classes of small RNA molecules are distinguished by the structure of their precursors and by their targets. miRNAs are cleaved from the short, imperfectly paired stern of a much larger foldback transcript and regulate the expression of transcripts to which they may have limited similarity. siRNAs arise from a long double stranded RNA (dsRNA) and typically direct the cleavage of transcripts to which they are completely complementary, including the transcript from which they are derived (Yoshikawa et al., 2005, Genes & Development, 19: 2164-2175).

The number of Dicer family members varies greatly among organisms. In humans and C. elegans there is only one identified Dicer. In Drosophila, Dicer-1 and Dicer-2 are both required for small interfering RNA directed mRNA cleavage, whereas Dicer-1 but not Dicer-2 is essential for microRNA directed repression (Lee et al., 2004, Pham et al., 2004).

Plants, such as Arabidopsis, appear to have at least four Dicer-like (DCL) proteins and it has been suggested in the scientific literature that these DCLs are functionally specialized (Qi et al., 2005 Molecular Cell, 19, 421-428)

DCL1 processes miRNAs from partially double-stranded stem-loop precursor RNAs transcribed from MIR genes (Kurihara and Watanabe, 2004, Proc. Natl. Acad. Sci. USA 101: 12753-12758).

DCL3 processes endogenous repeat and intergenic-region-derived siRNAs that depend on RNA dependent RNA polymerase 2 and is involved in the accumulation of the 24 nt siRNAs implicated in DNA and histone methylation (Xie at al., 2004, PLosBiology, 2004, 2, 642-652).

DCL2 appears to function in the antiviral silencing response for some, but not all plant-viruses ((Xie et al., 2004, PLosBiology, 2004, 2, 642-652).

Several publications have ascribed a role to DCL4 in the production of trans-acting siRNAs (ta-siRNAs). ta-siRNAs are a special class of endogenous siRNAs encoded by three known families of genes, designated TAS1, TAS2 and TAS3 in Arabidopsis thaliana. The biogenesis pathway for ta-siRNAs involves site-specific cleavage of primary transcripts guided by a miRNA. The processed transcript is then converted to dsRNA through the activities of RDR6 and SGS3. DCL4 activity then catalyzes the conversion of the dsRNA into siRNA duplex formation in 21-nt increments (Xie et al. 2005, Proc. Natl. Acad. Sci. USA 102, 12984-12989; Yoshikawa et al., 2005, Genes & Development, 19: 2164-2175; Gasciolli et al., 2005 Current Biology, 15, 1494-1500). As indicated in Xie et al. 2005 (supra) whether DCL4 is necessary for transgene and antiviral silencing remains to be determined.

Dunoyer et al. 2005 (Nature Genetics, 37 (12) pp 1356 to 1360) describe that DCL4 is required for RNA interference and produces the 21-nucleotide small interfering RNA component of the plant cell-to-cell silencing signal.

WO2004/096995 describes Dicer proteins from guar (Cyamopsis tetragonoloba), corn (Zea mays), rice (Oryza sativa), soybean (Glycine max) and wheat (Triticum aestivum). The patent application also suggests the construction of recombinant DNA constructs encoding all or portion of these Dicer proteins in sense or antisense orientation, wherein expression of the recombinant DNA construct results in production of altered levels of the Dicer in a transformed host cell.

Cao et al. (2003) described the role of the DRM and CMT3 methyltransferases in RNA directed DNA methylation. Neither drm nor cmt3 mutants affected the maintenance of pre-established RNA directed CpG methylation. The methyltransferases were described as appearing to act downstream of the generation of siRNAs, since drm1 drm2 cmt3 triple mutants showed a lack of non-CpG methylation but elevated levels of siRNAs.

None of the prior art documents describe the possibility of modulating the gene-silencing effect achieved by introduction of double stranded RNA molecules or the genes encoding such dsRNA through the modulation of the functional level of particular types of Dicer-like proteins or through the modulation of genes involved in transcriptional silencing of the silencing RNA encoding chimeric genes in plants or other eukaryotic organisms. These and other problems have been solved as hereinafter described in the different embodiment, examples and claims.

### SUMMARY OF THE INVENTION

In one embodiment, the current invention provides the use of a eukaryotic cell or non-human organism with a modified functional level of a Dicer protein, particularly a DCL3 or DCL4 protein, to reduce the expression of a gene of interest, wherein the gene of interest is silenced in said cell by providing said cell with a gene-silencing molecule. If the eukaryotic cell is a cell other than a plant cell, the modified functional level of DCL 3 or DCL4 protein is an increased level of activity, preferably of DCL4 activity.

In another embodiment, the current invention provides the use of a plant or plant cell with a modified functional level of a protein involved in processing of artificially introduced double-stranded RNA (dsRNA) molecules in short interfering RNA (siRNA), preferably a dicer-like protein such as DCL3 or DCL 4, to modulate a gene-silencing effect achieved by the introduction of a gene-silencing chimeric gene. The gene-silencing chimeric gene may be a gene encoding a silencing RNA, the silencing RNA being selected from a sense RNA, an antisense RNA, an unpolyadenylated sense or antisense RNA, a sense or antisense RNA further comprising a largely double stranded region, hairpin RNA (hpRNA) or micro-RNA (miRNA).

In another embodiment, the invention relates to the use of a plant or plant cell with modified functional level of a Dicer-like 3 protein to modulate the gene-silencing effect obtained by introduction of silencing RNA involving a double stranded RNA during the processing of the silencing RNA into siRNA, such as a dsRNA or hpRNA. The modulation of the functional level of the Dicer-like 3 may be a decrease in the functional level, achieved e.g. by mutation of the Dicer-like 3 protein encoding endogenous gene and the gene-silencing effect obtained by introduction of the silencing RNA is increased when compared to a corresponding plant or cell wherein the Dicer-like 3 protein level is not modified. Alternatively, the modulation of the functional level of the Dicer-like 3 may be an increase in the functional level, achieved e.g. by introduction into the plant cell of a chimeric gene comprising operably linked DNA regions such as a plant-expressible promoter, a DNA region encoding a DCL3 protein and a transcription termination and polyadenylation region functional in plant cells, and the gene-silencing effect obtained by introduction of the silencing RNA is decreased when compared to a corresponding plant or cell wherein the Dicer-like 3 protein level is not modified. The silencing RNA may be a dsRNA molecule which is introduced in the plant cell by transcription in the cell of a chimeric gene comprising a plant-expressible promoter, a DNA region which when transcribed yields an RNA molecule, the RNA molecule comprising a sense and antisense nucleotide sequence, the sense nucleotide sequence comprising about 19 contiguous nucleotides having at least about 90% to about 100% sequence identity to a nucleotide sequence of about 19 contiguous nucleotide sequences from the RNA transcribed from a gene of interest comprised within the plant cell; the antisense nucleotide sequence comprising about 19 contiguous nucleotides having at least about 90 to 100% sequence identity to the complement of a nucleotide sequence of about 19 contiguous nucleotide sequence of the sense sequence; wherein the sense and antisense nucleotide sequence are capable of forming a double stranded RNA by basepairing with each other. Preferably, the sense and antisense nucleotide sequences basepair along their full length, i.e. they are fully complementary.

In yet another embodiment, the invention provides a method for reducing the expression of a gene of interest in a eukaryotic cell, the method comprising the step of providing a silencing RNA molecule to the cell, wherein said cell comprises a functional level of Dicer protein, preferably DCL3 or DCL4, which is different from the level thereof in a corresponding wild-type cell. The silencing RNA molecule may be any silencing RNA molecule as described herein.

In yet another embodiment, the invention provides a method for reducing the expression of a gene of interest in a eukaryotic cell, such as a plant cell, the method comprising the step of providing a silencing RNA molecule into the cell, such as the plant cell, wherein processing of the silencing RNA into siRNA comprises a phase involving dsRNA, characterized in that the cell comprises a functional level of Dicer-like 3 protein which is modified, preferably reduced, compared to the functional level of the Dicer-like 3 protein in a corresponding wild-type cell. Preferably, when the functional level of DCL3 protein is reduced in a plant cell, the target gene of interest whose expression is targeted by the silencing RNA molecule, is an endogenous gene or transgene. Preferably, when the functional level of DCL3 protein is increased in the cell, the silencing mechanism involved in virus resistance, particularly against a virus having a double stranded RNA intermediate molecule during its life cycle, can be increased.

The invention also provides a eukaryotic cell, preferably a plant cell comprising a silencing RNA molecule which has been introduced into the cell, wherein processing of the silencing RNA into siRNA comprises a phase involving dsRNA, characterized in that the cell further comprises a functional level of Dicer-like 3 protein which is different from the wild type functional level of Dicer-like 3 protein in a corresponding wild-type cell. The silencing RNA may be transcribed from a chimeric gene encoding the silencing RNA. The functional level of Dicer-like 3 protein may be decreased or increased, preferably increased when the cell is a cell other than a plant cell, and preferably decreased when the cell is a plant cell.

Yet another embodiment of the invention is a chimeric gene comprising the following operably linked DNA molecules:
a. a eukaryotic promoter, preferably a plant-expressible promoter
b. a DNA region encoding a Dicer-like 3 protein, preferably wherein the Dicer-like 3 protein is a protein comprising a double stranded binding domain of type 3, such as a double stranded binding domain comprising an amino acid sequence having at least 50% sequence identity to an amino acid sequence selected from the amino acid sequence of SEQ ID No.: 7 (At_DCL3) from the amino acid at position 1436 to the amino acid at position 1563; the amino acid sequence of SEQ ID No.: 11 (OS_DCL3) from the amino acid at position 1507 to the amino acid at position 1643; the amino acid sequence of SEQ ID No.: 13 (OS_DCL3b) from the amino acid at position 1507 to the amino acid at position 1603; the amino acid sequence of SEQ ID No.: 9 (Pt_DCL3a from the amino acid at position 1561 to the amino acid at position 1669; and
c. a termination transcription and polyadenylation signal which functions in a cell, preferably a plant cell.

The DCL3 protein may have an amino acid sequence having at least 60% sequence identity with the amino acid sequence of SEQ ID Nos.: 7, 9, 11 or 13.

In yet another embodiment, a eukaryotic host cell, such as a plant cell, comprising a chimeric DCL3 encoding gene as herein described is provided.

The invention also relates to the use of a plant or plant cell with modified functional level of a Dicer-like 4 protein to modulate the gene-silencing effect obtained by introduction of silencing RNA involving a double stranded RNA during the processing of the silencing RNA into siRNA, such as a dsRNA or hpRNA. The modulation of the functional level of the Dicer-like 4 may be decreased in the functional level (e.g. achieved by mutation of the Dicer-like 4 protein encoding endogenous gene) whereby the gene-silencing effect obtained by introduction of the silencing RNA will be decreased compared to a corresponding plant or cell wherein the Dicer-like 4 protein level is not modified. Alternatively, the modulation of the functional level of the Dicer-like 4 may be an increase in the functional level, and wherein the gene-silencing effect obtained by introduction of the silencing RNA is increased compared to a plant wherein the Dicer-like 4 protein level is not modified. The increase in the functional level can be conveniently achieved by introduction into the plant cell of a chimeric gene comprising a plant-expressible promoter operably linked to a DNA region encoding a DCL4 protein and a transcription termination and polyadenylation region functional in plant cells. The mentioned silencing RNA may be a dsRNA molecule which is introduced in the plant cell by transcription in the cell of a chimeric gene comprising a plant-expressible promoter; a DNA region which when transcribed yields an RNA molecule, the RNA molecule comprising a sense and antisense nucleotide sequence, the sense nucleotide sequence comprising about 19 contiguous nucleotides having at least about 90 to about 100% sequence identity to a nucleotide sequence of about 19 contiguous nucleotide sequences from the RNA transcribed from a gene of interest comprised within the plant cell; the antisense nucleotide sequence comprising about 19 contiguous nucleotides having at least about 90 to 100% sequence identity to the complement of a nucleotide sequence of about 19 contiguous nucleotide sequence of the sense sequence; wherein the sense and antisense nucleotide sequence are capable of forming a double stranded RNA by basepairing with each other. Preferably, the sense and antisense nucleotide sequences basepair along their full length, i.e. they are fully complementary.

It is also an embodiment of the invention to provide a method for reducing the expression of a gene of interest in a eukaryotic cell, preferably a plant cell, the method comprising the step of introducing a silencing RNA molecule into the cell, wherein processing of the silencing RNA into siRNA comprises a phase involving dsRNA, characterized in that the cell comprises a functional level of Dicer-like 4 protein which is modified compared to the functional level of the Dicer-like 4 protein in a corresponding wild-type cell.

The invention also provides eukaryotic cells, preferably plant cells comprising a silencing RNA molecule which has been introduced into the cell, wherein processing of the silencing RNA into siRNA comprises a phase involving dsRNA, characterized in that the cell further comprises a functional level of Dicer-like 4 protein which is different from the wild type functional level of Dicer like 4 protein in a corresponding wild-type cell. The functional level of Dicer-like 4 protein may be decreased e.g. by mutation of the endogenous gene encoding the Dicer-like 4 protein of a plant cell. The functional level of Dicer-like 4 protein may also be increased e.g. by expression of a chimeric gene encoding a DCL4 protein in a eukaryotic cell.

Yet another embodiment of the invention is a chimeric gene comprising the following operably linked DNA molecules:
a. a eukaryotic promoter, preferably a plant-expressible promoter
b. a DNA region encoding a Dicer-like 4 protein, preferably wherein the Dicer-like 4 protein is a protein comprising a double stranded binding domain of type 4, such as a double stranded binding domain comprises an amino acid sequence having at least 50% sequence identity to an amino acid sequence selected from the amino acid sequence of SEQ ID No.: 1 (At_DCL4) from the amino acid at position 1622 to the amino acid at position 1696; the amino acid sequence of SEQ ID No.: 5 (OS_DCL4) from the amino acid at position 1520 to the amino acid at position 1593; or the amino acid sequence of SEQ ID No.: 3 (Pt_DCL4) from the amino acid at position 1514 to the amino acid at position 1588; and
c. a termination transcription and polyadenylation signal which functions in ti cell, preferably a plant cell.

The DCL4 protein may have an amino acid sequence having at least 60% sequence identity with the amino acid sequence of SEQ ID Nos.: 1, 3 or 15.

In yet another embodiment, a eukaryotic host cell, such as a plant cell, comprising a chimeric DCL4 encoding gene as herein described is provided.

The invention also provides the use of a eukaryotic cell with a modulated functional level of a Dicer protein to reduce the expression of a gene of interest, as well as eukaryotic cells with a modified functional level, particularly increased level, of a Dicer protein, particularly of DCL3 or DCL4.

In yet another embodiment of the invention, a method is provided for modulating, preferably reducing the expression of a target gene in a eukaryotic cell or organism, through the introduction of a silencing RNA encoding chimeric gene into the eukaryotic cell, whereby the eukaryotic cell is modulated in genes that have an influence (e.g. through transcriptional silencing of the silencing RNA encoding chimeric genes) on the initiation or maintenance of gene silencing by the silencing RNA encoding chimeric genes, particularly hairpin RNA encoding chimeric genes. As an example, the eukaryotic cell may be modulated in a gene involved in RNA directed DNA methylation, e.g. methylation at cytosines in CpG, in CpNpG or cytosines in asymmetric context, such as the CMT3 methyltransferase or DRIVE methyltransferases in plants.

### BRIEF DESCRIPTION OF THE FIGURES

FIG. 1. The chromosome locations of DCL genes in Arabidopsis, poplar and rice.

Each chromosome is depicted approximately to scale, within a genome, with its pseudomolecule length in nucleotides provided. The number under each gene is the position on the pseudomolecule of the start of the gene. The regions shown in yellow on poplar chromosomes VIII and X represent the large duplicated and transposed blocks that have been mapped to have been generated between 8 and 13 million years ago (Sterek et al., 2005).

FIG. 2. Locations of domains in DCL and DCR proteins.

Schematic representation of the different domains within Dicer-like and Dicer genes. The linear arrangement of domains typically found in DCL or DCR proteins is depicted above the Figure. DExD: DEAD and DEAH box helicase domain; Helicase_C: Helicase C domain found in helicases and hawse related proteins; Duf283: domain of unknown function with 3 possible zinc ligands found in Dicer protein family; PAZ: Piwi Argonaut Zwille domain; RNAse signature of ribonuclease III proteins; dsRB: double stranded RNA binding motif table contains the locations, in amino acid residues, where the eight different domains can be found in a DCL or DCR molecule. Boxes that have been blacked out represent the absence or failure to detect the presence of the domain in the appropriate DCL or DCR. The genes are named according to the species in which they are found and their DCL, or DCR type. Tt: Tetrahymena thermophila; Cr: Chlamydomonas reinhardtii; Nc: Neurospora crassa; Hs: Homo sapiens; Dm: Drosophila melanogaster; At: Arabidopsis thaliana; Os: Oryza sativa; Pt: Populus trichocarpa. Plant gene IDs are indicated using the nomenclature in which the number preceding the “g” indicates the chromosome and the number after the “g” indicates the nucleotide position of the start of the coding region on the TAIR database, the JGI poplar chromosome pseudomolecules or TIGR build 3 for rice sequences. Spf1: spliceform 1; Spf2: spliceform 2.

FIG. 3. Phylogenetic analysis of rice, poplar and Arabidopsis.

Consensus phylogenetic trees, constructed by neighbour-Joining method with pairwise deletion, using the Dayhof matrix model for amino acid substitution, presented in radial format for [A] the entire DCL molecules and [B] the C-terminal dsRBb domain. The colour coding shows the grouping of DCL types 1, 2, 3 and 4 based on clustering with the Arabidopsis type member. Branches with 100 percent consistence after 1000 bootstrap replications are indicated with black dots.

FIG. 4. Detection of OsDCL2A and OsDCL2B in japonica and indica rice.

PCR analysis of japonica (lane 1) and indica (lane 2) rice using a set of primers that should give a band of 772 nt for the presence of OsDCL2A and a band of 577 nt for the presence of OsDCL2B. The gel indicates that both rice subspecies contain both the 2A and 2B genes.

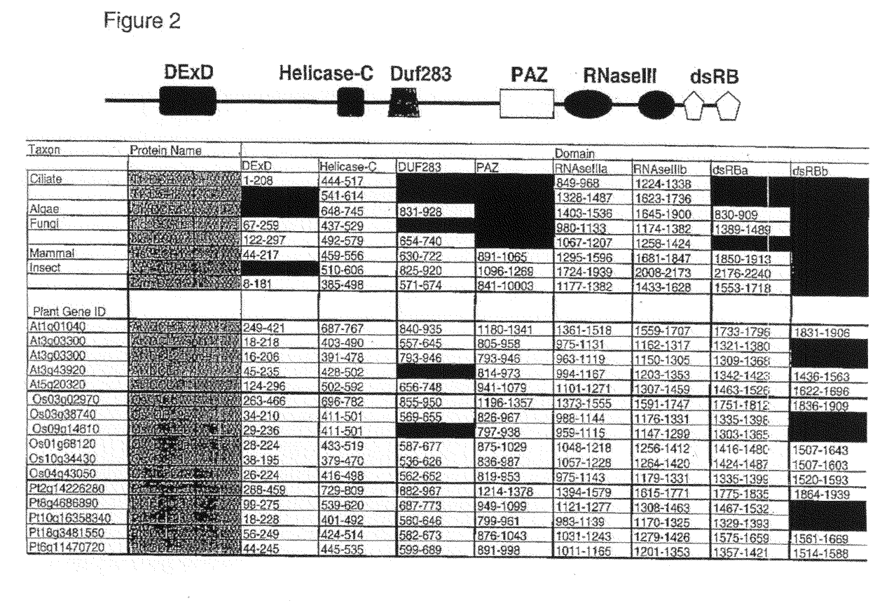

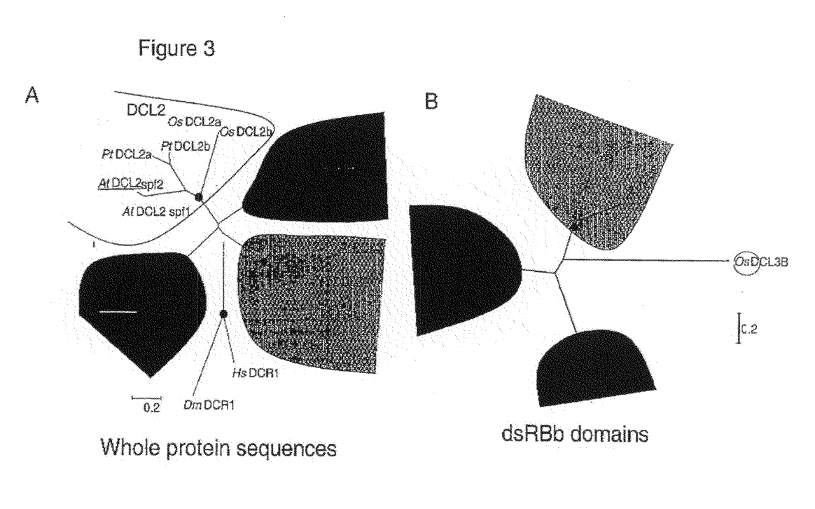

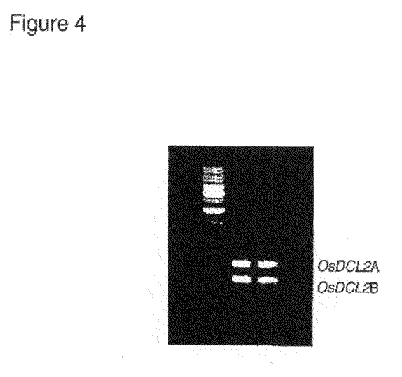

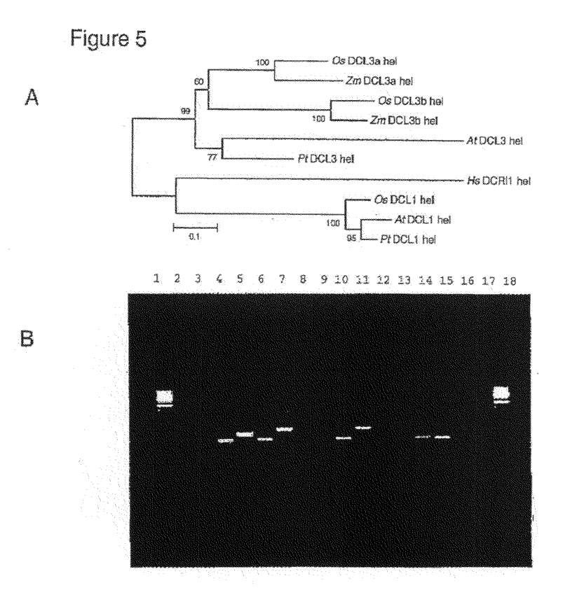

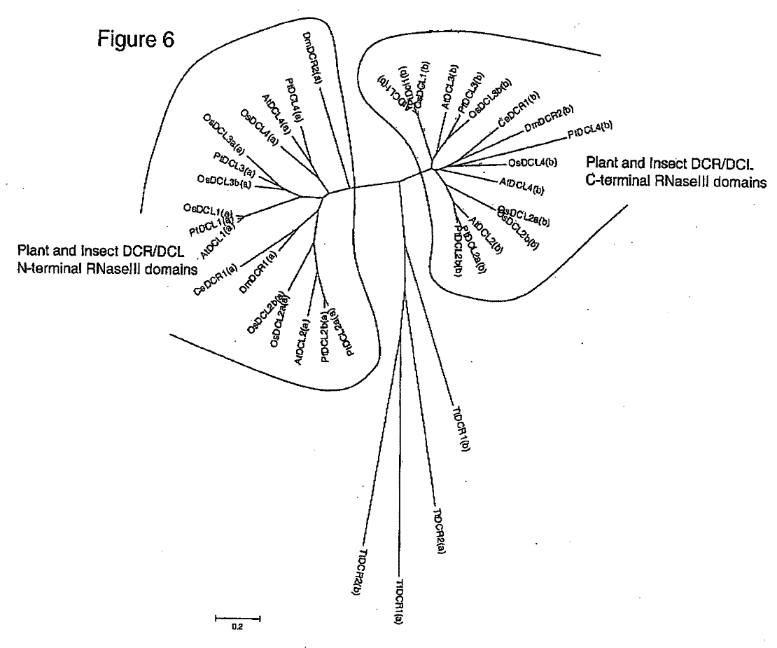

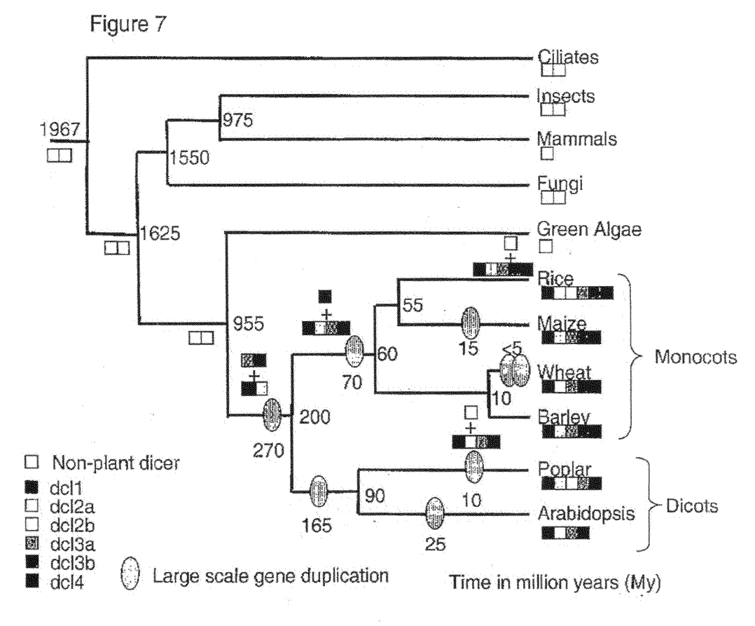

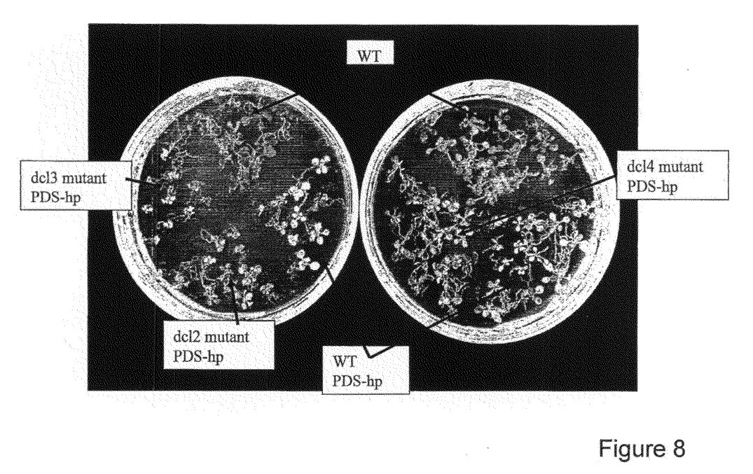

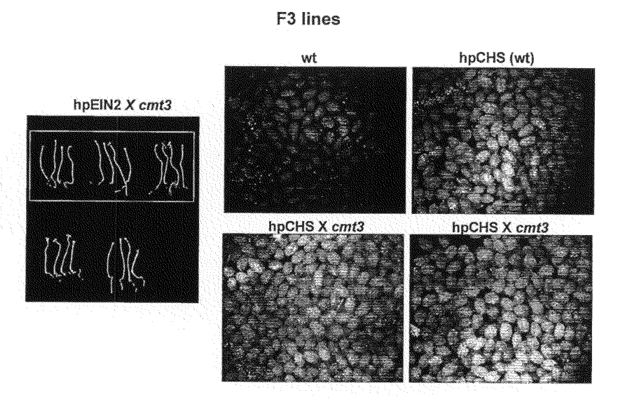

FIG. 5. Detection of DCL3A and DCL3B genes in monocots and their phylogenetic relationships.

[A] The phylogenetic analysis of the helicase-C domains of rice, maize, Arabidopsis and poplar DCL3-type genes, with the inclusion of their DCL1 counterparts to root the tree. The analysis was done in a similar way to that described in FIG. 2. [B] PCR analysis for the detection of DCL3A and DCL3B genes in a range of monocots using A- and B-specific primer pairs. The product from the 3B primers were expected to be larger (˜600 nt) than the product from the detection of DCL3A (˜500 nts). Lanes 1 & 18: markers; lanes 2, 4, 6, 10, 14 and 16 DCL3A-specific primer pairs; lanes 3, 5, 7, 11, 15 and 17 DCL3B-specific primer pairs. Lanes 8 and 12 negative control 3A forward with 3B reverse primers; lanes 9 and 13 negative control 3B forward with 3A reverse primer pairs. Lanes 2 and 3 water control; lanes 4 and 5 rice DNA; lanes 6.9 Triticum DNA; lanes 10-13 barley DNA; lanes 14 and 15 maize DNA and lanes 16 and 17 Arabidopsis DNA. The results show the detection of DCL3A and DCL3B in all of the monocots DNA tested.

FIG. 6. Phylogenetic analysis of RNAse III domains of plants, insects and ciliates. The analysis was done essentially as described in FIG. 2. The coloured regions show that the N-terminal RNaseIII domains from rice, Arabidopsis, poplar, C. elegans, Drosophila, and Tetrahymena all form one cluster while the C-terminal RNaseIII domains show a similar counterpart cluster.

FIG. 7. Proposed evolutionary tree of Dicer genes in plants.

The presence or absence of different DCL genes and the times of divergence of the different nodes are depicted on the currently accepted phylogenetic tree of species. Branch lengths are not to scale. The estimated large scale gene duplication events are depicted by blue ellipses. The numbers at the nodes and at the ellipses are estimated dates in million years (my). These numbers are rounded to the nearest 5 my, and for dates that have been previously estimated in ranges, the median of that range has been taken. The different plant DCL types are colour coded and the non-plant dicer genes are represented as white boxes. The duplication of a DCL gene is indicated by as addition (+) sign. The phylogenetic tree with its times of divergence and large scale duplication events are based on the calculations and phylogenetic trees of Blane & Wolfe (2004) [20]. Hedges et al., (2004) [27] and Sterek et al., (2005) [19].

FIG. 8: Phenotypes of silencing achieved by a chimeric gene encoding a double stranded RNA molecule comprising complementary sense and antisense RNA targeted towards phytoene desaturase (PDS-hp) in Arabidopsis seedlings of different genetic backgrounds. WT: wild type A. thaliana (without PDS-hp); WT PDS-hp: Wild type A. thaliana with PDS-hp gene. dcl2: mutant A. thaliana wherein Dicer like 2 gene is inactivated. Dcl3: mutant A. thaliana wherein Dicer like 3 gene is inactivated. Dcl4: mutant A. thaliana wherein Dicer like 4 gene is inactivated. The degree of bleaching is a measure of the degree of silencing.

FIG. 9: The effect of CMT3 mutation on hpRNA-mediated EIN2 and CHS silencing.

Left panel: The length of hypocotyls grown in the dark on ACC containing medium, is generally longer for the F3 hpEIN2 plants with the homozygous cmt3 mutation than with the wild-type background (wt), indicating stronger EIN2 silencing in the cmt3 background. The transgenic plants inside the box have the mutant background, while the transgenic plants outside the box have the wild-type background.

Right panel: the seed coat color is significantly lighter for the hpCHS plants with the cmt3 background than with the wild-type background, indicative of stronger CHS silencing in the former transgenic plants.

Table 1. Variation within and between DCLs of rice, poplar and Arabidopsis.

The variations are Oven as number of amino acid changes (to the nearest integer), and were calculated using MEGA 3.1 using the complete deletion option and assuming uniform rates among sites. The number in brackets indicates the standard error (to the nearest integer). The variability between DCLs is net variability.

Table 2. Pairwise distances between DCLS of rice, poplar and Arabidopsis.

### DETAILED DESCRIPTION OF THE INVENTION

The current invention is based on the demonstration by the inventors that modulating the functional level of several types of Dicer-like proteins in eukaryotic cells, such as plants modulates the gene-silencing effect achieved by the introduction of double stranded RNA molecules, particularly hairpin RNA into such cells. In another aspect, the invention is based on the demonstration by the inventors that, the gene-silencing effect achieved by silencing RNA-encoding chimeric genes, particularly hairpin RNA encoding chimeric genes, can be modulated by modulating genes in eukaryotic cells which influence the initiation or maintenance of gene silencing.

In particular, it was demonstrated that gene-silencing achieved by chimeric genes encoding a double stranded RNA molecule (particularly a hpRNA) in plant cells lacking functional DCL3 protein was unexpectedly enhanced. Further it was also found that gene-silencing achieved by chimeric genes encoding a double stranded RNA molecule, particularly a hpRNA molecule, in plant cells lacking functional DCL4 protein was reduced leading to the realization that increase in the functional level of DCL4 protein could lead to a stronger gene-silencing effect achieved by introduction of double-stranded RNA molecules into such plant cells. In addition, it was demonstrated that gene-silencing achieved by chimeric genes encoding a double stranded RNA molecule (particularly a hpRNA) in plant cells lacking functional CMT3 methyltransferase protein was unexpectedly enhanced.

Accordingly, the invention provides a method for modulating the gene-silencing effect in a eukaryotic cell or organism achieved by introduction of a gene silencing molecule, such as a gene-silencing RNA preferably encoded by a gene-silencing chimeric gene, by modulation or alteration of the functional level of a Dicer protein, including a DCL protein, such as DCL3 or DCL4, which Dicer protein or DCL protein is involved, directly or indirectly, in processing of artificially introduced dsRNA molecules, particularly of hpRNA molecules, particularly long hpRNA molecules into short-interfering siRNA of 21-24 nt.

As used herein, “artificially introduced dsRNA molecule” refers to the direct introduction of dsRNA molecule, which may e.g. occur exogenously, i.e. after synthesis of the dsRNA outside of the cell, or endogenously by transcription from a chimeric gene encoding such dsRNA molecule, however it does not refer to the conversion of a single stranded RNA molecule into a dsRNA inside the eukaryotic cell or plant cell.

As used herein, a “Dicer protein” is a protein having ribonuclease activity which is involved in the processing of double stranded RNA molecules into short interfering RNA (siRNA). The ribonuclease activity is so-called ribonuclease III activity, which predominantly or preferentially cleaves double stranded RNA substrates rather than single-stranded RNA molecules, thereby targeting the double stranded portion of a RNA molecule. Typically, the double stranded RNA substrate comprises a double stranded region having at least 19 contiguous basepairs. Alternatively, the double stranded RNA substrate may be a transcript which is processed to form a miRNA. The term Dicer includes Dicer-like (DCL) proteins which are proteins that show a high degree of similarity to Dicers and which are presumed to be functional based on their expression in a cell. Such relationships may readily be identified by those skilled in the art. Dicer proteins are preferentially involved in processing the double-stranded RNA substrates into siRNA molecules of about 21 to 24 nucleotides in length.

As used herein “gene-silencing effect” refers to the reduction of expression of a target nucleic acid in a host cell, preferably a plant cell, which can be achieved by introduction of a silencing RNA. Such reduction may be the result of reduction of transcription, including via methylation and/or chromatin remodeling, or post-transcriptional modification of the RNA molecules, including via RNA degradation, or both. Gene-silencing should not necessarily be interpreted as an abolishing of the expression of the target nucleic acid or gene. It is sufficient that the level expression of the target nucleic acid in the presence of the silencing RNA is lower that in the absence thereof. The level of expression may be reduced by at least about 10% or at least about 15% or at least about 20% or at least about 25% or at least about 30% or at least about 35% or at least about 40% or at least about 45% or at least about 50% or at least about 55% or at least about 60% or at least about 65% or at least about 70% or at least about 75% or at least about 80% or at least about 85% or at least about 90% or at least about 95% or at least about 100%. Target nucleic acids may include endogenous genes, transgenes or viral genes or genes introduced by viral vectors. Target nucleic acid may further include genes which are stably introduced in the host's cell genome, preferably the host cell's nuclear genome. Preferably, gene silencing is a sequence-specific effect, wherein expression of the target nucleic acid is specifically reduced compared to other nucleic acids in the cell, although the target nucleic acid may represent a family of related sequences.

As used herein, “silencing RNA” or silencing RNA molecule refers to any RNA molecule which upon introduction into a host cell, preferably a plant cell, reduces the expression of a target gene. Such silencing RNA may e.g. be so-called “antisense RNA”, whereby the RNA molecule comprises a sequence of at least 20 consecutive nucleotides having at least 95% sequence identity to the complement of the sequence of the target nucleic acid, preferably the coding sequence of the target gene. However, antisense RNA may also be directed to, regulatory sequences of target genes, including the promoter sequences and transcription termination and polyadenylation signals. Silencing RNA further includes so-called “sense RNA” whereby the RNA molecule comprises a sequence of at least 20 consecutive nucleotides having at least 95% sequence identity to the sequence of the target nucleic acid. Without intending to limit the invention to any particular mode of action, it is generally believed that single stranded silencing RNA such as antisense RNA or sense RNA is converted into a double stranded intermediate e.g. through the action of RNA dependent RNA polymerase, whereby the double stranded intermediate is processed to form 21-24 nt short interfering RNA molecules.

The mentioned sense or antisense RNA may of course be longer and be about 50 nt, about 100 nt, about 200 nt, about 300 nt, about 500 nt, about 1000 nt, about 2000 nt or even about 5000 nt or larger in length, each having an overall sequence identity of respectively about 40%, 50%, 60%, 70%, 80%, 90% or 100% with the nucleotide sequence of the target nucleic acid (or its complement) The longer the sequence, the less stringent the requirement for the overall sequence identity. However, the longer sense or antisense RNA molecules with less overall sequence identity should at least comprise 20 consecutive nucleotides having at least 95% sequence identity to the sequence of the target nucleic acid or its complement.

Other silencing RNA may be “unpolyadenylated RNA” comprising at least 20 consecutive nucleotides having at least 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 or WO2005/026356 (both documents herein incorporated by reference) comprising at least 20 consecutive nucleotides having at least 95% sequence identity to the sequence of the target nucleic acid or the complement thereof, and further comprising a largely-double stranded region us described in WO03/07.6619 or WO2005/026356 (including largely double stranded regions comprising a nuclear localization signal from a viroid of the Potato spindle tuber viroid-type or comprising CUG trinucleotide repeats). Silencing RNA may also be double stranded RNA comprising a sense and antisense strand as herein defined, wherein the sense and antisense strand are capable of base-pairing with each other to form a double stranded RNA region (preferably the said at least 20 consecutive nucleotides of the sense and antisense RNA are complementary to each other. The sense and antisense region may also be present within one RNA molecule such that a hairpin RNA (hpRNA) can be formed when the sense and antisense region form a double stranded RNA region. hpRNA is well-known within the art (see e.g WO99/53050, herein incorporated by reference). The hpRNA may be classified as long hpRNA, having long, sense and antisense regions which can be largely complementary, but need not be entirely complementary (typically larger than about 200 bp, ranging between 200-1000 bp). hpRNA can also be rather small ranging in size from about 30 to about 42 bp, but not much longer than 94 hp (sec WO04/073390, herein incorporated by reference). Silencing RNA molecules could also comprise so-called microRNA or synthetic or artificial microRNA molecules or their precursors, as described e.g. in Schwab et al. 2006, Plant Cell 18(5):1121-1133.

Silencing RNA can be introduced directly into the host cell after synthesis outside of the cell, or indirectly through transcription of a “gene-silencing chimeric gene” introduced into the host cell such that expression of the chimeric gene from a promoter in the cell gives rise to the silencing RNA. The gene-silencing chimeric gene may be introduced stably into the host cells (such us a plant cell) genuine, preferably nuclear genome, or it may be introduced transiently. The silencing RNA molecules are preferably introduced into the host cell, or heterologous silencing RNA molecules, or silencing RNA molecules non-naturally occurring in the eukaryotic host cell, or artificial silencing RNA molecules.

As used herein, “modulation of functional level” means either an increase or decrease in the functional level of the concerned protein. “Functional level” should be understood to refer to the level of active protein, in casu the level of protein capable of performing the ribonuclease III activity associated with Dicer or DCL. The functional level is a combination of the actual level of protein present in the host cell and the specific activity of the protein. Accordingly, the functional level may e.g. be modified by increasing or decreasing the actual protein concentration in the host cell. The functional level may also be modulating the specific activity of the protein. Such increase or decrease of the specific activity may be achieved by expressing a variant protein, such as a non-naturally occurring or man-made variant with higher or lower specific activity (or by replacing the endogenous gene encoding the relevant DCL protein with an allele encoding such a variant). Increase or decrease of the specific activity may also be achieved by expression of an effector molecule, such as e.g. an antibody directed towards such a DCL protein and which affects the binding of dsRNA molecules or the catalytic RNAse III activity.

Increase of DCL3 activity in a plant cell will lead to a reduced gene silencing effect achieved by silencing RNA, the processing of which involves a dsRNA molecule, including sense RNA, antisense RNA, unpolyadenylated sense and antisense RNA, sense or antisense RNA having, a largely doubled stranded RNA region, and double stranded RNA comprising a sense and antisense regions which are capable of forming a ds stranded RNA region, particularly silencing RNA targeted to reduce the expression of endogenous genes, or trangenes. In the case of virus resistance, particularly where the virus has a double-stranded RNA phase, the gene silencing effect may be enhanced. Decrease of the DCL 3 activity will yield to an enhanced silencing effect achieved by silencing RNA, particularly silencing RNA targeted towards endogenes or transgenes, but may result in reduced gene silencing for viral nucleic acids. Inversely, increase of DCL4 activity in a plant cell will leaded to increase the gene silencing effect achieved by the silencing RNA, while decrease of DCL4 activity will yield a reduced gene silencing effect.

Increase of DCL activity can be conveniently achieved by overexpression, i.e. through the introduction of a chimeric gene into the host cell or plant cell comprising a region DNA region coding for an appropriate DCL protein operably linked to a promoter region and transcription termination and polyadenylation signals functional in host cell or the plant cell. Increase can however also be achieved by mutagenesis and selection-identification of the individual host/plant cell, host/plant cell line or host/plant having a higher activity of the DCL protein than the starting material.

A decrease in DCL activity can be conveniently achieved by mutagenesis and selection-identification of the individual host/plant cell, host/plant cell line or host/plant having a lower activity of the DCL protein than the starting material. A decrease in DCL activity can also be achieved by gene-silencing whereby the targeted gene whose expression is to be reduced is the gene encoding the DCL protein. In case of reduction of DCL3 gene expression through gene silencing the silencing RNA could be any silencing RNA which is processed into a dsRNA form during siRNA genesis. Downregulation of DCL4 gene expression however will require use of an alternative gene-silencing pathway such as use of artificial micro-RNA molecules as described e.g. in WO2005/052170, WO2005/047505 or US 2005/0144667 (all documents incorporated herein by reference)

As indicated above, “Dicer or Dicerlike proteins involved in processing of artificially introduced dsRNA molecules” include DCL 3 and DCL4 proteins. As used herein a “plant dicer” or plant “dicer-like” protein is a protein having ribonuclease activity on double stranded RNA substrates (ribonuclease III activity) which is characterized by the presence of at least the following domains: a DExD or DExH domain (DEAD/DEAH domain), a Helicase-C domain, preferably a Duf283 domain which may be absent, a PAZ domain, two RNAse III domains and at least one and preferably 2 dsRB domains.

Helicase C: The domain, which defines this group of proteins is found in a wide variety of helicases and helicase related proteins. It may be that this is not an autonomously folding unit, but an integral part of the helicase (PF00271; IPR001650)

PAZ domain: This domain is named after the proteins Piwi Argonaut and Zwille. It is also found in the CAP protein from Arabidopsis thaliana. The function of the domain is unknown but has been found in the middle region of a number of members of the Argonaute protein family, which also contain the Piwi domain in their C-terminal region. Several members of this family have been implicated in the development and maintenance of stem cells through the RNA-mediated gene-quelling mechanisms associated with the protein Dicer. (PF02170; IPR003100)

Duf283: This putative domain is found in members of the Dicer protein family. This protein is a dsRNA nuclease that is involved in RNAi and related processes. This domain of about 100 amino acids has no known function, but does contain 3 possible zinc ligands. (PF03368, IPR005034).

DExD: Members of this family include the DEAD and DEAH box helicases. Helicases are involved in unwinding nucleic acids. The DEAD box helicases are involved in various aspects of RNA metabolism, including nuclear transcription, pre mRNA splicing, ribosome biogenesis, nucleocytoplasmic transport, translation, RNA decay and organellar gene expression (PF00270, IPR011545).

RNAse III: signature of the ribonuclease III proteins (PF00636, IPR000999)

DsRB (Double stranded RNA binding motif): Sequences gathered for seed by HMM_iterative_training Putative motif shared by proteins that bind to dsRNA. At least some DSRM proteins seem to bind to specific RNA targets. Exemplified by Staufen, which is involved in localisation of at least live different mRNAs in the early Drosophila embryo. Also by interferon-induced protein kinase in humans, which is part of the cellular response to dsRNA (PF00035, IPR001159).

These domains can easily be recognized by computer based searches using e.g. PROSITE profiles PDOC50821 (PAZ domain), PDOC00448 (RNase III domain), PDOC50137 (dsRB domain) and PDOC00039 (DExD/DexH domain) (PROSITE is available at www.expasy.ch/prosite). Alternatively, the BLOCKS database and algorithm (blocks.fhcrc.org) may be used to identify PAZ(IPB003100) or DUF283(IPB005034) domains. Other databases and algorithms are also available (pFAM: http://www.sanger.ae.uk/Software/Pfam/ INTERPRO: http://www.cbi.ae.uk/interpro/; the above cited PF numbers refer to pFAM database and algorithm and IPR numbers to the INTERPRO database and algorithm).

Typically, a DCL2 protein will process double stranded RNA into short interfering RNA molecules of about 22 nucleotides, a DCL3 protein will process double stranded RNA into short interfering RNA molecules of about 24 nucleotides, and DCL4 will process double stranded RNA into short interfering RNA molecules of about 21 nucleotides.

As used herein a “Dicer-like 3 protein (DCL3)” is a plant dicer-like protein further characterized in that it has two dsRB domains (dsRBa and dsRBb) wherein the dsRBb domain is of type 3. Preferably, dsRBb has an amino acid sequence having at least 50% sequence identity to an amino acid sequence selected from the following sequences:
the amino acid sequence of SEQ ID No.: 7 (At_DCL3) from the amino acid at position 1436 to the amino acid at position 1563;
the amino acid sequence of SEQ ID No.: 11 (OS_DCL3) from the amino acid at position 1507 to the amino acid at position 1643;
the amino acid sequence of SEQ ID No.: 13 (OS_DCL3b) from the amino acid at position 1507 to the amino acid at position 1603;
the amino acid sequence of SEQ ID No.: 9 (Pt_DCL3a) from the amino acid at position 1561 to the amino acid at position 1669.

The dsRBb domain may of course have a higher sequence identity to the cited dsRBb domains such as at least 55%, at least 60%, at least 65%, at least 70%, at least 75%, at least 80%, at least 85%, at least 90%, at least 95% or be identical with the cited amino acid sequences.

Nucleotide sequences encoding Dicer-like 3 enzymes can also be identified as those nucleotide sequences encoding a Dicer-like enzyme and which upon PCR amplification with a set of DCL3 diagnostic primers such as primers having the nucleotide sequence of SEQ II) No.: 31 and SEQ ID No.: 32 yields a DNA molecule of about 600 nt in length or upon PCR amplification with a set of DCL3 diagnostic primers such as primers having the nucleotide sequence of SEQ ID No.: 35 and SEQ ID No.: 36 yields a DNA molecule or upon PCR amplification with a set of DCL3 diagnostic primers such as primers having the nucleotide sequence of SEQ ID No.: 37 and SEQ ID No.: 38 yields a DNA molecule.

Fragments of nucleotide sequences encoding Dicer-like 3 enzymes can further be amplified using primers comprising the nucleotide sequence of SEQ ID No.: 15 and SEQ ID No.: 16 or the nucleotide sequence of SEQ ID No.: 17 and SEQ ID No.: 18 or the nucleotide sequence of SEQ ID No.: 19 and SEQ ID No.: 20 or the nucleotide sequence of SEQ ID No.: 21 and SEQ ID No.: 22. The obtained fragments can be joined to each other using standard techniques. Accordingly, suitable DCL3 encoding nucleotide sequences may include a DNA nucleotide sequence amplifiable with the primers of SEQ ID No.: 15 and SEQ ID No.: 16 or with primers of SEQ ID No.: 17 and SEQ ID No.: 18 or with primers of SEQ ID No.:19 and SEQ ID No.: 20 or with primers of SEQ ID No.:21 and SEQ ID No.: 22.

Further suitable nucleotide sequences encoding Dicer-like 3 enzymes are those which encode a protein comprising an amino acid sequence of at least about 60% or at least about 65% or at least about 70% or at least about 75% or at least about 80% or at least about 85% or at least about 90% or at least about 95% sequence identity or being essentially identical with the proteins comprising an amino acid sequence of SEQ ID Nos.: 7 or 9 or 11 or 13 or with the proteins having amino acid sequences available from databases with the following accession numbers: NP_{—}189978.

Such nucleotide sequences include the nucleotide sequences of SEQ ID Nos.: 8 or 10 or 12 or 14 or nucleotide sequences with accession numbers: NM_{—}114260 or nucleotide sequences encoding a dicer-like 3 protein, wherein the nucleotide sequences have at least about 60% or at least about 65% or at least about 70% or at least about 75% or at least about 80% or at least about 85% or at least about 90% or at least about 95% sequence identity to these sequences or being essentially identical thereto.

As used herein a “Dicer-like 4 protein (DCL4)” is a plant dicer-like protein further characterized in that it has two dsRB domains (dsRBa and dsRBb) wherein the dsRBb domain is of type 4. Preferably, dsRBb has an amino acid sequence having at least 50% sequence identity to an amino acid sequence selected from the following sequences:
the amino acid sequence of SEQ ID No.: 1 (At_DCL4) from the amino acid at position 1622 to the amino acid at position 1696;
the amino acid sequence of SEQ No.: 5 (OS_DCL4) from the amino acid at position 1520 to the amino acid at position 1593; or
the amino acid sequence of SEQ ID No.: 3 (Pt_DCL4) from the amino acid at position 1514 to the amino acid at position 1588.

The dsRBb domain may of course have a higher sequence identity to the cited dsRBb domains such as at least 55%, at least 60%, at least 65%, at least 70%, at least 75%, at least 80%, at least 85%, at least 90%, at least 95% or be identical with the cited amino acid sequences.

Nucleotide sequences encoding Dicer-like 4 enzymes can also be identified as those nucleotide sequences encoding a Dicer-like enzyme and which upon PCR amplification with a set of DCL4 diagnostic primers such as primers having the nucleotide sequence of SEQ ID No.: 33 and SEQ ID No.: 34 yields a DNA molecule, preferably of about 920 bp or about 924 bp in length.

Fragments of nucleotide sequences encoding Dicer-like 4 enzymes can further be amplified using primers comprising the nucleotide sequence of SEQ ID No.: 23 and SEQ ID No.: 24 or the nucleotide sequence of SEQ ID No.: 25 and SEQ ID No.; 26 or the nucleotide sequence of SEQ ID No.: 27 and SEQ ID No.: 28 or the nucleotide sequence of SEQ ID No.: 29 and SEQ ID No.: 30. The obtained fragments can be joined to each other using standard techniques. Accordingly, suitable DCL4 encoding nucleotide sequences may include a DNA nucleotide sequence amplifiable with the primers of SEQ ID No.: 23 and SEQ ID No.: 24 or with primers of SEQ ID No.: 25 and SEQ ID No.: 26 or with primers of SEQ ID No.:27 and SEQ ID No.: 28 or with primers of SEQ ID No.: 29 and SEQ ID No.: 30.

Further suitable nucleotide sequences encoding Dicer-like 4 proteins are those which encode a protein comprising an amino acid sequence of at least about 60% or 65% or 70% or 75% or 80% or 85% or 90% or 95% sequence identity or being essentially identical with the proteins comprising an amino acid sequence of SEQ ID Nos.: 1 or 3 or 5 or with the proteins having amino acid sequences available from databases with the following accession numbers: AAZ80387; P84634.

Such nucleotide sequences include the nucleotide sequences of SEQ ID Nos.: 2 or 4 or 6 or nucleotide sequences with accession numbers: NM_{—}122039; DQ118423 or nucleotide sequences encoding a dicer-like 4 protein, wherein the nucleotide sequences have at least about 60% or at least about 65% or at least about 70% or at least about 75% or at least about 80% or at least about 85% or at feast about 90% or at least about 95% sequence identity to these sequences or being essentially identical thereto.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch 1970) The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3. Sequences are indicated as “essentially similar” when such sequence have a sequence identity of at least about 75%, particularly at least about 80%, more particularly at least about 85%, quite particularly about 90%, especially about 95%, more especially about 100%, quite especially are identical. It is clear than when RNA sequences are the to be essentially similar or have a certain degree of sequence identity with DNA sequences, thymine (T) in the DNA sequence is considered equal to uracil (U) in the RNA sequence. Thus when it is stated in this application that a sequence of 19 consecutive nucleotides has at least 94% sequence identity to a sequence of 19 nucleotides, this means that at least 18 of the 19 nucleotides of the first sequence are identical to 18 of the 19 nucleotides of the second sequence.

In one embodiment of the invention, a method for reducing the expression of a nucleic acid of interest in a host cell, preferably a plant cell is provided, the method comprising the step of introducing a dsRNA molecule into a host cell, preferably plant cell, said dsRNA molecule comprising a sense and antisense nucleotide sequence, whereby the sense nucleotide sequence comprises about 19 contiguous nucleotides having at least about 90 to about 100% sequence identity to a nucleotide sequence of about 19 contiguous nucleotide sequences from the RNA transcribed (or replicated) from the nucleic acid of interest and the antisense nucleotide sequence comprising about 19 contiguous nucleotides having at least about 90%, such as about 94% to 100% sequence identity to the complement of a nucleotide sequence of about 19 contiguous nucleotide sequence of the sense sequence and wherein said sense and antisense nucleotide sequence are capable of forming a double stranded RNA by basepairing with each other, characterized in that the host cell, preferably a plant cell comprises a functional level of Dicer-like 4 protein which is modified compared to the functional level of said Dicer-like 4 protein in a wild-type host cell, preferably a plant cell. The functional level Dicerlike 4 protein can be increased conveniently by introduction of a chimeric gene comprising a promoter region and a transcription termination and polyadenylation signal operably linked to a DNA region coding for a DCL4 protein, the latter being as defined elsewhere in this application.

As used herein, the term “promoter” denotes any DNA which is recognized and bound (directly or indirectly) by a DNA-dependent RNA-polymerase during initiation of transcription. A promoter includes the transcription initiation site, and binding sites for transcription initiation factors and RNA polymerase, and can comprise various other sites (e.g., enhancers), at which gene expression regulatory proteins may bind.

The term “regulatory region”, as used herein, means any DNA, that is involved in driving transcription and controlling (i.e., regulating) the timing and level of transcription of a given DNA sequence, such as a DNA coding for a protein or polypeptide. For example, a 5′ regulatory region (or “promoter region”) is a DNA sequence located upstream (i.e., 5′) of a coding sequence and which comprises the promoter and the 5′-untranslated leader sequence. A 3′ regulatory region is a DNA sequence located downstream (i.e., 3′) of the coding sequence and which comprises suitable transcription termination (and/or regulation) signals, which may include one or, more polyadenylation signals.

In one embodiment of the invention the promoter is a constitutive promoter. In another embodiment of the invention, the promoter activity is enhanced by external or internal stimuli (inducible, promoter), such as but not limited to hormones, chemical compounds, mechanical impulses, abiotic or biotic stress conditions. The activity of the promoter may also be regulated in a temporal or spatial manner (tissue-specific promoters; developmentally regulated promoters). The promoter may be a viral promoter or derived front a viral genome.

In a particular embodiment of the invention, the promoter is a plant expressible promoter. As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Hapster et al., 1988), the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al., 1996), stem-specific promoters (Keller et al., 1988), leaf specific promoters (Hudspeth et al., 1989), mesophyl-specific promoters (such as the light-inducible Rubisco promoters), root-specific promoters (Keller et al., 1989), tuber-specific promoters (Keil et al., 1989), vascular tissue specific promoters (Peleman et al., 1989), stamen-selective promoters (WO 89/10396, WO 92/13956), dehiscence zone specific promoters (WO 97/13865) and the like.

In another embodiment of the invention, a method for reducing the expression of a nucleic acid of interest in a host cell, preferably a plant cell is provided, the method comprising the step of introducing a dsRNA molecule into a host cell, preferably plant cell, said dsRNA molecule comprising a sense and antisense nucleotide sequence, whereby the sense nucleotide sequence comprises about 19 contiguous nucleotides having at least about 90%, such as at least 94%, to about 100% sequence identity to a nucleotide sequence of about 19 contiguous nucleotide sequences from the RNA transcribed (or replicated) from the nucleic acid of interest and the antisense nucleotide sequence comprising about 19 contiguous nucleotides having at least about 90%, such as about 94% to about 100% sequence identity to the complement of a nucleotide sequence of about 19 contiguous nucleotide sequence of the sense sequence and wherein said sense and antisense nucleotide sequence are capable of forming a double stranded RNA by basepairing with each other, characterized in that the host cell, preferably a plant cell comprises a functional level of Dicer-like 4 protein which is reduced compared to the functional level of said Dicer-like 4 protein in a corresponding wild-type host cell, preferably a plant cell. Such a reduction could be achieved by mutagenesis of host cells or plant cells, host cell lines or plant cell lines, hosts or plants or seeds, followed by identification of those host cells or plant cells, host cell lines or plant cell lines, hosts or plants or seeds wherein the Dicer-like 4 activity has been reduced or abolished. Mutants having a deletion or other lesion in the DCL 4 encoding glue can conveniently be recognized using e.g. a method named “Targeting induced local lesions IN genomes (TILLING)”. Plant Physiol. 2000 June; 123(2):439-42.

Preferably, the sense and antisense nucleotide sequences of dsRNA molecules as described herein basepair along their full length, i.e. they are fully complementary. “Basepairing” as used herein includes G:U basepairs as well as A:U and G:C basepairs. Alternatively, the dsRNA molecules may be a transcript which is processed to form a miRNA. Such molecules typically fold to form double stranded regions in which 70-95% of the nucleotides are basepaired, e.g. in a region of 20 contiguous nucleotides, 1-6 nucleotides may be non-basepaired.

In yet another embodiment of the invention, the use of a plant or plant cell with a modified functional level of DCL3 protein is provided to modulate the gene silencing effect obtained by introduction of silencing RNA requiring a double stranded RNA phase during processing into siRNA such as e.g. dsRNA or hpRNA or genes encoding such silencing RNA. A preferred embodiment of the invention is the use of a plant or plant cell with a reduced level of DCL3 protein, particularly a plant or plant cell which does not contain functional DCL3 protein. Gene silencing using silencing RNA requiring a double stranded RNA phase during the processing into siRNA is enhanced in such a genetic background.

In yet another embodiment of the invention, the use of a plant or plant cell with a modified functional level of DCL3 protein is provided to modulate virus resistance of such a plant cell. A preferred embodiment of the invention is the use of a plant or plant cell with an increased level of DCL3 protein.

Although not intending to limit the invention to a particular mode of action, it may be that the enhanced gene-silencing effect for endogene or transgene silencing is due to reduced transcriptional silencing of the silencing RNA, particularly hpRNA, encoding transgenes in this genetic background. Silencing should also be enhanced in other silencing-deficient mutants where transcriptional silencing is relieved such as in pol iv and rdr2 background.

However, DCL3 may also cleave hpRNA stems compromising RNAi by removing substrate that would otherwise be processed by DCL2 and DCL4 into 21 and 22 nt siRNA molecules. It has been demonstrated that silencing of the target gene by silencing RNA, particularly hpRNA, encoding transgenes by is enhanced in silencing deficient mutants where transcriptional silencing is relieved including rdr2 and cmt3 background.

A dcl3 genetic background in a plant cell, which is suitable for the methods according to the invention can be conveniently achieved by insertion mutagenesis (e.g. using a T-DNA or transposon insertion mutagenesis pathway, whereby insertions in the region of the endogenous DCL3 encoding gene are identified, according to methods well known in the art. Similar genetic dcl3 genetic background can be achieved using chemical mutagenesis whereby plants with a reduced level of DCL3 are identified. Plants with a lesion in the genome region of a DCL3 encoding gene can be conveniently identified using the so-called TILLING methodology (supra).

DCL3 alleles can also be exchanged for less or non-functional DCL3 encoding alleles through homologous recombination methods using targeted double stranded break induction (e.g. with rare cleaving double stranded break inducing enzymes such as homing endonucleases).

Preferred, less functional, mutant alleles are those having an insertion, substitution or deletion in a conserved domain such as the DExD, Helicase-C, Duf 283, PAZ, RnaseIII and dsRB domains whose location in the different identified DCL3 proteins is indicated in FIG. 2.

The methods according to the invention can be used in various ways. One possible application is the restoration of weak silencing loci obtained by introduction of chimeric genes yielding silencing RNA, preferably hpRNA, into cells of a plant, by introduction of such weak silencing loci into a dcl3 genetic background (with reduced functional level of DCL3) or into a DCL4 overexpressing background. Another utility of the methods of the invention is the reversion of progressive loss over generations of certain silencing loci which can sometimes be observed, by introduction into a dcl3 background. The methods of the invention can thus be used to increase the stability of silencing loci in host cells, particularly in plant cells.

It will be clear that the invention also relates to modifying the gene-silencing effect achieved in eukaryotic cells such as plant cells, by modifying the functional level of more than one Dicer protein.

In one embodiment of the invention, eukaryotic cells are provided wherein the functional level of DCL 3 is decreased and the functional level of DCL4 is increased; in another embodiment eukaryotic cells are provided wherein the functional level of both DCL2 and DCL4 are decreased or increased. Plant cells with a reduced level or functional level of DCL2 and DCL4 protein may be used to increase viral replication in such cells.

In another aspect of the invention, a method is provided for reducing the expression of a target gene in a eukaryotic cell or organism, particularly in a plant cell or plant, comprising the introduction of a silencing RNA encoding chimeric gene, as herein defined, into said cell or organism, characterized in that the cell or organism is modulated in the expression of genes or the functional level of proteins involved in the transcriptional silencing of said silencing RNA encoding chimeric gene.

One example of a class of genes involved in transcriptional silencing are the methyltransferases controlling RNA-directed DNA methylation, such as the MET class, the CMT class and the DRM class (Finnegan and Kovac 2000 Plant Mol. Biol. 43, 189-201, herein incorporated by reference). MET1 in Arabidopsis, like its mammalian homolog Dnmt1 (Bestor et al. 1988, J. Mol. Biol. 203, 971-983) or corresponding genes in other cells encodes a major CpG maintenance methyltransferase (Finnegan et al. 1996, Proc. Natl. Acad. Sci. USA 93, 8449-8454; Ronemus et al. 1996, Science 273, 654-657: Kishimoto et al. Plant Mol. Biol. 46, 171-183). CMT-like genes are specific to the plant kingdom and encode methyltransferase proteins containing a chromodomain (Henikoff and Cornai, 1998, Genetics 149, 307-318). The DRM genes share homology with mammalian Dnmt3 genes that encode de novo methyltransferases (Can et al. 2000, Proc; Natl. Acad. Sci. USA 97, 4979-4984).

Methods to reduce or inactivate the expression of methyltransferases are as described elsewhere in this document concerning the Dicer-like proteins. The nucleotide sequences and amino acid sequences of methyltransferases in plants are known and include N_{—}177135, AAK69756, AAK71870. AAK69757; NP_{—}199727, NP_{—}001059052 and others (herein incorporated by reference). Methods to identify the endogenous homologues of the above mentioned specific methyltransferases and encoding genes are known in the art, and may be used to identify nucleic acids encoding proteins having at least 50%, 60%, 70%, 80%, 90%, 95% sequence identity with the above mentioned amino acid sequences, variants thereof as well as mutant, less or non-functional variants thereof.

Another class of genes involved in transcriptional silencing includes the RDR2 (RNA dependent polymerase) genes and poIIV (DNA polymerase IV) genes (also named NRPD1a/SDE4 and NRDP2a) (Elmayan et al. 2005, Current Biology 15, 1919-1925 and references therein). The amino acid sequences for these proteins are known and include NP_{—}192851 and ABL68089 (herein incorporated by reference). Methods to identify the endogenous homologues of the above mentioned specific polymerases and encoding genes are known in the art and may be used to identify nucleic acids encoding proteins having at least 50%, 60%, 70%, 80%, 90%, 95% sequence identity with the above mentioned amino acid sequences, variants thereof as well as mutant, less or non-functional variants thereof.

Having read the exemplified embodiments with hpRNA silencing RNA, the skilled person will immediately realize that similar effect can be achieved using other types of silencing RNA artificially introduced into a host cell/plant cell, whereby the processing in siRNA molecules involves a double stranded RNA phase, including conventional sense RNA, antisense RNA, unpolyadenylated RNA, end RNA wherein the silencing RNA includes largely double stranded regions comprising a nuclear localization signal from a viroid of the Potato spindle tuber viroid-type or comprising MG trinucleotide repeats as described e.g. in WO 03/076619 WO04/073390 WO99/53050 or WO01/12824.

An enzymatic assay which can be used for detecting RNAse III enzymatic activity is described e.g; in Lamontagne et al., Mol Cell Biol. 2000 February; 20(4): 1104-1115. The resulting cleavage products can be further analyzed according to standard methods in the art for the generation of 21-24 nt siRNAs.

It is also an object of the invention to provide host cells, plant cells and plants containing the chimeric genes or mutant alleles according to the invention. Gametes, seeds, embryos, either zygotic or somatic, progeny or hybrids of plants comprising the chimeric genes or mutant alleles of the present invention, which are produced by traditional breeding methods are also included within the scope of the present invention. Also encompassed by the invention are plant parts from the herein described plants, such as leaves, stems, roots, fruits, stamen, carpels, seeds, grains, flowers, petals, sepals, flower primordial, cultured tissues and the like.

The methods and means described herein are believed to be suitable for all plant cells and plants, gymnosperms and angiosperms, both dicotyledonous and monocotyledonous plant cells and plants including but not limited to Arabidopsis, alfalfa, barley, bean, corn or maize, cotton, flax, oat, pea, rape, rice, rye, safflower, sorghum, soybean, sunflower, tobacco and other Nicotiana species, including Nicotiana benthamiana, wheat, asparagus, beet, broccoli, cabbage, carrot, cauliflower, celery, cucumber, eggplant, lettuce, onion, oilseed rape such as canola or other Brassicas, pepper, potato, pumpkin, radish, spinach, squash, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon, Brassica vegetables, sugarcane, vegetables (including chicory, lettuce, tomato) and sugarbeet. For some embodiments of the invention, the plant cell could be a plant cell different from an Arabidopsis cell, or the plant could be different from Arabidopsis.

The methods according to the invention, particularly the increase of the functional level of DCL3 or DCL4 protein may also be applicable to other eukaryotic cells, e.g. by introduction of a chimeric gene expressing DCL4 into such eukaryotic cells. The eukaryotic cell or organism may also be a fungus, yeast or mold or an animal cell or organism such as a non-human mammal, fish, cattle, goat, pig, sheep, rodent, hamster, mouse, rat, guinea pig, rabbit, primate, nematode, shellfish, prawn, crab, lobster, insect, fruit fly, Coleopteran insect, Dipteran insect, Lepidopteran insect or Homeopteran insect cell or organism, or a human cell. Eukaryotic cells according to the invention may be isolated cells; cells in tissue culture; in vivo, ex vivo or in vitro cells; or cells in non-human eukaryotic organisms. Also encompassed are non-human eukaryotic organisms which consist essentially of the eukaryotic cells according to the invention.

Introduction of chimeric genes (or RNA molecules) into the host cell can be accomplished by a variety of methods including calcium phosphate transfection, DEAE-dextran mediated transfection, electroporation, microprojectile bombardment, microinjection into nuclei and the like.

Methods for the introduction of chimeric genes into plants are well known in the art and include Agrobacterium-mediated transformation, particle gun delivery, microinjection, electroporation of intact cells, polyethyleneglycol-mediated protoplast transformation, electroporation of protoplasts, liposome-mediated transformation, silicon-whiskers mediated transformation etc. The transformed cells obtained in this way may then be regenerated into mature fertile plants, and may be propagated to provide progeny, seeds, leaves, roots, stems, flowers or other plant parts comprising the chimeric genes.

A “transgenic plant”, “transgenic cell” or variations thereof refers to a plant or cell that contains a chimeric gene (“transgene”) not found in a wild type plant or cell of the same species. A “transgene” as referred to herein has the normal meaning in the art of biotechnology and includes a genetic sequence which has been produced or altered by recombinant DNA or RNA technology and which has been introduced into the cell. The transgene may include genetic sequences derived from the same species of cell. Typically, the transgene has been introduced into the plant by human manipulation such as, for example, by transformation but any method can be used as one of skill in the art recognizes.

Transgenic animals can be produced by the injection of the chimeric genes into the pronucleus of a fertilized oocyte, by transplantation of cells, preferably undifferentiated cells into a developing embryo to produce a chimeric embryo, transplantation of a nucleus from a recombinant cell into an enucleated embryo or activated oocyte and the like. Methods for the production of transgenic animals are well established in the art and include U.S. Pat. No. 4,873,191; Rudolph et al. 1999 (Trends Biotechnology 17:367-374); Dalrymple et al. (1998) Biotechnol. Genet. Eng. Rev. 15: 33-49; Colman (1998) Bioch. Soc. Symp. 63: 141-147; Wilmut et al. (1997) Nature 385: 810-813, Wilmute et al. (1998) Reprod. Fertil. Dev. 10: 639-643; Perry et al. (1993) Transgenic Res. 2: 125-133; Hogan et al. Manipulating the Mouse Embryo, 2^{nd} ed. Cold Spring Harbor Laboratory press, 1994 and references cited therein.

Gametes, seeds, embryos, progeny, hybrids of plants or animals comprising the chimeric genes of the present invention, which are produced by traditional breeding methods are also included within the scope of the present invention.

As used herein, “the nucleotide sequence of gene of interest” usually refers to the nucleotide sequence of the DNA strand corresponding in sequence to the nucleotide sequence of the RNA transcribed from such a gene of interest unless specified otherwise.

Mutants in Dicers or Dicer-like proteins, such as DCL3- or DCL4-encoding genes are usually recessive, accordingly it may advantageous to have such mutant genes in homozygous form for the purpose of reducing the functional level of such Dicer proteins. However, it may also be advantageous to have the mutant genes in heterozygous form. Whenever reference is made to a “functional level which is modulated, or increased or decreased with regard to the wild type level” typically, the wild type level refers to the functional or actual level of the corresponding protein in a corresponding organism which is isogenic to the organism in which the modulated functional level is assessed, but for the genetic variation, the latter including presence of a transgene or presence of a mutant allele. Preferably, the “wild type” level in terms of functional level or activity of an enzyme or of a protein refers to the average of the activity of the protein or enzyme in a collection of individuals of a species which are generally recognized in the art as being wild type organisms. Preferably, the collection of individuals consists of at least 6 individuals, but may of course include more individuals such as at least 10, 20, 50, 100 or even 1000 individuals. With regard to an amino acid sequence of a polypeptide or protein, the “wild type” amino acid sequence is preferably considered as the most common sequence of that protein or polypeptide in a collection of individuals of a species which are generally recognized in the art as being wild type organisms. Again preferably the collection of individuals consists of at least 6 individuals. A modulated functional level differs from the wild type functional level preferably by at least 5% or 10% or 15% or 20% or 25% or 30% or 40% or 50% or 60% or 70% or 80% or 90% or 95% or 99%. The modulated functional level may even be a level of protein or enzyme activity which is non-existent or non-detectable for practical purposes. A mutant protein can be considered as a protein which differs in at least one amino acid (e.g. insertion, deletion or substitution) from the wild type sequence as herein defined and which is preferably also altered in activity or function.

It will be clear that the methods as herein described when applied to animal or humans may encompass both therapeutic and non-therapeutic methods and that the chimeric nucleic acids as herein described may be used as predicaments for the purpose of the above mentioned therapeutic methods.

The following Examples describe methods and means for modulating dsRNA mediated silencing of the expression of a target gene in a plant cell by modulating the functional level of proteins involved in processing in siRNA of artificially introduced dsRNA molecules such as DCL3 and DCL4.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

Throughout the description and Examples, reference is made to the following sequences:
SEQ ID No.: 1: amino acid sequence of At_DCL4 (Arabidopsis thaliana).
SEQ ID No.: 2: nucleotide sequence encoding At_DCL4.
SEQ ID No.: 3: amino acid sequence of Pt_DCL4 (Populus trichocarpa).
SEQ ID No.: 4: nucleotide sequence encoding Pt_DCL4.
SEQ ID No.: 5: amino acid sequence of Os_DCL4 (Oryza sativa).
SEQ ID No.: 6: nucleotide sequence encoding Os_DCL4.
SEQ ID No.: 7: amino acid sequence of At_DCL3 (Arabidopsis thaliana).
SEQ ID No.: 8: nucleotide sequence encoding At_DCL3.
SEQ ID No.: 9: amino acid sequence of Pt_DCL3 (Populus trichocarpa).
SEQ ID No.: 10: nucleotide sequence encoding Pt_DCL3.
SEQ ID No.: 11: amino acid sequence of Os_DCL3a (Oryza sativa).
SEQ ID No.: 12: nucleotide sequence encoding Os_DCL3a.
SEQ ID No.: 13: amino acid sequence of Os_DCL3b (Oryza sativa).
SEQ ID No.: 14: nucleotide sequence encoding Os_DCL3b.
SEQ ID No.: 15: oligonucleotide primer for the amplification of fragment 1 of the coding sequence of DCL3.
SEQ ID No.: 16: oligonucleotide primer for the amplification of fragment 1 of the coding sequence of DCL3.
SEQ ID No.: 17: oligonucleotide primer for the amplification of fragment 2 of the coding sequence of DCL3.
SEQ ID No.: 18: oligonucleotide primer for the amplification of fragment 2 of the coding sequence of DCL3.
SEQ ID No.: 19: oligonucleotide primer for the amplification of fragment 3 of the coding sequence of DCL3.
SEQ ID No.: 20: oligonucleotide primer for the amplification of fragment 3 of the coding sequence of DCL3.
SEQ ID No.: 21: oligonucleotide primer for the amplification of fragment 4 of the coding sequence of DCL3.
SEQ ID No.: 22: oligonucleotide primer for the amplification of fragment 4 of the coding sequence of DCL3.
SEQ ID No.: 23: oligonucleotide primer for the amplification of fragment 1 of the coding sequence of DCL4.
SEQ ID No.: 24: oligonucleotide primer for the amplification of fragment 1 of the coding sequence of DCL 4.
SEQ ID No.: 25: oligonucleotide primer for the amplification of fragment 2 of the coding sequence of DCL4.
SEQ ID No.: 26: oligonucleotide primer for the amplification of fragment 2 of the coding sequence of DCL4.
SEQ ID No.: 27: oligonucleotide primer for the amplification of fragment 3 of the coding sequence of DCL4.
SEQ ID No.: 28: oligonucleotide primer for the amplification of fragment 3 of the coding sequence of DCL4.
SEQ ID No.: 29: oligonucleotide primer for the amplification of fragment 4 of the coding sequence of DCL4.
SEQ ID No.: 30: oligonucleotide primer for the amplification of fragment 4 of the coding sequence of DCL4.
SEQ ID No.: 31: forward oligonucleotide primer for diagnostic PCR amplification of DCL3.
SEQ ID No.: 32: reverse oligonucleotide primer for diagnostic PCR amplification of DCL3.
SEQ ID No.: 33: forward oligonucleotide primer for diagnostic PCR amplification of DCL4.
SEQ ID No.: 34: reverse oligonucleotide primer for diagnostic PCR amplification of DCL4.
SEQ ID No.: 35: forward oligonucleotide primer for diagnostic PCR amplification of DCL3A.
SEQ ID No.: 36: reverse oligonucleotide primer for diagnostic PCR amplification of DCL3A.
SEQ ID No.: 37: forward oligonucleotide primer for diagnostic PCR amplification of DCL3B.
SEQ ID No.: 38: reverse oligonucleotide primer for diagnostic PCR amplification of DCL3B.

### REFERENCES

An et al., 1996 The Plant Cell 8, 15-30
Blane, G. & Wolfe, K. H. (2004) Plant Cell 16, 1679-1691.
Colman (1998) Bioch. Soc. Symp. 63: 141-147
Dalrymple et al. (1998) Biotechnol. Genet. Eng. Rev. 15: 33-49
Fire et al., 1998 Nature 391, 806-811
Gasciolli et al., 2005 Current Biology, 15, 1494-1500).
Hamilton et al. 1998 Plant J. 15: 737-746
Hapster et al., 1988 Mol. Gen. Genet. 212, 182-190
Hausmann, 1976 Current Topics in Microbiology and Immunology, 75: 77-109
Hedges, S. B, Blair, J. E., Venturi, M. L. & Shoe, J. L. BMC Evol. Biol. (2004) 4:2 1471-2148/4/2
Henikoff et al. Plant Physiol. 2000 June; 123(2):439-42.
Hogan et al. Manipulating the Mouse Embryo, 2^{nd} ed. Cold Spring Harbor Laboratory press, 1994 and references cited therein.
Hudspeth et al., 1989 Plant Mol Biol 12: 579-589
Keil et al., 1989 EMBO J. 8: 1323-1330
Keller et al., 1988 EMBO J. 7: 3625-3633
Keller et al., 1989 Genes Devel. 3: 1639-1646
Kurihara and Watanabe, 2004, Proc. Natl. Mad. Sci. USA 101: 12753-12758).
Lamontagne et al. Mol Cell Biol. 2000 February; 20(4): 1104-1115
Lee et al., 2004 Cell 75:843-854
Needleman and Wunsch 1970
Peleman et al., 1989 Gene 84: 359-369
Perry at al. (1993) Transgenic Res. 2: 125-133
Pham et al., 2004 Cell 117: 83-94.
Qi et al., 2005 Molecular Cell, 19, 421-428
Rudolph et al. 1999 (Trends Biotechnology 17:367-374)
Smith et al., 2000 Nature 407: 319-320
Sterek, L., Rombauts, S., Jansson, S., Sterky, F., Rouze. P. & Van de Peer, Y. (2005) New Phytol. 167, 165-170 Waterhouse et al. 1998 Proc. Natl. Acad. Sci. USA 95: 13959-13964.
Wilmut et al. (1997) Nature 385: 810-813
Wilmute et al. (1998) Reprod. Feral. Dev. 10: 639-643
Xie et al., 2004, PLosBiology, 2004, 2, 642-652).
Yoshikawa et al., 2005, Genes & Development, 19: 2164-2175).

### EXAMPLES

### Example 1

### Identification of Different Dicer Types in Plants

### 1.1 INTRODUCTION

Eukaryotes possess a mechanism that generates small RNAs and uses them to regulate gene expression at the transcriptional or post-transcriptional level (1). These 21-24 nt small RNAs are defined as micro (mi) RNAs, which are produced from partially self-complementary precursor RNAs, or small interfering (si) RNAs, which are generated from double stranded (ds) RNAs (1, 2). The large RNase III-like enzymes that cleave these templates into small RNAs are called Dicer or Dicer-like (DCL) proteins (3), Humans, mice and nematodes each possess only one Dicer gene, yet regulate their development through miRNAs, modify their chromatin state through siRNAs, and are competent to enact siRNA-mediated RNA interference (RNAi) (1, 4). Insects, such as Drosophila melanogaster, and fungi, such as Neurospora crassa and Magnaporthe oryzae, each possess two Dicer genes (4, 5). In Drosophila, the two Dicers have related but different roles: one processes miRNAs and the other is necessary for RNAi (6). In plants, the picture is not clear. It has been reported that rice (Oryza sativa) has two DCL genes, although this was before the complete rice genome had been sequenced, while Arabidopsis thaliana has four (4). Analysis of insertion mutants of the four A. thaliana DCL (AtDCL) genes has revealed that the role of a small RNA appears to be governed by the type of DCL enzyme that generated it: AtDCL1 generates miRNAs, AtDCL2 generates siRNAs associated with virus defense, AtDCL3 generates siRNAs that guide chromatin modification, and AtDCL4 generates trans-acting siRNAs that regulate vegetative phase change (7-40). In this study, we sought to identify whether most plants were like rice, fungi and insects in having two Dicers, or were like Arabidopsis with multiple divergent Dicers. We found evidence suggesting that it is advantageous for plants to have a set of four Dicer types, and that these have evolved by gene duplication after the divergence of animals from plants. The number of Dicer-like genes has continued to increase in plants over evolutionary time, whereas in mammals, the number has decreased. These opposite trends are probably a reflection of the differing threats and defense strategies that apply to plants and mammals. Mammals have immune, interferon and ADAR systems to protect them against invaders, and may only need a Dicer to process miRNAs. Plants have none of these defense systems and, therefore, rely on Dicers to not only regulate their development through miRNAs, but also to defend them against a multitude of viruses and transposons.

### 1.2 MATERIALS AND METHODS

### 1.2.1 Plant Material, PCR Amplification and Sequencing

RNA was extracted from leaf material of the Columbia ecotype of Arabidopsis thaliana using the TRIzol reagent (Invitrogen), reverse transcribed, amplified and cloned into pGEM-T Easy using the OneStep RT-PCR Kit (Quiagen) and pGEM-T Easy vector system 1 kit (Promega). The inserts were sequenced using BigDye terminator cycle sequencing ready reaction kits (PE Applied Biosystems, CA, USA). Amplification reaction conditions for detection of orthologous genes were 35 cycles at 95° C. for 30 see, 52° C. for 30 sec and 72° C. for 1 minute. DNA samples of rice, maize, cotton, lupin, barley and Triticum tauchii were kind girls from Narayana Upadhyaya, Qing Liu and Evans Lagudah. PCR products were separated on a 1.3% agarose gel.

### 1.2.2 Data Collection

The sequences of Arabidopsis, rice, maize, poplar, Chlamydomonas reinhardtii and Tetrahymena genes were accessed via the Arabidopsis Information Resource (TAIR) database (http://www.Arabidopsis.org/index.jsp), the Institute for Genomic Research (TIGR) rice and maize databases (http://www.tigr.org/tigr-scripts/osa1_web/gbrowse/rice; http://tigrblast.tigr.org/tgi_maize/index.cgi), and the JGI Eukaryotic Genomics databases (http://genome.jgi-psf.org/Poptr1/Poptr1.home.html), http://genome.jgi-psf.org/chlre2/chlre2.home.html, and the Tetralrymena genome database http://seq.ciliate.org/cgi-bin/blast-tgd.pl.

### 1.2.3 Sequence Alignment and Phylogenetic Analysis.

Coding sequences of predicted genes were determined by using tBlastn and manual comparison of clustal W-aligned genomic sequences, cDNA sequences and predicted coding sequences (CDS). All protein sequence alignments were made using the program Clustal-W (11). Phylogenetic and molecular evolutionary analyses were conducted using MEGA version 3.1 (12). Trees were generated using the following parameters: complete deletion, Poisson correction, neighbor-joining, Dayhof matrix model for amino acid substitution, and bootstrap with 1000 replications. Protein domains were analysed by scanning protein sequences against the InterPro protein signature database (http://www.ebi.ac.uk/InterProScan) with the InterProScan program (13). Unless otherwise stated, domains were defined according to pFAM predictions (http://www.sanger.ac.uk/Software/Pfam/)

### 1.3 RESULTS AND DISCUSSION

### 1.3.1 Identification of Dicer-Like Genes in Arabidopsis, Poplar and Rice

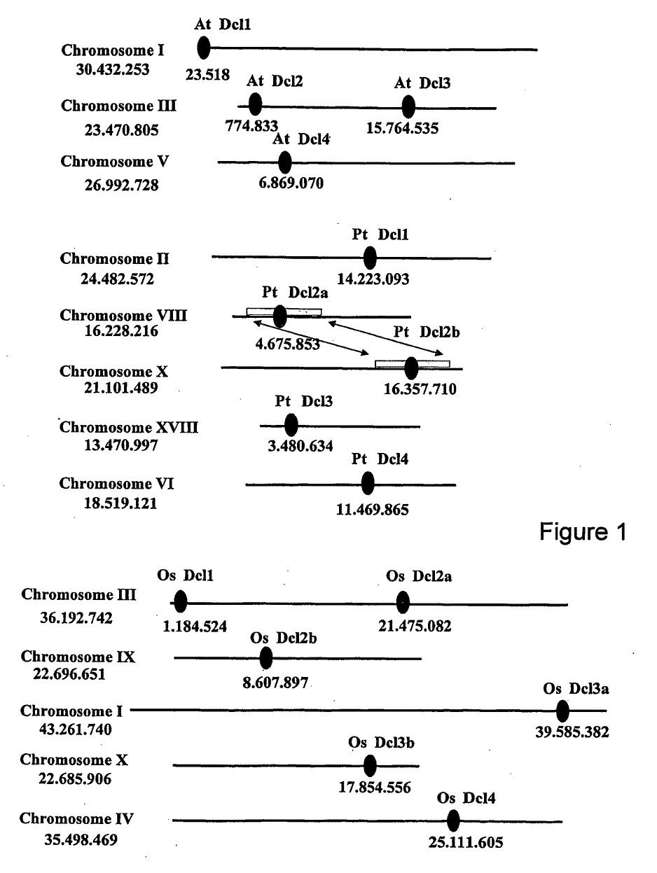

The amino acid sequence of AtDCL1 (At1g01040) has been determined previously by sequencing of cDNAs generated from the gene's mRNA (14). However, the sequences of AtDCL2 (At3g03300), AtDCL3 (At3g43920) and AtDCL4 (At5g20320) have previously been inferred from the chromosomal DNA sequences determined by the Arabidopsis Genome Project (TAIR) using mRNA splicing prediction programs. To obtain more accurate sequences of these proteins, cDNAs were generated from the appropriate Arabidopsis mRNAs, cloned into plasmids and their nucleotide sequences determined. Analysis of these sequences (Genbank accession numbers NM_{—}111200, NM_{—}114260, and NM_{—}122039) showed that the inferred amino acid sequences of AtDCL2, 3 and 4 were largely but not completely correct: at least one exon/intron region has been miscalled for each gene and two different spliceforms of AtDCL2 mRNA were identified. Interrogation of the Arabidopsis genome with the tBLASTn algorithm, using amino acid sequences of each of the DCL sequences, identified no further Dicer-like genes. Repeating essentially the same procedure on the recently completed sequences of the whole genomes of poplar (Populus trichocarpa) and rice (Oryza sativa) revealed five DCL-like genes in poplar (Pt02g14226280, Pt06g11470720, Pt08g4686890, Pt10g16358340, Pt18g3.481550; using the nomenclature in which the number preceding the “g” indicates the chromosome and the number after the “g” indicates the nucleotide position of the start of the coding region on the JGI poplar chromosome pseudomolecules) and six genes in rice (Os01g68120, Os04g43050, Os03g02970, Os03g38740, Os09g14610, Os 10g34430; TIGR build 3 nomenclature). The location of these genes on the genome maps of poplar and rice is shown in FIG. 1.

Phylogenetic analysis, using the PAM-Dayhof matrix model, JTT matrix model, minimum evolution methods and neighbour-joining methods in MEGA 3.1, all showed that the inferred amino acid sequence of each of the rice and poplar DCL proteins strongly aligned with the sequence of an individual member of the four Arabidopsis DCL proteins (FIG. 2A, and pairwise distances in Table 2). With the diversity represented by these plants, from small alpine plant to large tree, and from monocot to dicot, this result suggests that these four types of Dicer are present in all angiosperms and quite possibly all multi-cellular plants. This was further supported by detection of all four genes in barley, maize, cotton and lupin by PCR assays, using primers designed to conserved type-specific sequences (data not shown). We interpreted these groupings to be indicators of orthologous genes, showing that, in, poplar, there are single orthologs of AtDCL1, AtDCL3 and AtDCL4 and a pair of orthologs of AtDCL2, and that in rice, there are single orthologs of AtDCL1 and AtDCL4 and pairs of orthologs of AtDCL2 and AtDCL3. Each gene was named to reflect the species in which it is present, using the prefix Pt or Os, and the number of its Arabidopsis ortholog e.g. PtDCL1. Members of a pair of orthologs were designated A or B with the gene termed A having greater sequence identity to the Arabidopsis ortholog. For all DCL types, the poplar and Arabidopsis orthologs are more similar to each other than to the rice ortholog, as might be expected given that the first two are dicots and rice is a monocot. The Arabidopsis, poplar and rice DCL1 genes group most tightly together, and the second tightest cluster is formed by the DCL4 genes. The DCL2 and DCL3 genes form more expansive clusters showing that they have a higher degree of divergence, and the gene that is the most divergent from the others within the group is OsDCL3B.

### 1.3.2 Correlation of Dicer Type with Domain Variation

Six domain types are present in animal, fungal and plant DCR or DCL proteins, collectively, although many individual proteins lack one or more of them (Table 1). These six types are the DEXH-helicase, helicase-C, Duf283, PAZ, RNaseIII and double stranded RNA-binding (dsRB) domains (4, 15, 16 and references therein). The DEXH and -C domains are found towards the N-terminal and C-terminal regions of the helicase region, respectively. There are always two RNAseIII domains (termed a and b) in a Dicer protein, and the Duf283 is a domain of unknown function but which is strongly conserved among Dicers. The role of the dsRB domain in human Dicer is generally thought to mediate unspecific reactions with dsRNA, with the PAZ, RNaseIIIa and RNaseIIIb domains being crucial for the recognition and spatial cleavage of dsRNAs into si or miRNA (16). In organisms with only one Dicer, this enzyme, with its associated proteins, is presumably the only generator of si and mi RNAs. In organisms with two or more Dicers, there is probably a division of labour.

Each of the inferred amino acid sequences of the Arabidopsis, poplar and rice DCL1, proteins, along with examples of ciliate, algal, fungal, mammalian and insect DCRs (from previously published information or identified by tBLASTn interrogation of available databases) were analysed using the Interpro suite of algorithms. All six domain types were identified and located (FIG. 2) in all of the plant DCL sequences, except for AtDCL3 and OsDCL2B, which were partially lacking the Duf283 domain. The two most striking results from this analysis were that all of the DCL1, 3 and 4 types in plants have a second dsRB (dsRBb) domain which is completely lacking in non-plant DCRs, and that the PAZ domain is absent in the ciliate, fungal and algal DCRs but detectable in all of the plant DCLs, including all three DCL4s, despite previous reports that this domain is missing in AtDCL4 (4, 15). It has been suggested that the absence of a PAZ domain may play an important role in discriminating which accessory proteins a DCR or DCL interacts with, thereby guiding the recognition of its template (18). The correlation between the absence of miRNAs and presence of only a PAZ-free Dicer in Shizosaccharomycyes pombe, has also led to the suggestion that the PAZ domain may play an important rule in measuring the length of miRNAs. However, the presence of the PAZ domain in all plant Dicer types seems to rule out its presence or absence dictating the function of a DCL in plants. The DUF283 domain is absent in some ciliate and fungal DCL3 and in AtDCL3. However, it is present in all the other plant Dicers, including the DCL3-types in rice and poplar. This, similarly, suggests that its presence or absence does not characterize a Dicer-type or its function in plants.

In Arabidopsis, and probably all plants, the four different Dicer types produce small RNAs that play different roles. Each different type requires specificity in recognising its substrate RNA and the ability to pass the small (s) RNA that it generates to the correct effector complex. Unlike all of the other domains, the dsRBb domain, by its presence, absence or type, is a good candidate for regulating substrate specificity and/or the interaction with associated proteins to direct processed sRNAs to the appropriate effector complex. DCL2 proteins are different from the other Dicer-types by their lack of a dsRBb domain and, with the exception of the variation between the dsRBa domains of DCL1 and 3, the net variation between the pair-wise combinations of Dicer-types 1, 3 and 4 is most variable in this domain (FIG. 2 and Table 1). There is good evidence that dsRB domains not only bind to dsRNA but also function as protein-protein interaction domains (21, 22, 23). Indeed, it has been shown that fusion proteins containing both the dsRBa and dsRBb domains of AtDCL1, AtDCL3 and AtDCL4 can bind to members of the HYL1/DRB family of proteins that are probably associated with sRNA pathways in Arabidopsis (23). The simplest model seems be that the dsRBa domain along with the PAZ and RNaseIII a and b domains recognize and process the substrate RNA, while the dsRBb domain specifically interacts with one or two of the different HYL1/DRB members to direct the newly generated sRNAs to their appropriate RNA-cleaving or DNA-methylating/histone-modifying effector complexes (24).

### 1.3.3 DCL Paralogs in Poplar and Rice and Other Gramineae

In both poplar and rice, the DCL2 gene has been duplicated. The paralogs in poplar, PtDCL2A and PtDCL213, have 85% sequence similarity at the amino acid level and are located on chromosomes 8 and 10, respectively. They are within large duplicated Hoax (FIG. 1) that are predicted to have formed during a large scale gene duplication event 8 to 13 million years ago (mya) (19, 25). The timing for this duplication of DCL2 in poplar is consistent with the lack of a DCL2B in Arabidopsis, since the common ancestor of Arabidopsis and poplar is estimated to have existed about 90 mya (20).

The paralogs, OsDCL2A and OsDCL2B, in rice have almost identical sequences (99% sequence similarity at the amino acid level), except for a ˜200 bp deletion, largely within an intron, but also deleting part of the Duf 283 domain in OsDCL2B, which may possibly abolish or impair the protein's function. Apart from this deletion, there are less than 100 nt variations in a genomic sequence of, 14.5 kb. This suggests that the gene duplication occurred relatively recently. Applying the unsophisticated approach of using the rate of amino acid changes that occurred between PtDCL2A and PtDCL2B during the ˜10 million years (my) since their duplication as a measure of time (˜20 aa changes/my), the ˜15 amino acid difference between OsDCL2A and OsDCL2B suggest that this duplication occurred about 1 mya. It has been estimated that the rice subspecies indica and japonica last shared a common ancestor ˜0.44 mya (26). To test whether the duplication event occurred before or after this divergence, DNA extracted from japonica and indica was assayed by PCR using primers, flanking the OsDCL2B deletion. The assay (FIG. 3) showed that both OsDCL2A and OsDCL2B are present in both subspecies, hence placing the duplication event that created them before this time. Examination of the regions surrounding these genes on rice chromosomes 3 and 9 suggest that the duplication was of a relatively small region of chromatin (50-100 kb).

The DCL3 paralogs, OsDCL3A and OsDCL3B, in rice are highly divergent, showing about 57% similarity at the amino acid level. Therefore, the duplication event which created them probably occurred before the generation of PtDCL2A and PtDCL2B in poplar (˜10 mya). However, there is no pair of DCL3 paralogs in either poplar or Arabidopsis, suggesting that the event that produced the OsDCL3 paralog pair occurred after the divergence of monocotyledonous plants from dicotyledonous plants (about 200 mya). In an attempt to refine the estimation of the date when the OsDCL3 paralogs were generated, we sought to determine if they existed before the divergence of maize and rice (˜50 mya). Therefore, the TIGR Release 4.0 of assembled Zea mays (AZM) and singleton sequences was searched for both OsDCL3A-like and OsDCL3B-like sequences. Three sequences were identified, two of which (AZM4_{—}67726 and PUDDE51TD) have greater similarity to OsDCL3B and one (AZM4_{—}120675) which has greater similarity to OsDCL3A. Fortunately, one of the OsDCL3B-like clones (AZM4_{—}67726) covered the same helicase-C domain region as the OsDCL3A-like clone. Phylogenetic analysis (FIG. 4A) showed that these clones grouped as orthologs of OsDCL3A and OsDCL3B, strongly suggesting that the duplication event that generated the DCL3 paralogs occurred before the divergence of maize from rice. Examination of the aligned helicase-C sequences of all of the Arabidopsis, poplar, and rice DCL gene sequences and the two maize clones allowed two sets of primers to be designed that, when used in PCR assays with maize or rice DNA, should discriminate between the DCL3A and DCL3B paralogs in either species and may also be similarly effective in other cereals. Fortunately, the polymorphisms that allowed the design of these discriminating primers are in sequences that flank an intron that is smaller in the OsDCL3A gene than in the OsDCL3B gene (but not in the equivalent genes in maize), thus providing a visible control for the specificity of the amplification products. Using these primer pairs on DNA from rice, maize, and two other diploid cereals, barley (Hordeum vulgare) and Triticum tauchii, a progenitor of wheat, (FIG. 4B), showed that orthologs of both OsDCL3A and OsDCL3B could be detected in all of these species. The PCR products from barley and T. tauchii were cloned and sequenced, which were then compared with the DCL3 Hel-C sequences represented in FIG. 4A. The sequences amplified from barley and T. tauchii with the 3A-specific primers clustered with the OsDCL3A and AZm467726 sequences, and the sequences amplified with the 3B-specific primers clustered with OsDCL311 and AZm467726 (data not shown). This demonstrates that the DCL3 duplication occurred not only before the common ancestor of maize and rice, but also before the common ancestor of barley and rice (˜60 mya).

### 1.3.4 A Fifth Dicer Type in Monocots

The OsDCL3B gene in rice is transcribed, as we could detect its sequence in EST clones (RSICEK_{—}13981 and CK062710)), and has no premature stop codons, suggesting that it is translated into a functional protein. However, this protein has 57% amino acid sequence identity with that of OsDCL3A, showing that the gene has diverged significantly from its paralog, although it has retained the landmark amino acids that give it the domain hallmarks of a functional Dicer. Furthermore, its dsRB domain, which probably governs the role of the small RNAs that the enzyme generates, is highly divergent from all of the other Dicers, showing no phylogenetic grouping with any of them (FIG. 3B). As the DCL3 B gene is present in all of the monocots that we tested, and probably has a specificity different from that of its paralog OsDCL3A, which groups well with PtDCL3 and AtDCL3, we suggest that it has probably evolved to perform a different function. The highly divergent dsRBb may allow it to interact with proteins other than those interacting with the other four Dicer types. Alternatively, this peptide region may be non-functional and thereby give the protein a characteristic similar to the DCL2s. If so, it is possible that it is a case of convergent evolution that increases the plant's ability to combat viruses. Whatever its function, OsDCL3B and its counterparts in other monocots have been retained for over 60 my suggesting that they confer advantage. We suggest that since the gene is highly likely to have a different function to other DCL3 types, it and its counterparts should be considered a different form of Dicer, DCL5.

### 1.3.5 The Origin of Plant Dicers

Examination of the genome of the green algae, Chlamydomonas reinhardtii, which diverged from plants ˜955 mya (27), revealed a single DCR-like gene (C_{—}130110 chlre2/scaffold_{—}13:93930-105880) encoding a protein with single helicase-C, a DUF283 and dsRB domains, and two RNAseIII domains. This initially suggested that the four DCL types in plants have evolved from a single common gene that was present in the common ancestor of algae and plants. However, examining the genuine of the ciliate, Tetrahymena thermophila, which shared a last common ancestor with plants ˜2 billion years ago (27), revealed that there are two DCR-like genes (AB182479 and AB182480 and (ref 28)) which both possess helicase domains and two RNase III domains (FIG. 2). Searching the available genomes of Archaebacteria and Eubacteria, we were unable to identify any protein containing two adjacent RNAseIII domains. In an attempt to discover whether one (and which one) or both of the Tetrahymena genes were the progenitors of animal and plant Dicers, the two RNAseIII domains of both these genes were compared with the RNaseIIIa and b domains of DCRs or DCLs of a nematode, an insect and three plant species. The result (FIG. 5) shows that with the exception of the Tetrahymena domains, all RNaseIIIa domains cluster together and all RNAseIIIb domains cluster together. However, the Tetrahymena RNaseIII a and b domains from DCR1 and DCR2 are more similar to themselves than to either of the RNAseIIIa or RNAseIIIb domain groupings of plants, nematodes and insects. This is an interesting dichotomy of conservation. Insects, nematodes and plants shared a common ancestor about ˜1.6 billion years ago and the phylogenetic tree in FIG. 6 suggests that duplication and distinction into RNAseIIIa and b domains had been well established at this point, and that these differences have been largely conserved since then. Unfortunately, because the Tetrahymena RNAseIIIa and h domains, form an out-group from the domains of the other species, it does not shed light on which one (or both) of the Tetrahymena DCR-like genes is the modern day representative of the progenitor of plant and animal Dicers. However, the simplest model is that the Tetrahymena DCR-like genes were derived from a very ancient duplication, that this pair has been maintained in some fungi and insects, and that in plants the pair has undergone a further duplication. In nematodes, mammals, and other organisms which possess only one Dicer, it appears that they have lost one of the original progenitor genes. FIG. 7 presents a summary of the different Dicer-like genes described in this study, in the context of the evolutionary history of plants, algae, fungi and animals, and predicted events of large scale gene duplication that have occurred in plants. It seems likely that the gene duplication from two to four plant DCL genes that occurred between 955 and 200 mya, the generation of OsDCL3B between 200 and 60 mya, and the generation of PtDCL2B, occurred during the large scale gene duplication events that have been mapped to ˜270, ˜70 and ˜10 mya, respectively (20).

### 1.4 REFERENCES

1, Finnegan, E. J. & Matzke, M. A. (2003) J. Cell Sci. 116, 4689-4693.
2. Bartel, D. (2004) Cell 116, 281-297.
3. Bernstein, E., Caudy, A., Hammond, S. & Hannon, G. (2001) Nature 409, 363-366.
4. Schauer, S., Jacobsen, S., Meinke, D. & Ray. A. (2002) Trends Plant Sci. 7, 487-491.
5. Catalanotto, C., Pallotta, M., ReFalo, P., Sachs, M. S., Vayssie, L., Macino, G. & Cogoni, C. (2004) Mol. Cell. Biol. 24, 2536-2545.
6. Lee, Y. S., Nakahara, K., Pham, J. W., Kim, K., He, Z., Sontheimer, E. J. & Carthew, R. W. (2004) Cell 117, 69-81
7. Park, W., Li, J., Song, R., Messing, J. & Chen, X. (2002) Curr. Biol. 12, 1484-1495.
8. Xie, Z., Johansen, L. K., Gustafson, A. M., Kasschau, K. D., Lellis, A. D., Zilberman, D., Jacobsen, S. E. & Carrington, J. C. (2004) PLoS Biol. 2, E104.
9. Gasciolli, V., Mallory, A. C., Bartel, D. P. & Vaucheret, H. (2005) Curr. Biol. 15, 1494-1500.
10. Xie, Z., Allen, E., Wilken, A. & Carrington, J. C. (2005) Proc. Natl. Acad. Sci. USA 102, 12984-12989.
11. Thompson, J. D., Higgins, D. G. & Gibson, T. J. (1994) Nucl. Acids Res. 22, 4673-4680.
12. Kumar, S., Tamura, K. and Nei, M. (2004) Bioinformatics 5, 150-163.
13. Zdobnov, E. M. & Apweiler, R. (2001) Bioinformatics 17, 847-848.
14. Golden, T. A., Schauer, S. E., Lang, J. D., Pien, S., Mushegian, A. R., Grossniklaus, U., Meinke, D. W. & Ray, A. (2002) Plant. Physiol. 130, 808-822.
15. Finnegan, E. J., Margis, R. & Waterhouse, P. M. (2003) Curr. Biol. 13, 236-240.
16. Zhang, H., Kolb, F. A., Jaskiewicz, L., Westhof, E. & Filipowicz, W. (2004) Cell 118, 57-68.
17. Liu, Q., Rand, T. A., Kalidas, S., Du, F., Kim, H. E., Smith, D. P. & Wang, X. (2003) Science 3W, 1921-1925.
18. Carmell, M. A. & Hannon, G. J (2004) Nat. Struct. Mol. Biol. 11, 214-218.
19. Sterek, L., Rombauts, S., Jansson, S., Sterky, F., Rouze, P. & Van de Peer, Y. (2005) New Phytol. 167, 165-170.
20. Blanc, G. & Wolfe, K. H. (2004) Plant Cell 16, 1679-1691.
21. Consentino, G P, Venkatesan, S., Serluca, F C, Green, S R, Matthews, MB, & Sonenberg, N (1995) Proc. Natl. Acad. Sci. USA 92, 9445-9449.
22. Patel, R C, Stanton, P, McMillian, N M, Williams, BR & Sen GC (1995) Proc. Natl. Acad. Sci. USA 92, 8283-8287.
22. Hiraguri A, Itoh R, Kondo N, Nomura Y, Aizawa D, Murai Y, Koiwa H, Seki M, Shinozaki K, & Fukuhara T (2005) Plant Mal Biol. 57 173-88.
24, Meister G. & Tuschl T. (2004) Nature 431, 343-349.
25. Sterek, L., Rombauts, S., Rouze, P. & Van de Peer, Y. (2005) http://bioinformatics.psb.ugent.be/pdf/jste_BBC_{—}2005.pdf
26. Ma, J. & Bennetzen J. L. (2004) Proc. Natl. Acad. Sci. USA 101, 12404-12410.
27. Hedges, S. B, Blair, J. E., Venturi, M. L. & Shoe, J. L. BMC Evol. Biol. (2004) 4:2 1471-2148/4/2
28. Mochizuki, K. & Gorovsky, M. A. (2005) Genes and Development 19, 77-89.

### Example 2

### Demonstration of the Involvement of DCL3 and DCL4 in Transgene Encoded hpRNA Mediated Silencing

A chimeric gene encoding a dsRNA molecule targeted to silence the expression of the phytoene desaturase in Arabidopsis thaliana (PDS-hp) (according to WO99/53050) was introduced into A. thaliana plants with different genetic background., respectively wild-type, homozygous mutants for DCL2, DCL3 or DCL4. Silencing of the PDS gene expression results in photobleaching.

The results of this experiment are shown in FIG. 8. Silencing by the hpRNA encoding transgene of PDS expression was unimpaired in DCL2 or DCL3 mutant background compared to the silencing of PDS gene expression in a wild-type background, but was significantly reduced in a DCL4 mutant background. Unexpectedly, silencing in mutant DCL3 background was significantly increased.

### Example 3

### Overexpression of DCL4 in A. Thaliana and Effect on the Silencing of Different Silencing Loci

Using standard recombinant DNA techniques, a chimeric gene is constructed comprising the following operably linked DNA fragments:
a CaMV 35S promoter region
a DNA region encoding DCL4 from A. thaliana (SEQ ID 1).
A fragment of the 3′ untranslated end from the octopine synthetase gene from Agrobacterium tumefaciens.

This chimeric gene is introduced in a T-DNA vector, between the left and right border sequences from the T-DNA, together with a selectable marker gene providing resistance to e.g. the herbicide phosphinotricin. The T-DNA vector is introduced into Agrobacterium tumefaciens comprising a helper Ti-plasmid. The resulting A. tumefaciens strain is used to introduce the chimeric DCL4 gene in A. thaliana plants using standard A. thaliana transformation techniques.

Plants with different existing gene-silencing loci, particularly weaker silencing loci are crossed with the transgenic plant comprising the chimeric DCL4 gene and progeny is selected comprising both the gene-silencing locus and the chimeric DCL4 gene.

The following gene-silencing loci comprising the following silencing RNA encoding chimeric genes are introduced:
35S-hpCHS: a chimeric gene under control of a CaMV35S promoter which upon transcription yields a hairpin dsRNA construct comprising long complementary sense and antisense regions of the Chalcone synthase coding region (as described in WO 03/076620)
35S-hpEIN2: a chimeric gene under control of a CaMV35S promoter which upon transcription yields a hairpin dsRNA construct comprising long complementary sense and antisense regions of the ethylene insensitive 2 coding region (as described in WO 03/076620.)
35S-GUShp93: a chimeric gene under control of a CaMV35S promoter which upon transcription yields a hairpin dsRNA construct comprising short complementary sense and antisense regions of the GUS coding legion (as described in WO 2004/073390).
AtU6+20-GUShp93: a chimeric gene under control of a PoIIII type promoter which upon transcription yields a hairpin dsRNA construct comprising short complementary sense and antisense regions of the GUS coding region (as described in WO2004/073390)
35S-GUS: a conventional GUS co-suppression construct (note that one of the lines used is a promoter-cosuppressed GFP line).
35S-asEIN2-PSTVd: a chimeric gene under control of a CaMV35S promoter which upon transcription yields an RNA molecule comprising a long antisense region of the ethylene insensitive 2 coding region and further comprising a PTSVd nuclear localization signal (as described in WO 03/076619)

The progeny plants exhibit a stronger silencing of the expression of the respective target gene in the presence of the chimeric DCL4 gene than in the absence thereof.

### Example 4

### Introduction of Different Silencing Loci in a dcl3 Genetic Background

The gene silencing loci mentioned in Example 2 are introduced into A. thalina dcl3 by crossing. The progeny Plants exhibit a stronger silencing of the expression of the respective target gene in the absence of a functional DCL3 protein than in the presence thereof.

### Example 5

### RNAI-Inducing Hairpin RNAs in Plants Act Through the Viral Defense Pathway

The plant species, Arabidopsis thaliana, has four Dicer-like proteins that produce differently-sized small RNAs, which direct a suite of gene-silencing pathways. DCL1 produces miRNAs⁴, DCL2 generates both stress-related natural antisense transcript siRNAs⁵ and siRNAs against at least one virus⁶, DCL3 makes ˜24 nt siRNAs that direct heterochromatin formation⁶, and DCL4 generates both trans-acting siRNAs which regulate some aspects of developmental timing, and siRNAs involved in RNAi⁷⁻⁹. To obtain further detail of the pathways involved in RNAi and virus defense, we examined the size and efficacy/function of small RNAs engendered by a number of RNAi-inducing hpRNAs, two distinct viruses, and a viral satellite RNA in different single and multiple Dcl-mutant Arabidopsis backgrounds. Examination of siRNA profiles from more than 30 different hpRNA constructs in wild-type (Wt) Arabidopsis, targeting either endogenes or transgenes, revealed that the predominant size class is usually ˜21 nt with a smaller proportion of ˜24 nt RNAs. However, the 21/24 nt ratio can vary depending on the construct. To examine hpRNA-derived siRNAs in Dcl mutants, a hpRNA construct (hpPDS), regulated by the rubisco small subunit (SSU) promoter, was made that targeted the phytoene desaturase gene (Pds); silencing Pds causes a photobleached phenotype in plants³. This construct was transformed into Wt plants and into plants that were homozygous mutant for Dcl2, Dcl3 or Dcl4. The primary Wt and dcl2 transformants showed similar degrees of photobleaching, dcl3 transformants exhibited extreme photobleaching, and dcl4 transformants were mildly photobleached (FIG. 8). The mild silencing in dcl4 indicates that DCL4 activity is important, but not essential, for RNAi. To further test this, the dcl4 line (dcl4-1) and a different mutant line (dcl4-2) were transformed with an hpRNA construct targeting the chalcone synthase (Chs) gene. CHS is required for anthocyanin production; silencing the gene reduces the production of red/brown pigment in the hypocotyls of young seedlings and in the seed coat³. Approximately 30% of the dcl4-1 and 20% of the dcl4-2 plant lines transformed with hpCHS had green hypocotyls and yielded pale seed, affirming that DCL4 activity is not essential for RNAi. In dcl3 plants, hpPDS produced stronger photobleaching than in Wt, showing that DCL3 activity is not required for RNAi. Indeed, its absence appears to enhance silencing. Therefore, we investigated whether DCL2 was processing hpRNA into RNAi-mediating siRNAs in the absence of DCL4.

A construct (hpGFP), containing a green fluorescent protein (GFP) gone and an hpRNA transgene against GFP, was transformed into dcl4-1 and dcl4-1/dcl2 lines. No primary hpGFP/dcl4-1 transformants showed any GFP expression but 5 primary hpGFP/dcl4-1/dcl2 transformants expressed GFP. This suggested that RNAi can operate in the absence DMA, but not in the absence of both DCL4 and DCL2. To examine this further, a crossing strategy was undertaken. A hpPDS/dcl2 line was crossed with dcl4-2 to produce a double heterozygous plant which had also inherited hpPDS. This was self-pollinated to produce progeny that were germinated on media, selective for inheritance of the hpPDS construct, and monitored for symptoms of photobleaching. Most of the seedlings exhibited photobleaching, but a few were unbleached. Genotyping the unbleached seedlings revealed that they were double homozygous dcl2/dcl4-2. Seedlings with any of the other possible genotype combinations exhibited a degree of photobleaching similar to that of the parental hpPDS/dcl2 line, except for a small number which had slightly less severe photobleaching and were homozygous dcl4-2 in combination with either heterozygous Dcl2 or wild-type. The levels of Pds mRNA and hpPDS siRNA profiles were examined in the different genotypes. There were 21 and 24 nt siRNAs in both Wt and dcl2, 22 and 24 nt siRNAs in dcl4-2 and only 24 nt siRNAs detectable in dcl2-dcl4-2. These results suggest that the 24 nt siRNAs have no role in directing mRNA degradation, that 21 nt siRNAs are produced by DCL4 and are the major component directing the mRNA degradation, and that DCL2 (especially in the absence of DCL4) produces 22 nt siRNAs that can also direct mRNA degradation.

To examine the roles of the differently-sized siRNAs in defending plants against viruses, the range of Dcl mutants was challenged with Turnip mosaic virus (TuMV) and Cucumber mosaic virus (CMV), with or without its satellite RNA (Sat). About 18 days post inoculation (dpi), siRNAs derived from CMV or Sat were readily delectable in Wt Arabidopsis plants. Analysing the Dcl mutants at 18 after infection with CMV, CMV+Sat, or TuMV revealed essentially the same siRNA/Dcl-mutant profiles as were obtained for the hpPDS/Dcl-mutants. Furthermore, the steady-state levels of CMV and Sat genomic RNAs were higher in dcl2-dcl4 than in Wt plants. These results suggested that, in plants, hpRNAs are processed into siRNAs and are used to target RNA degradation by the same enzymes and co factors that are used to recognise and restrain viruses. However, when a triple dcl2-dcl3-dcl4-2 mutant was similarly infected, no siRNAs were detectable and the CMV and Sat genomic RNA levels were even higher. This implies that DCL3 plays a role in restricting viral replication and/or accumulation, and contrasts with the increased, rather than decreased, silencing observed for the hpPDS in dcl3 mutants. To investigate this, dcl3 plants were infected with CMV-Sat and the resulting siRNA profile was compared to that in hpPDS/dcl3. In both cases, the production of 24 nt siRNAs was abolished. This similarity in ˜24 siRNA production, but dichotomous consequences, may be explained by DCL3 cleaving the transient double-stranded replicative form of viral RNA to directly reduce its steady-state level, whereas cleavage of hpRNA stems by DCL3 compromises RNAi by removing substrate that would otherwise be processed by DCL2 and DCL4 into 21 and 22 nt siRNAs, respectively.

If hpRNAs are processed like dsRNA from an invading virus, they may also evoke other virus-like characteristics. It has been well demonstrated that virus-infected cells in a plant are able to generate and transmit a long-distance specific signal to uninfected cells thereby triggering a silencing-like response which defends against virus spread⁹. It has also been shown that viruses contain suppressor proteins that suppress the virus defense response¹⁰. Therefore, we conducted grafting experiments to test whether hpRNAs are processed to produce such a signal, and whether RNAi directed by hpRNAs could be prevented by the transgenic expression of the viral suppressor protein HC-Pro¹¹⁻¹². Scions from a tobacco plant expressing a GUS reporter gene were grafted onto rootstocks from plants transformed with an anti-GUS hpRNA construct, and scions from Arabidopsis plants expressing GFP were grafted onto rootstocks transformed with an anti-GFP hpRNA construct. In both systems, the reporter gene in the newly-developing tissues of the scion was silenced. Tobacco plants containing an anti-Potato virus Y construct (hpPVY) and sibling plants also expressing HC-Pro were analysed for their response to inoculation with PVY. The plants containing hpPVY were protected against PVY whereas plants containing the same construct in the Hc-Pro background were susceptible to the virus. Both sets of results further show that hpRNAs are processed by the viral defense pathway.

### REFERENCES FOR EXAMPLE 5

1. Vaucheret, H. (2006) Post-transcriptional small RNA pathways in plants: mechanisms and regulations. Genes & Development 20 759-771.
2. Paddison, P. J., Silva, J. M., Conklin, D. S., Schlabach, M., Li, M., Aruleba, S., Balija, V., O'Shaughnessy, A., Gnoj, L., Scobie, K., Chang, K., Westbrook, T., Cleary, M., Sachidanandam, R., McCombie, W. R., Elledge, S. J. and Harmon, G. J. (2004) A resource for large-scale RNA-interference-based screens in mammals. Nature 428, 427-431.
3. Wesley, S. V., Helliwell, C., Smith, N. A., Wang, M-B, Rouse, D., Liu, Q., Gooding. P., Singh, S., Abbott, D., Stoutjesdijk, P., Robinson, S., Gleave A., Green, A. and Waterhouse, P. M. (2001) Constructs for Efficient, Effective and High Throughput Gene Silencing in Plants. Plant J. 27, 581-590.
4. Park, W, li, J, Song, R, Messing, J, Chen, X: (2002) CARPEL FACTORY, a Dicer homolog, and HEN1, a novel protein, act in microRNA metabolism in Arabidopsis thaliana. Curr Biol. 12, 1484-1495.
5. Borsani O, Zhu J, Verslues P E, Sunkar R, Zhu J K. (2005) Endogenous siRNAs derived from a pair of natural cis-antisense transcripts regulate salt tolerance in Arabidopsis. Cell 123, 1279-91.
6. Xie, Z., Johansen, L. K., Gustafson, A. M., Kasschau, K. D., Lellis, A. D., Zilberman, D., Jacobsen, S. E. and Carrington, J. C. (2004) Genetic and functional diversification of small RNA pathways in plants. PLoS Biol. B, E104
7. Gasciolli, V., Mallory, A. C., Bartel, D. P. and Vaucheret, H. (2005) Partially redundant functions of Arabidopsis Dicer-like enzymes and a role for DCL4 in producing trans-acting siRNAs. Curr. Biol. 15, 1494-1500.
8. Xie, Allen, E., Wilken, A. and Carrington, J. C. (2005) Dicer-LIKE 4 functions in trans-acting small interfering RNA biogenesis and vegetative phase change in Arabidopsis thaliana. Proc. Natl Acad. Sci. USA 102, 12984-12989.
9. Dunoyer P, Himber C, Voinnet O. (2006). Dicer-LIKE 4 is required for RNA interference and produces the 21-nucleotide small interfering RNA component of the plant cell-to-cell silencing signal. Nature Genet 37, 1356-1360.
10. Voinnet, O. (2005) Induction and suppression of RNA silencing: insights from viral infections. Nature Rev Genet. 6, 206-220.
11. Mallory, A. K., Ely, L., Smith, T. H., Marathe, R., Anandalakshmi, R., Fagard, M., Vaucheret, H., Pruss, C., Bowman, L. & Vance, V. B. (2001) HC-Pro suppression of transgene silencing eliminates the small RNAs but not transgene methylation or the mobile signal. Plant Cell 13, 571-583.
12. Anandalakshmi, R., Pruss, G. J., Marathe, R., Mallory, A. C., Smith, T. H. & Vance, V. B. (1998) A viral suppressor of gene silencing in plants. Proc. Natl. Acad. Sci. USA 95, 13079-13084.
13. Waterhouse, P. M., Wang, M-B & Lough T. (2001) Gene silencing as an adaptive defense against viruses. Nature 411, 834-842.
14. Reed, J. W., Nagatani, A., Elich, T. D., Fagan, M. and Chary, J. (1994) Phytochrome A and phytochrome B have overlapping but distinct functions in Arabidopsis development. Plant Physiol. 104, 1139-1149.

### Example 6

### Effect of Mutations Affecting Transcriptional Gene Silencing on the Post-Transcriptional Gene Silencing Achieved by Introduced Silencing RNA Encoding Chimeric Genes

Transgenic Arabidopsis plants which when transcribed yield hpRNA comprising EIN2, CHS or PDS specific dsRNA regions were crossed with Arabidopsis lines a having background comprising a mutation in the CMT3 encoding gene and offspring comprising both the transgene and the background mutation have been selected. Alternatively, Arabidopsis plants comprising a background having a mutation in RDR2 were transformed through floral dipping with the above mentioned hpRNA encoding chimeric genes.

FIG. 9 shows the effect of CMT3 mutation on hpRNA-mediated EIN2 and CHS silencing. The length of hypocotyls grown in the dark on ACC containing medium, is generally longer for the F3 hpEIN2 plants with the homozygous cmt3 mutation than with the wild-type background (wt), indicating stronger EIN2 silencing in the cmt3 background. The transgenic plants inside the box have the mutant background, while the transgenic plants outside the box have the wild-type background. In hpCHS containing plants, the seed coat color is significantly lighter for the hpCHS plants with the cmt3 background than with the wild-type background, indicative of stronger CHS silencing in the former transgenic plants.

Arabidopsis plants comprising a 35S-hpPDS transgene and a mutation in RDR2 exhibited more cotyledon and leaf bleaching were significantly more silenced than plants comprising only the 35S-hpPDS transgene.

Both lines of experimentation indicate that a relief of transcriptional silencing through reduction of the functional level of proteins involved in transcriptional silencing enhance the post-transcriptional silencing of the target genes such as EIN2, CHS or PDS, mediated through the introduction of dsRNA encoding chimeric genes targeted to these genes.

TABLE 1
Variation within and between DCLs of Rice, Poplar and Arabidopsis

| | Domain | | | | | | | |
|---|---|---|---|---|---|---|---|---|
| | DexD | Hel-C | Duf283* | PAZ | RIIIa | RIIIb | dsRBa | dsRBb |
| Variation among DCL1s | 13 (2) | 7 (2) | 11 (2) | 18 (2) | 12 (2) | 7 (2) | 7 (2) | 8 (2) |
| Variation among DCL2s | 30 (3) | 28 (3) | 41 (4) | 48 (4) | 50 (5) | 36 (3) | 54 (5) | — |
| Variation among DCL3s | 40 (4) | 25 (3) | 41 (—) | 64 (5) | 30 (3) | 38 (3) | 50 (5) | 50 (5) |
| Variation among DCL4s | 28 (3) | 39 (4) | 46 (4) | 48 (4) | 36 (3) | 54 (5) | 64 (5) | 42 (4) |
| Sites Analyzed/Domain length | 159/172 | 81/81 | 71/86 | 94/165 | 101/148 | 104/114 | 57/61 | 72/73 |
| Var. between DCL1s and DCL2s | 25 (3) | 43 (4) | 32 (3) | 39 (4) | 32 (3) | 26 (3) | 35 (3) | — |
| Vat. between DCL1s and DCL3s | 25 (3) | 41 (4) | 29 (3) | 39 (4) | 31 (3) | 21 (3) | 46 (4) | 43 (4) |
| Var. between DCL1s and DCL4s | 29 (3) | 38 (3) | 32 (3) | 39 (4) | 36 (3) | 23 (3) | 30 (3) | 43 (4) |
| Var. between DCL2s and DCL3s | 19 (3) | 31 (3) | 20 (3) | 25 (3) | 20 (3) | 13 (2) | 23 (3) | — |
| Var. between DCL2s and DCL4s | 21 (3) | 25 (3) | 17 (2) | 22 (3) | 22 (3) | 12 (2) | 9 (2) | — |
| Var. between DCL3s and DCL4s | 18 (2) | 25 (3) | 15 (2) | 27 (3) | 26 (3) | 12 (2) | 14 (2) | 33 (3) |

Variability: Amino acid substitutions/100 sites
*AtDCL3A was removed from group as deletion in this domain meant that its inclusion would drastically reduce the number of sites analysed

TABLE 2
Pairwise distances between DCLS of Rice, Poplar and Arabidopsis

| | | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|---|
| 1. | AtDcl1 | | 0.009 | 0.010 | 0.011 | 0.012 | 0.012 | 0.013 | 0.013 | 0.011 | 0.013 | 0.012 | 0.012 | 0.012 | 0.012 | 0.012 | 0.010 | 0.009 |
| 2. | PtDcl1 | 0.250 | | 0.011 | 0.012 | 0.012 | 0.012 | 0.013 | 0.013 | 0.011 | 0.014 | 0.012 | 0.011 | 0.012 | 0.012 | 0.012 | 0.010 | 0.008 |
| 3. | OsDcl1 | 0.294 | 0.281 | | 0.012 | 0.012 | 0.012 | 0.013 | 0.012 | 0.012 | 0.013 | 0.012 | 0.011 | 0.012 | 0.012 | 0.011 | 0.009 | 0.008 |
| 4. | AtDcl21 | 0.679 | 0.678 | 0.683 | | 0.004 | 0.014 | 0.014 | 0.013 | 0.012 | 0.012 | 0.011 | 0.012 | 0.013 | 0.011 | 0.012 | 0.010 | 0.008 |
| 5. | AtDcl22 | 0.698 | 0.700 | 0.704 | 0.044 | | 0.015 | 0.014 | 0.013 | 0.012 | 0.012 | 0.011 | 0.012 | 0.012 | 0.011 | 0.012 | 0.009 | 0.009 |
| 6. | PtDcl2a | 0.710 | 0.703 | 0.713 | 0.401 | 0.435 | | 0.011 | 0.013 | 0.011 | 0.011 | 0.011 | 0.011 | 0.012 | 0.011 | 0.011 | 0.009 | 0.008 |
| 7. | PlDcl2b | 0.680 | 0.674 | 0.680 | 0.412 | 0.443 | 0.191 | | 0.013 | 0.013 | 0.012 | 0.012 | 0.013 | 0.012 | 0.013 | 0.012 | 0.010 | 0.009 |
| 8. | OsDcl2 | 0.685 | 0.677 | 0.686 | 0.563 | 0.585 | 0.530 | 0.538 | | 0.012 | 0.012 | 0.012 | 0.012 | 0.013 | 0.013 | 0.012 | 0.010 | 0.009 |
| 9. | AtDcl4 | 0.723 | 0.731 | 0.726 | 0.693 | 0.712 | 0.725 | 0.684 | 0.699 | | 0.012 | 0.012 | 0.012 | 0.012 | 0.010 | 0.011 | 0.009 | 0.008 |
| 10. | PtDcl4 | 0.703 | 0.717 | 0.711 | 0.692 | 0.711 | 0.716 | 0.684 | 0.713 | 0.460 | | 0.013 | 0.011 | 0.012 | 0.011 | 0.011 | 0.009 | 0.009 |
| 11. | OsDcl4 | 0.690 | 0.691 | 0.688 | 0.678 | 0.694 | 0.698 | 0.676 | 0.699 | 0.545 | 0.515 | | 0.013 | 0.012 | 0.012 | 0.012 | 0.009 | 0.010 |
| 12. | OsDcl3a | 0.692 | 0.696 | 0.700 | 0.702 | 0.716 | 0.729 | 0.707 | 0.707 | 0.709 | 0.719 | 0.715 | | 0.013 | 0.013 | 0.014 | 0.009 | 0.009 |
| 13. | OsDcl3b | 0.712 | 0.705 | 0.710 | 0.701 | 0.717 | 0.717 | 0.702 | 0.714 | 0.730 | 0.730 | 0.715 | 0.562 | | 0.012 | 0.012 | 0.010 | 0.008 |
| 14. | RtDcl3 | 0.700 | 0.698 | 0.699 | 0.701 | 0.713 | 0.732 | 0.707 | 0.714 | 0.733 | 0.736 | 0.715 | 0.537 | 0.566 | | 0.013 | 0.009 | 0.008 |
| 15. | AtDcl3 | 0.716 | 0.720 | 0.728 | 0.729 | 0.736 | 0.745 | 0.721 | 0.746 | 0.751 | 0.746 | 0.738 | 0.591 | 0.634 | 0.548 | | 0.008 | 0.008 |
| 16. | HsapDcl1 | 0.825 | 0.828 | 0.823 | 0.802 | 0.820 | 0.820 | 0.799 | 0.815 | 0.829 | 0.824 | 0.825 | 0.822 | 0.822 | 0.822 | 0.847 | | 0.009 |
| 17. | OmDcl1 | 0.866 | 0.867 | 0.860 | 0.863 | 0.863 | 0.872 | 0.863 | 0.869 | 0.879 | 0.871 | 0.863 | 0.871 | 0.873 | 0.865 | 0.886 | 0.724 | |

1. Use of a plant or plant cell with a modified functional level of a Dicer protein involved directly or indirectly in processing of artificially provided double-stranded RNA (dsRNA) molecules in short interfering RNA (siRNA) to modify a gene-silencing effect on a target gene or nucleic acid, said gene silencing effect being achieved by the provision of a gene-silencing chimeric gene.

2. Use according to claim 1, wherein the gene-silencing chimeric gene is a gene encoding a silencing RNA, said silencing RNA being selected from a sense RNA, an antisense RNA, an unpolyadenylated sense or antisense RNA, a sense or antisense RNA further comprising a largely double stranded region, hairpin RNA (hpRNA).

3. Use according to claim 1, wherein said Dicer protein is Dicer-like 3 (DCL3) or Dicer-like 4 (DCL4).

4. Use of a plant or plant cell with modified functional level of a Dicer-like 3 protein to modulate the gene-silencing effect obtained by introduction of silencing RNA involving a double stranded RNA during the processing of said silencing RNA into siRNA, such as a daRNA or hpRNA.

5. Use according to claim 4, wherein said modulation of said functional level of said Dicer-like 3 is a decrease in said functional level, and wherein said gene-silencing effect obtained by provision of said silencing RNA is increased compared to a plant wherein said Dicer-like 3 protein level is not modified.

6. Use according to claim 5, wherein said target gene is an endogene or a transgene.

7. Use according to claim 5, wherein said decrease in said functional level is achieved by mutation of said Dicer-like 3 protein encoding endogenous gene.

8. Use according to claim 4, wherein said modulation of said functional level of said Dicer-like 3 is a increase in said functional level, and wherein said gene-silencing effect obtained by introduction of said silencing RNA is decreased compared to a plant wherein said Dicer-like 3 protein level is not modified.

9. Use according to claim 8, wherein said increase in said functional level is achieved by introduction into said plant cell of a chimeric gene comprising the following operably linked DNA regions:
a) a plant-expressible promoter
b) a DNA region encoding a DCL3 protein
c) a transcription termination and polyadenylation region functional in plant cells.

10. Use according to claim 4, wherein said silencing RNA is a dsRNA molecule which is introduced in said plant cell by transcription of a chimeric gene comprising:
a) a plant-expressible promoter
b) a DNA region which when transcribed yields an RNA molecule, said
RNA molecule comprising sense and antisense nucleotide sequence,
i) said sense nucleotide sequence comprising about 19 contiguous nucleotides having about 90 to about 100% sequence identity to a nucleotide sequence of about 19 contiguous nucleotide sequences from the RNA transcribed from a gene of interest comprised within said plant cell;
ii) said antisense nucleotide sequence comprising about 19 contiguous nucleotides having about 90 to 100% sequence identity to the complement of a nucleotide sequence of about 19 contiguous nucleotide sequence of said sense sequence;
wherein said sense and untisense nucleotide sequence are capable of forming a double stranded RNA by basepairing with each other.

11. Use according to claim 5 wherein said chimeric gene is introduced by transformation.

12. Use according to claim 4 wherein said chimeric gene is introduced into said plant with said modified functional level by crossing said plant with a plant comprising said chimeric gene.

13. A method for reducing the expression of a gene of interest in a plant cell, said method comprising the step of providing a silencing RNA molecule into said plant cell wherein processing of said silencing RNA into siRNA comprises a phase involving dsRNA characterized in that said plant cell comprises a functional level of Dicer-like 3 protein which is modified compared to the functional level of said Dicer-like 3 protein in a wild-type plant cell.

14. The method according to claim 13 wherein said method comprises:
a) introducing a dsRNA molecule into a plant cell, said dsRNA molecule comprising a sense and antisense nucleotide sequence,
i) said sense nucleotide sequence comprising about 19 contiguous nucleotides having at least about 90%, such as 94% to about 100% sequence identity to a nucleotide sequence of about 19 contiguous nucleotide sequences from the RNA transcribed from said gene of interest;
ii) said antisense nucleotide sequence comprising about 19 contiguous nucleotides having at 1 east about 90% such as 94% to 100% sequence identity to the complement of a nucleotide sequence of about 19 contiguous nucleotide sequence of said sense sequence;
iii) wherein said sense and antisense nucleotide sequence are capable of forming a double stranded RNA by basepairing with each other.

15. The method according to claim 13, wherein said functional level of Dicer-like 3 protein is reduced by mutation of the endogenous gene encoding said Dicer-like 3 protein of said plant cell.

16. A plant cell comprising a silencing RNA molecule which has been introduced into said plant cell wherein processing of said silencing RNA into siRNA comprises a phase involving dsRNA characterized in that said plant cell further comprises a functional level of dicer-like 3 protein which is different from the wild type functional level of dicer-like 3 protein in said plant cell.

17. The plant cell according to claim 16, wherein said silencing RNA is transcribed from a chimeric gene encoding said silencing RNA.

18. The plant cell according to claim 16, wherein said functional level of Dicer-like 3 protein is decreased.

19. The plant cell according to claim 16, wherein the endogenous gene encoding said Dicer-like 3 protein of said plant has been altered by mutation.

20. A chimeric gene comprising the following operably linked DNA molecules:
a) a plant-expressible promoter
b) a DNA region encoding a Dicer-Like 3 protein
c) a termination transcription and polyadenylation signal which functions in a plant cell.

21. The chimeric gene according to claim 20, wherein said Dicer-like 3 protein is a protein comprising a double stranded binding domain of type 3.

22. The chimeric gene according to claim 21 wherein said double stranded binding domain comprises an amino acid sequence having at least 50% sequence identity to an amino acid sequence selected front the following sequences:
a) the amino acid sequence of SEQ ID NO: 7 (At_DCL3) from the amino acid at position 1436 to the amino acid at position 1563;
b) the amino acid sequence of SEQ ID NO: 11 (OS_DCL3) from the amino acid at position 1507 to the amino acid at position 1643;
c) the amino acid sequence of SEQ ID NO: 13 (OS_DCL3b) from the amino acid at position 1507 to the amino acid at position 1603;
d) the amino acid sequence of SEQ ID NO: 9 (Pt_DCL3a) from the amino acid at position 1561 to the amino acid at position 1669.

23. The chimeric gene according to claim 22, wherein said DCL3 protein has all amino acid sequence having at least 60% sequence identity with the amino acid sequence of SEQ ID NO: 7, 9, 11 or 13.

24. A eukaryotic host cell comprising a chimeric gene according to claim 20.

25. The eukaryotic host cell of claim 24, which is a plant cell.

26. The eukaryotic host cell of claim 24, which is an animal cell.

27. A method for reducing the expression of a gene of interest comprising the step of providing a gene-silencing molecule to a eukaryotic host cell of claim 24.

28. Use of a plant or plant cell with modified functional level of a Dicer-Like 4 protein to modulate the gene-silencing effect obtained by provision of silencing RNA involving a double stranded RNA during the processing of said silencing RNA into siRNA, such as a dsRNA or hpRNA.

29. Use according to claim 28, wherein said modulation of said functional level of said Dicer-like 4 is a decrease in said functional level, and wherein said gene-silencing effect obtained by introduction of said silencing RNA is decreased compared to a plant wherein said Dicer-like 4 protein level is not modified.

30. Use according to claim 29, wherein said decrease in said functional level is achieved by mutation of said Dicer-like 4 protein encoding endogenous gene.

31. Use according to claim 28, wherein said modulation of said functional level of said Dicer-like 4 is a increase in said functional level, and wherein said gene-silencing effect obtained by introduction of said silencing RNA is increased compared to a plant wherein said Dicer-like 4 protein level is not modified.

32. Use according to claim 31, wherein said increase in said functional level is achieved by introduction into said plant cell of a chimeric gene comprising the following operably linked DNA regions: a) a plant-expressible promoter b) a DNA region encoding a DCL4 protein c) a transcription termination and polyadenylation region functional in plant cells.

33. Use according to claim 28, wherein said silencing RNA is a dsRNA molecule which is introduced in said plant cell by transcription of a chimeric gene comprising:
a) a plant-expressible promoter
b) a DNA region which when transcribed yields an RNA molecule, said RNA molecule comprising a sense and antisense nucleotide sequence,
i) said sense nucleotide sequence comprising about 19 contiguous nucleotides having at least about 90%, such as about 94% to about 100% sequence identity to a nucleotide sequence of about 19 contiguous nucleotide sequences from the RNA transcribed from a gene of interest comprised within said plant cell;
ii) said antisense nucleotide sequence comprising about 19 contiguous nucleotides having at least about 90%, such as about 94% to 100% sequence identity to the complement of a nucleotide sequence of about 19 contiguous nucleotide sequence of said sense sequence;
wherein said sense and antisense nucleotide sequence are capable of forming a double stranded RNA by basepairing with each other.

34. Use according to claim 28 wherein said chimeric gene is introduced by transformation.

35. Use according to claim 28 wherein said chimeric gene is introduced into said plant with said modified functional level by crossing said plant with a plant comprising said chimeric gene.

36. A method for reducing the expression of a gene of interest in a plant cell, said method comprising the step of introducing a silencing RNA molecule into said plant cell wherein processing of said silencing RNA into siRNA comprises a phase involving dsRNA wherein said plant cell comprises a functional level of Dicer-like 4 protein which is modified compared to the functional level of said Dicer-like 4 protein in a wild-type plant cell.

37. The method according to claim 36, wherein said method comprises:
a) introducing a silencing RNA which is a dsRNA molecule into a plant cell, said dsRNA molecule comprising a sense and antisense nucleotide sequence,
i) said sense nucleotide sequence comprising about 19 contiguous nucleotides having at least about 90%, such as about 94% to about 100% sequence identity to a nucleotide sequence of about 19 contiguous nucleotide sequences from the RNA transcribed from said gene of interest;
ii) said antisense nucleotide sequence comprising about 19 contiguous nucleotides having at least about 90%, such as about 94%, to 100% sequence identity to the complement of a nucleotide sequence of about 19 contiguous nucleotide sequence of said sense sequence;
iii) wherein said sense and antisense nucleotide sequence are capable of forming a double stranded RNA by basepairing with each other.

38. The method according to claim 36, wherein said functional level of Dicer-like 4 protein is reduced by mutation of the endogenous gene encoding said Dicer-like 4 protein of said plant cell.

39. The method according to claim 36, wherein said functional level of Dicer-like 4 protein is increased by expression of a chimeric gene encoding a DCL4 protein.

40. A plant cell comprising a silencing RNA molecule wherein processing of said silencing RNA into siRNA comprises a phase involving dsRNA characterized in that said plant cell further comprises a functional level of dicer-like 4 protein which is different from the wild type functional level of dicer-like 4 protein in said plant cell.

41. The plant cell according to claim 40, wherein said silencing RNA is transcribed from a chimeric gene encoding said silencing RNA.

42. The plant cell according to claim 40, wherein said functional level of Dicer-like 4 protein is decreased.

43. The plant cell according to claim 42, wherein the endogenous gene encoding said Dicer-like 4 protein of said plant has been altered by mutation.

44. The plant cell according to claim 40, wherein said functional level of Dicer-like 4 protein is increased.

45. The plant cell according to claim 44, wherein said functional level of Dicer-like 4 protein is increased by expression of a chimeric gene encoding a DCL4 protein.

46. A chimeric gene comprising the following operably linked DNA molecules:
a) a plant-expressible promoter
b) a DNA region encoding a Dicer-like 4 protein
c) a termination transcription and polyadenylation signal which functions in a plant cell.

47. The chimeric gene according to claim 46, wherein said Dicer-like 4 protein is a protein comprising a double stranded binding domain of type 4.

48. The chimeric gene according to claim 47 wherein said double stranded binding domain comprises an amino acid sequence having at least 50% sequence identity to an amino acid, sequence selected, from the following sequences:
a) the amino acid, sequence of SEQ ID NO: 1 (At_DCL4) from the amino acid at position 1622 to the amino acid at position 1696;
b) the amino acid sequence of SEQ ID NO: 5 (OS_DCL4) from the amino acid at position 1520 to the amino acid at position 1593; or
c) the amino acid sequence of SEQ ID NO: 3 (Pt_DCL4) from the amino acid at position 1514 to the amino acid at position 1588.

49. The chimeric gene according to claim 46, wherein said DCL4 protein has an amino acid sequence having at least 60% sequence identity with the amino acid sequence of SEQ ID NO: 1, 3 or 5.

50. A eukaryotic host cell comprising a chimeric gene according to claim 46.

51. The eukaryotic host cell of claim 50, which is a plant cell.

52. The eukaryotic host cell of claim 50, which is an animal cell.

53. A method for reducing the expression of a gene of interest comprising the step of providing a gene-silencing molecule to a eukaryotic host cell of claim 50.

54. Use of a eukaryotic cell with a modified functional level of a Dicer protein to reduce the expression of a gene of interest, wherein the gene of interest is silenced in said cell by providing said cell with a gene-silencing molecule.

55. Use according to claim 54, wherein said eukaryotic cell is a cell different from a plant cell, and wherein said functional level of a said Dicer protein is increased.

56. Use according to claim 54, wherein said gene-silencing molecule is an RNA molecule comprising:
a) a nucleotide sequence of at least 19 consecutive nucleotides which has a sequence identity of at least 90% or at least 94% to the nucleotide sequence of said gene of interest; or
b) a nucleotide sequence of at least 19 consecutive nucleotides which has a sequence identity of at least 90% or at least 94% to the complement of the nucleotide sequence of said gene of interest; or
c) a first nucleotide sequence of at least 19 consecutive nucleotides which has a sequence identity of at least 90% or at least 94% to the nucleotide sequence of said gene of interest and a second nucleotide sequence of at least 19 consecutive nucleotides which has a sequence identity of at least 90% or at least 94% to the complement of the nucleotide sequence of said gene of interest, wherein said first and second nucleotide sequence are capable of forming a double stranded RNA region between each other.

57. Use according to claim 54, wherein said RNA molecule is provided to said cell by transcription of a chimeric gene.

58. Use according to claim 54 wherein said RNA molecule is provided to said cell exogenously.

59. Use according to claim 54 wherein said RNA molecule is provided to said cell endogenously.

60. Use of a gene-silencing molecule to reduce the expression of a gene of interest in a eukaryotic cell, characterized in that said eukaryotic cell comprises an altered functional level of a Dicer protein.

61. Use according to claim 60 wherein said eukaryotic cell is a cell different from a plant cell, and wherein said functional level of a said Dicer protein is increased.

62. Use according to claim 61 wherein said gene-silencing molecule is an RNA molecule comprising:
a) a nucleotide sequence of at least 19 consecutive nucleotides which has a sequence identity of at least 90% or at least 94% to the nucleotide sequence of said gene of interest; or
b) a nucleotide sequence of at least 19 consecutive nucleotides which has a sequence identity of at least 90% or at least 94% to the complement of the nucleotide sequence of said gene of interest; or
c) a first nucleotide sequence of at least 19 consecutive nucleotides which has a sequence identity of at least 90% or at least 94% to the nucleotide sequence of said gene of interest and a second nucleotide sequence of at least 19 consecutive nucleotides which has a sequence identity of at least 90% or at least 94% to the complement of the nucleotide sequence of said gene of interest, wherein said first and second nucleotide sequence are capable of forming a double stranded RNA region between each other.

63. Use according to claim 62, wherein said RNA molecule is provided to said cell by transcription of a chimeric gene.

64. Use according to claim 62, wherein said RNA molecule is provided to said cell exogenously.

65. Use according to claim 62, wherein said RNA molecule is provided to said cell endogenously.

66. A eukaryotic cell comprising a double stranded RNA molecule, provided to said cell and a functional level of Dicer protein which is modified compared to the wild-type level of said Dicer protein, wherein said dsRNA molecule reduces the expression of a gene of interest in said cell.

67. The eukaryotic cell of claim 66, wherein said Dicer protein is DCL3 or DCL4.

68. The eukaryotic cell of claim 66, wherein said functional level of Dicer protein is increased.

69. The eukaryotic cell of claim 65, wherein said eukaryotic cell is different from a plant cell and said functional level of Dicer protein is increased.

70. The eukaryotic cell of claim 66, which is a plant cell.

71. The eukaryotic cell of claim 66, wherein said eukaryotic cell is a plant cell and said functional level of Dicer protein is reduced.

72. The eukaryotic cell of claim 66, wherein said dsRNA molecule comprises a first nucleotide sequence of at least 19 consecutive nucleotides which has a sequence identity of at least 90% or at least 94% to the nucleotide sequence of said gene of interest and a second nucleotide sequence of at least 19 consecutive nucleotides which has a sequence identity of at least 90% or at least 94% to the complement of the nucleotide sequence of said gene of interest, wherein said first and second nucleotide sequence are capable of forming a double stranded RNA region between each other.

73. The eukaryotic cell of claim 66, wherein said dsRNA molecule is provided to said cell by transcription of a chimeric gene comprising a promoter functional in said cell operably linked to a DNA region encoding said RNA molecule.

74. The eukaryotic cell of claim 66, wherein said dsRNA molecule is provided exogenously to said cell.

75. A method for the modification of the gene silencing response of a eukaryotic cell comprising providing said cell with a modified functional level of a Dicer protein.

76. The method according to claim 75, wherein said Dicer protein is DCL3 or DCL4.

77. The method according to claim 75, wherein said eukaryotic cell is different from a plant cell and said functional level of a Dicer protein is increased.

78. The method according to claim 75, wherein said eukaryotic cell is from a plant cell which is different from Arabidopsis.

79. The method according to claim 75, wherein said functional level of a Dicer protein is 20 increased.

80. The method according to claim 75, wherein said eukaryotic cell is a plant cell, and said functional level is decreased.

81. The method according to claim 80, wherein said functional level is decreased by mutagenesis.

82. The method according to claim 80, wherein said functional level is decreased by inhibiting said functional level of said Dicer.

83. A eukaryotic cell comprising an increased level of DCL3 or DCL4 protein.

84. A cell, different from an Arabidopsis cell, comprising a modified level of DCL3 or DCL4 protein.

85. The cell of claim 83, wherein said cell has an improved gene silencing phenotype.

86. A method for identifying a cell with a modified functional level of a Dicer protein, comprising the steps of:
a) Screening a population of cells comprising said Dicer protein for the level of a compound in said cell or in an extract of said cell, wherein said level of said compound is directly linked to said functional level of said Dicer protein,
b) identifying those cells within said population wherein the level of said compound is different.

87. The method of claim 86, wherein said population has been subjected to mutagenesis prior to said screening.

88. The method of claim 86, wherein said Dicer protein is DCL3 or DCL4.

89. The method of claim 86, wherein said compound is a nucleic acid such a siRNA of about 21 to 24 nucleotides.

90. The method of claim 86, wherein said compound is said Dicer protein.

91. The method of claim 86 wherein cells of said population comprise a reporter gene, whose expression or function is dependent upon the functional level of said Dicer protein, and said compound is directly related to the expression or function of said reporter gene.

92. A plant cell comprising a reduced level of DCL2 and DCL4.

93. The plant cell of claim 92, further comprising a reduced level of DCL3.

94. Use of the plant cell according to claim 93 to reduce the gene-silencing effect obtained by introducing of a gene-silencing RNA molecule into said plant cell.

95. Use of the plant cell according to claim 92 to increase viral replication in said plant cell.

96. Use of a eukaryotic cell with a modulated functional level of DCL3 to alter the virus resistance of said eukaryotic cell.

97. Use according to claim 96, wherein said virus is a virus having a double stranded RNA intermediate.

98. Use according to claim 96; wherein said level of DCL3 is increased and said virus resistance is increased.

99. Use according to claim 96, wherein said level of DCL3 is decreased and said virus resistance is decreased.

100. A method for reducing the expression of a gene of interest in a eukaryotic cell, said method comprising the step of providing a silencing RNA molecule into said cell by the provision or a silencing RNA encoding chimeric gene wherein processing of said silencing RNA into siRNA comprises a phase involving dsRNA characterized in that said cell comprises a functional level of a protein involved in transcriptional silencing which is modified compared to the functional level of said protein involved in transcriptional silencing in a wild-type cell.

101. The method according to claim 100 wherein said method comprises:
a) introducing a dsRNA molecule into said cell, said dsRNA molecule molecule comprising a sense and antisense nucleotide sequence,
i) said sense nucleotide sequence comprising about 19 contiguous nucleotides having at least about 90%, such as 94% to about 100% sequence identity to a nucleotide sequence of about 19 contiguous nucleotide sequences from the RNA transcribed from said gene of interest;
ii) said antisense nucleotide sequence comprising about 19 contiguous nucleotides having at least about 90% such as 94% to 100% sequence identity to the complement of a nucleotide sequence of about 19 contiguous nucleotide sequence of said sense sequence;
iii) wherein said sense and antisense nucleotide sequence are capable of forming a double stranded RNA by basepairing with each other.

102. The method according to claim 100, wherein said protein involved in transcriptional silencing is a methyltransferase.

103. The method according to claim 102 wherein said methyltransferase is CMT3 or a homologue thereof.

104. The method according to claim 100, wherein said functional level of said protein involved in transcriptional silencing is reduced.

105. The method according to claim 100, wherein said protein involved in transcriptional silencing is selected from RDR2, poIIVa or poIIVb or homologue of any of the preceding proteins.

106. The method according to claim 105, wherein said functional level of said protein involved in transcriptional silencing is reduced.

107. The method according to claim 100, wherein said eukaryotic cell is a plant cell or said eukaryotic organism is a plant.

108. A eukaryotic cell comprising a silencing RNA molecule encoding chimeric gene into said cell wherein processing of said silencing RNA into siRNA comprises a phase involving dsRNA characterized in that said cell comprises a functional level of a protein involved in transcriptional silencing which is modified compared to the functional level of said protein involved in transcriptional silencing in a wild-type cell.

109. The cell according to claim 108 wherein said cell comprises a chimeric gene encoding a silencing RNA molecule said silencing RNA molecule being a dsRNA molecule, said dsRNA molecule comprising a sense and antisense nucleotide sequence,
i) said sense nucleotide sequence comprising about 19 contiguous nucleotides having at least about 90%, such as 94% to about 100% sequence identity to a nucleotide sequence of about 19 contiguous nucleotide sequences from the RNA transcribed from said gene of interest;
ii) said antisense nucleotide sequence comprising about 19 contiguous nucleotides having at least about 90% such as 94% to 100% sequence identity to the complement of a nucleotide sequence of about 19 contiguous nucleotide sequence of said sense sequence;
iii) wherein said sense and antisense nucleotide sequence are capable of forming a double stranded RNA by basepairing with each other,

110. The cell according to claim 108, wherein said protein involved in transcriptional silencing is a methyltransferase.

111. The cell according to claim 110 wherein said methyltransferase is CMT3 or a homologue thereof.

112. The cell according to claim 108, wherein said functional level of said protein involved in transcriptional silencing is reduced.

113. The cell according to claim 108, wherein said protein involved in transcriptional silencing is selected from RDR2, poIIVa or poIIVb or homologue of any of the preceding proteins.

114. The method according to claim 113, wherein said functional level of said protein involved in transcriptional silencing is reduced.

115. The cell according to claim 108, wherein said eukaryotic cell is a plant cell.

116. A non-human eukaryotic organism comprising or consisting essentially of the cells according to claim 108.


SEQUENCE LISTING (38 sequences)

SEQ ID NO: 1 - protein, 1702 aa, Arabidopsis thaliana
MRDEVDLSLTIPSKLLGKRDREQKNCEEEKNKNKKAKKQQKDPILLHTSAATHKFLPPPLTMPYSEIGDDLRSLDFDHADVSSDLHLTSSSSVSSFSSSSSSLFSAAGTDDPSPKMEKDPRKIARRYQVELCKKATEENVIVYLGTGCGKTHIAVMLIYELGHLVLSPKKSVCIFLAPTVALVEQQAKVIADSVNFKVAIHCGGKRIVKSHSEWEREIAANEVLVMTPQILLHNLQHCFIKMECISLLIFDECHHAQQQSNHPYAEIMKVFYKSESLQRPRIFGMTASPVVGKGSFQSENLSKSINSLENLLNAKVYSVESNVQLDGFVSSPLVKVYYYRSALSDASQSTIRYENMLEDIKQRCLASLKLLIDTHQTQTLLSMKRLLKRSHDNLIYTLLNLGLWGAIQAAKIQLNSDHNVQDEPVGKNPKSKICDTYLSMAAEALSSGVAKDENASDLLSLAALKEPLFSRKLVQLIKILSVFRLEPHMKCIIFVNRIVTARTLSCILNNLELLRSWKSDFLVGLSSGLKSMSRRSMETILKRFQSKELNLLVATKVGEEGLDIQTCCLVIRYDLPETVTSFIQSRGRARMPQSEYAFLVDSGNEKEMDLIENFKVNEDRMNLEITYRSSEETCPRLDEELYKVHETGACISGGSSISLLYKYCSRLPHDEFFQPKPEFQFKPVDEFGGTICRITLPANAPISEIESSLLPSTEAAKKDACLKAVHELHNLGVLNDFLLPDSKDEIEDELSDDEFDFDNIKGEGCSRGDLYEMRVPVLFKQKWDPSTSCVNLHSYYIMFVPHPADRIYKKFGFFMKSPLPVEAETMDIDLHLAHQRSVSVKIFPSGVTEFDNDEIRLAELFQEIALKVLFERGELIPDFVPLELQDSSRTSKSTFYLLLPLCLHDGESVISVDWVTIRNCLSSPIFKTPSVLVEDIFPPSGSHLKLANGCWNIDDVKNSLVFTTYSKQFYFVADICHGRNGFSPVKESSTKSHVESIYKLYGVELKHPAQPLLRVKPLCHVRNLLHNRMQTNLEPQELDEYFIEIPPELSHLKIKGLSKDIGSSLSLLPSIMHRMENLLVAIELKHVLSASIPEIAEVSGHRVLEALTTEKCHERLSLERLEVLGDAFLKFAVSRHLFLHHDSLDEGELTRRRSNVVNNSNLCRLAIKKNLQVYIRDQALDPTQFFAFGHPCRVTCDEVASKEVHSLNRDLGILESNTGEIRCSKGHHWLYKKTIADVVEALVGAFLVDSGFKGAVKFLKWIGVNVDFESLQVQDACIASRRYLPLTTRNNLETLENQLDYKFLHKGLLVQAFIHPSYNRHGGGCYQRLEFLGDAVLDYLMTSYFFTVFPKLKPGQLTDLRSLSVNNEALANVAVSFSLKRFLFCESIYLHEVIEDYTNFLASSPLASGQSEGPRCPKVLGDLVESCLGALFLDCGFNLNHVWTMMLSFLDPVKNLSNLQISPIKELIELCQSYKWDREISATKKDGAFTVELKVTKNGCCLTVSATGRNKREGTKKAAQLMITNLKAHENITTSHPLEDVLKNGIRNEAKLIGYNEDPIDVVDLVGLDVENLNILETFGGNSERSSSYVIRRGLPQAPSKTEDRLPQKAIIKAGGPSSKTAKSLLHETCVANCWKPPHFECCEEEGPGHLKSFVYKVILEVEDAPNMTLECYGEARATKKGAAEHAAQAAIWCLKHSGFLC

SEQ ID NO: 2 - DNA, 5109 bp, Arabidopsis thaliana
atgcgtgacgaagttgacttgagcttgaccattccctcgaagcttttggggaagcgagacagagaacaaaaaaattgtgaagaagaaaaaaacaaaaacaaaaaagctaaaaagcagcaaaaggacccaattcttcttcacactagtgctgccactcacaagtttcttcctcctcctttgaccatgccgtacagtgaaatcggcgacgatcttcgctcactcgactttgaccacgccgatgtttcttccgaccttcacctcacttcttcttcctctgtttcttcgttttcctcttcttcgtcttctttgttctccgcggctggtacggatgatccttcaccgaaaatggagaaagaccctagaaaaatcgccaggaggtatcaggtggagctgtgtaagaaagcaacggaggagaacgttattgtatatttgggtacaggttgtgggaagactcacattgcagtgatgcttatatatgagcttggtcatttggttcttagtcccaagaaaagtgtttgtatttttcttgctcccaccgtggctttggtcgaacagcaagccaaggtcatagcggactctgtcaacttcaaagttgcaatacattgtggaggcaagaggattgtgaagagccactcggagtgggagagagagattgcagcgaatgaggttcttgttatgactccacaaatacttctgcataacttacagcactgtttcatcaagatggagtgtatctcccttctaatatttgatgagtgtcaccatgctcaacaacaaagcaaccatccttatgcagaaatcatgaaggttttctataaatcggaaagtttacaacggcctcgaatatttggaatgactgcatctccagttgttggcaaagggtcttttcaatcagagaatttatcgaaaagcattaatagccttgaaaatttgctcaatgccaaggtttattcagtggaaagcaatgtccagctggatggttttgtttcatctcctttagtcaaagtatattattatcggtcagctttaagtgatgcatctcaatcgaccatcagatatgaaaacatgctggaggacatcaaacagcggtgcttggcatcacttaagctgctgattgatactcatcaaacacaaaccctcctaagtatgaaaaggcttctcaaaagatctcatgataatctcatatatactctgctgaatcttggcctctggggagcaatacaggctgctaaaatccaattgaatagtgaccataatgtacaagacgagcctgtgggaaagaatcctaagtcaaagatatgtgatacatatctttctatggctgctgaggccctctcttctggtgttgctaaagatgagaatgcatctgacctcctcagcttagcggcgttgaaggaaccattattctctagaaagctagttcaattgattaagatcctttcggtattcaggctagagccacacatgaaatgtataatatttgtcaatcggattgtgactgcaagaacattgtcatgcatactaaataacttggaactgctacggtcttggaagtctgatttccttgttggacttagttctggactgaagagcatgtcaagaaggagtatggaaacaatacttaaacggttccaatctaaagagctcaatttactggttgccactaaagttggtgaagaaggccttgatattcagacatgctgtcttgtgatccgttatgatttaccagagactgttaccagcttcatacagtccagaggtcgtgctcgaatgcctcagtctgaatatgcgtttctagtggacagcggaaacgagaaagagatggatcttattgaaaattttaaagtaaatgaagatcgaatgaatctagaaattacttacagaagctcagaggaaacttgtcctagacttgatgaggagttatacaaagttcatgagacaggagcttgtatcagtggtggaagcagcatctcccttctctataaatattgttctaggcttccacatgatgaattttttcagcccaagccagagtttcaattcaagcctgttgacgaatttggtggaactatctgtcgcataactttacctgctaatgctcctataagtgaaatcgaaagttcactactaccttcgacagaagctgctaaaaaggatgcttgtctaaaggctgtgcatgagttgcacaacttgggtgtacttaacgattttctgttgccagattccaaggatgaaattgaggacgaattgtcagatgatgaatttgattttgataacatcaaaggtgaaggctgttcacgaggtgacctgtatgagatgcgtgtaccagtcttgtttaaacaaaagtgggatccatctacaagttgtgtcaatcttcattcttactatataatgtttgtgcctcatcccgctgataggatctacaaaaagtttggtttcttcatgaagtcacctcttcccgttgaggctgagactatggatatcgatcttcaccttgctcatcaaagatctgtaagtgtaaagatttttccatcaggggtcacagaattcgacaacgatgagataagactagctgagcttttccaggagattgccctgaaggttctttttgaacggggggagctgatcccggactttgttcccttggaactgcaagactcttctagaacaagcaaatccaccttctaccttcttcttccactctgtctgcatgatggagaaagtgttatatctgtagattgggtgactatcagaaactgcttgtcatcaccaatctttaagactccatctgttttagtggaagatatatttcctccttcgggctctcatttaaagctagcaaatggctgctggaatattgatgatgtgaagaacagcttggtttttacaacctacagtaaacaattttactttgttgctgatatctgccatggaagaaatggtttcagtcctgttaaggaatctagcaccaaaagccatgtggagagcatatataagttgtatggcgtggaactcaagcatcctgcacagccactcttgcgtgtgaaaccactttgtcatgttcggaacttgcttcacaaccgaatgcagacgaatttggaaccacaagaacttgacgaatacttcatagagattcctcccgaactttctcacttaaagataaaaggattatctaaagacatcggaagctcgttatccttgttaccatcaatcatgcatcgtatggagaatttactcgtggctattgaactgaaacatgtgctgtctgcttcgatccctgagatagctgaagtttctggtcacagggtactcgaggcgctcacaacagagaaatgtcatgagcgcctttctcttgaaaggcttgaggtgcttggtgatgcattcctcaagtttgctgttagccgacacctttttctacaccatgatagtcttgatgaaggagagttgactcggagacgctctaacgttgttaacaattccaacttgtgcaggcttgcaataaaaaaaaatctgcaggtctacatccgtgatcaagcattggatcctactcagttctttgcatttggccatccatgcagagtaacctgtgacgaggtagccagtaaagaggttcattccttgaatagggatcttgggatcttggagtcaaatactggtgaaatcagatgtagcaaaggccatcattggttgtacaagaaaacaattgctgatgtggttgaggctcttgtgggagctttcttagttgacagtggcttcaaaggtgctgtgaaatttctgaagtggattggtgtaaatgttgattttgaatccttgcaagtacaagatgcttgtattgcaagcaggcgctacttgcccctcactactcgcaataatttggagacccttgaaaaccagcttgactataagttcctccacaaaggtctacttgtacaagcctttatccatccatcttacaacaggcatggaggaggctgctaccagagattggagtttcttggggatgctgttctggactacttgatgacatcctattttttcacagtcttcccgaaactgaaacctggtcaactgaccgatctaagatctctctcagtaaataatgaggcgctagcaaatgttgctgtcagtttttcgctaaagagatttctattttgcgagtccatttatcttcatgaagttatagaggattataccaatttcctggcatcttccccattggcaagtggacaatctgaaggtccaagatgcccaaaggttcttggtgacttggtagaatcctgtttgggggctcttttcctcgattgtgggttcaacttgaatcatgtctggactatgatgctatcatttctagatccggtcaaaaacttgtctaaccttcagattagtcctataaaagaactgattgaactttgccagtcttacaagtgggatcgggaaatatcagcgacgaaaaaggatggtgcttttactgttgaactaaaagtgaccaagaatggttgttgccttacagtttctgcaactggtcggaacaaaagagagggcacaaaaaaggctgcacagctgatgattacaaacctgaaggctcatgagaacataacaacctcccatccgttggaggatgttctgaagaatggcatccgaaatgaagctaaattaattggctacaatgaagatcctatagatgttgtggatcttgttgggctggacgttgaaaacctaaatatcctagaaacttttggcgggaatagtgaaagaagcagctcatacgtcatcagacgaggtctcccccaagcaccatctaaaacagaagacaggcttcctcaaaaggccatcataaaagcaggtggaccaagcagcaaaaccgcaaaatccctcttgcacgaaacatgtgttgctaactgttggaagccaccacacttcgaatgttgtgaagaggaaggaccaggccacctgaaatcattcgtctacaaggtaatcctggaagttgaagatgcgcccaatatgacattggaatgttatggtgaggctagagcaacgaagaaaggtgcagcagagcacgctgcccaagctgctatatggtgcctcaagcattctggattcctttgctga

SEQ ID NO: 3 - protein, 1594 aa, Populus trichocarpa
MSGGHVTGEHSSLSVGGTNARVVSSSIVGDGEESGSGLQKTEKDPRKMARKYQLELCKKALEENIIVYLGTGCGKTHIAVLLIYEMGHLIRQPQKSACVFLAPTVALVHQQAKVIEDSTDFKVGIYCGKSNRLKTHSSWEKEIEQNEVLVMTPQILLYNLSHSFIKMDLIALLIFDECHHAQVKSGHPYAQIMKVFYKNNDGKLPRIFGMTASPVVGKEKYRERVTSLEILLHHLIRENLPRSINSLENLLDAKVYSVEDKEELECFVASPVIRVYLYGPVANGTSSSYEAYYNILEGVKRQCIVEIGKKTDGNQSLESLRSTKRMLIRMHENIIFCLENLGLWGALQACRILLSGDHSEWNALIEAEGNTSDVSMCDRYLNQATNVFAADCTRDGVTSNVSQVEVLKEPFFSRKLLRLIEILSNFRLQPDMKCIVFVNRIVTARSLSHILQNLKFLTSWKCDFLVGVHSGLKSMSRKTMNVILERFRTGKLNLLLATKVGEEGLDIQTCCLVIRFDLPETVASFIQSRGRARMPQSEYVFLVDSGNQKERDLIEKFKIDEARMNIEICDRTSRETFDSIEEKIYKVHATGASITSGLSISLLQQYCSKLPHDEYFDPKPKFFYFDDSEGTVCHIILPSNAPTHKIVGTPQSSIEVAKKDACLKAIEQLHKLGALSEFLLPQQEDTNELELVSSDSDNCEDKDSRGELREMLVPAVLKESWTELEKPIHLNSYYIEFCPVPEDRIYKQFGLFLKAPLPLEADKMSLELHLARGRSVMTKLVPSGLSKFSTDEITHATNFQELFLKAILDRSEFVHEYVPLGKDALSKSCPTFYLLLPVIFHVSERRVTVDWEIIRRCLSSPVFKNPANAVDKGILPSNDCLQLANGCSSIRDVENSLVYTPHQKKFYFITNIVPEKNGDSPCKGSNTRSHKDHLTTTKFLSKTELQELDEHFVDLAPELCELKIIGFSKDIGSSISLLPSVMHRLENLLVAIELKCILSASFSEGDKVTAHRVLEALTTEKCQERLSLERLETLGDAFLKFAVGRHFFLLHDTLDEGELTRKRSNAVFIRDQPFDPYQFFALGHPCPRICTKESEGTIHSQCGSHVTGQAKGSEVRCSKGHHWLHNKTVSDVVEALIGAFLVDSGFKAAIAFLRWIGIKVDFDDSQVINICQASRTYAMLNPSMDLATLENLLGHQFLYKGLLLQAFVHPSHKNGGEFGVMILQFAMTLMFPPEIGVPWRCFYPKMKPGHLTDLRSVLVNNRAFASVAVDRSFHEYLICDSDALSAATKKFVDFVRTPKSERRLLEGPKCPKVLGDLVESSVGAILLDTGFDLNHIWKIMLSFLDPISSFSNLQINPVRELKELCQSHNWDFEVPASKKGRTFSVDVTLSGKDMNISASASNSNKKEAIRMASEKIYARLKDQGLIPMTNSLEEVLRNSQKMEAKLIGYDETPIDVALDAHGFENSKIQEPFGINCSYEVRDSCPPRFEAVDAWSLSPLDFTGGQPSKVDLGTARSRLREICAANSWKPPSFECCTEEGPSHLKSFTYKVVVEIEEAPEMSFECVGSPQMKKKAAAEDAAEGALWYLKHQRHLS

SEQ ID NO: 4 - DNA, 4785 bp, Populus trichocarpa
atgtctggcggtcatgttactggtgaacatagttctctctccgttggtggtacaaatgctcgtgttgtgtcgtcttcgattgttggtgatggagaggaatccggttctggacttcaaaagactgagaaagacccaagaaaaatggcaagaaagtatcagttggaattatgcaagaaagctctggaagagaatataattgtgtatttggggacaggttgtggcaagactcacattgctgtcctgcttatatacgaaatgggtcacttgataaggcaacctcagaagagtgcttgtgtcttccttgcacccactgttgcccttgttcatcagcaagccaaggttatagaggactctactgatttcaaggttgggatctattgcggaaaatccaatcgattgaagacccactctagctgggaaaaagagattgaacaaaatgaggttcttgtcatgacacctcagatactactgtataacttaagtcacagcttcatcaagatggacttaattgcccttttgatatttgacgagtgtcatcatgctcaagtcaaaagcggtcatccttatgcacaaatcatgaaagtcttctacaaaaataatgatggaaaacttccccgtatctttggcatgaccgcatctccagttgtggggaaagaaaaatatagggaaagagtaacttcccttgaaatcttactccatcacctcattcgagaaaatttaccaagaagcatcaatagtcttgaaaatttacttgatgctaaggtgtattcagttgaagacaaggaagagttggaatgctttgtagcatctcctgtaattagagtatatctgtatggtcctgttgcaaatggcacttctagctcctatgaggcttactataatatacttgagggggtcaagcgccagtgcatagtggaaattggcaagaaaacagatggaaaccaaagtcttgaaagtcttcgaagcacaaaaagaatgctcatcagaatgcatgaaaatatcatattttgtttggaaaatcttggcctttggggagcattgcaggcttgtcgtattcttttgagtggtgatcactctgagtggaatgcattgatagaagcagaagggaatactagtgatgtctccatgtgtgatagatacctaaatcaagctacaaatgtctttgccgctgattgtacaagagatggtgtcacatccaatgtatcacaggtggaggttttaaaggagccatttttctcaagaaagcttttacgcctaattgaaattctttccaacttcaggttacaaccagatatgaaatgtatagtttttgtcaataggattgttactgcaagatcactatcacacatcctacaaaatctgaagtttttaacatcttggaagtgtgattttcttgttggggttcactctggactgaagagtatgtcacgaaagacaatgaatgtcattcttgagaggttccggactggaaagttgaacttactgcttgcaactaaagttggtgaagaaggacttgatattcagacatgctgtcttgtgattcgatttgatcttccagaaactgttgccagctttatacaatcaaggggtcgtgcacgtatgcctcaatctgaatatgtttttttggtggacagtggaaaccaaaaggagagagatttgatagagaaatttaaaatagatgaagctcggatgaatattgaaatatgtgaccgtacatcgagggagacatttgatagtattgaggaaaaaatatataaagttcatgcaactggcgcttccataacttctggattaagcatctcattactgcagcagtattgttcaaaactccctcatgatgagtatttcgaccccaagccaaaattcttttattttgatgattctgaaggaactgtttgccacataatcttaccctccaatgctcccacacacaaaatagtcggtacacctcaatcatcaatagaagttgctaaaaaagatgcttgtctgaaagccattgaacaattgcataaactgggtgcattgagtgagtttcttttgccacaacaagaagacacaaatgagttggagttggtgtcatctgattcagataactgcgaagacaaggattcacgaggagaactacgtgagatgctagttcctgctgttctgaaggaatcgtggactgaattggagaagcctatccaccttaactcttactatattgaattttgtcctgttcctgaagacaggatctataagcagtttggtctttttctgaaggcaccactcccactcgaggctgataaaatgagtcttgaacttcacctggctcgtggtagatctgtgatgacaaagcttgtcccatcaggactctcaaaattcagtacagatgagatcacacatgcaacaaactttcaagagttgtttctcaaggccattctcgatcgatcagaatttgttcatgaatatgttcccttgggaaaggatgcattatctaaatcatgcccaaccttctacctattgcttcctgttatttttcatgtctctgaaaggagagtgactgtagattgggagattatcagacgatgtttatcatctcctgttttcaagaatccagccaatgctgtggacaagggaattcttccttcaaatgattgcttgcaacttgctaatggctgcagtagtatacgtgatgttgagaatagtttggtgtacactccacaccagaaaaaattttacttcattactaacattgttcctgaaaagaatggtgacagtccatgcaaaggttcaaacactcggagtcataaggatcacttaacaacaacaaaatttttgtctaaaacagaattgcaagaactggatgagcactttgttgatttggctcctgagctttgtgagttgaagataataggattctctaaagacattgggagttctatttctctacttccatcagttatgcaccgattggaaaacttgcttgtagccattgaattgaaatgcatattatctgcttcgttctctgaaggagataaagttactgcccatagagttttagaagctctcaccacagagaagtgtcaggaacgtctttctcttgaaagacttgaaactcttggtgatgctttcctcaaatttgctgtcggtcggcatttttttcttttgcatgatacccttgatgaaggggagctaactaggaaacgatcaaatgctgtatttattcgtgatcaaccatttgatccctaccaattttttgctttgggtcatccttgccctagaatttgcaccaaggaatcagaaggaactattcattctcaatgtggaagccatgtgactggccaagcaaagggtagtgaagtcagatgcagcaaaggtcaccattggctacataataaaacagtttctgatgtggttgaagctctcataggagcatttctagttgacagtggctttaaagccgcaattgcatttcttagatggataggtattaaagtggattttgatgattcacaagttatcaatatttgccaagcaagcaggacctatgcaatgcttaatccttccatggaccttgctacccttgaaaatttgctggggcatcagttcctgtataaaggtcttcttctacaggcatttgtacatccttcccacaagaatggaggggaatttggtgttatgatactgcaatttgctatgactttgatgtttccgccagagattggagttccttggagatgcttttatccaaaaatgaaaccaggtcacttgacagatctgagatcagtgttggtgaacaacagggcctttgccagtgtagctgttgatcgatctttccatgaatatcttatctgtgattccgatgccctttctgcggccacaaaaaaatttgtggactttgttagaacacctaaatcagaaaggcgtctgctcgaaggaccaaaatgcccaaaggttcttggtgatttggtagagtcttctgtgggtgccattcttcttgacacgggatttgatttgaaccacatctggaagataatgctatccttcttggatccaatctcaagcttttccaatttgcagataaatcctgtgagggaattaaaagaactttgccagtctcataattgggactttgaggttcctgcatcgaagaagggcaggactttttcagttgatgtgacactgagtggtaaagatatgaacatatctgcttctgcgagcaactcaaataaaaaagaggctattagaatggcttcagaaaaaatatatgctaggctgaaggatcaaggcctcataccaatgactaattctttggaggaggttttaaggaatagccagaagatggaagccaaattgataggatatgatgagacccctatagatgtagctcttgatgcccatgggtttgaaaactcgaagatacaggaaccttttggcatcaattgcagctatgaagtgagagattcttgtccaccccgctttgaagctgttgatgcttggtctctatctccattagatttcactggagggcagcccagtaaagtagaccttggaacagccagatctcgtttgcgtgaaatctgtgcggctaacagttggaaacctccttcgtttgaatgctgcactgaagaaggaccaagtcacttaaagtccttcacttacaaggtggttgtggaaatagaagaagcaccagaaatgagttttgaatgtgttgggtctcctcagatgaaaaagaaagctgcagcagaggatgcagcagaaggggcactctggtacttgaaacatcaacgccacttgtcttga

SEQ ID NO: 5 - protein, 1607 aa, Oryza sativa
MGDAAAAAPAAAAAGPSSTRGEPKDPRTIARKYQLDLCKRAVEENIIVYLGTGCGKTHIAVLLIYELGHLIRKPSREVCIFLAPTIPLVRQQAVVIASSTDFKVQCYYGNGKNSRDHQEWENDMRPRQVLVMTPQILLQSLRHCFIKMNSIALLILDECHHAQPQKRHPYAQIMKEFEEFYNSNSVEKFPRVFGMTASPIIGKGGSNKLNYTKCINSLEELLHAKVCSVDNEELESVVASPDMEVYFYGPVNHSNLTTICIKELDSLKLQSERMLRASLCDFKDSQKKLKSLWRLHENIIFCLQELGSFGALQAARTFLSFDGDKLDRREVDLNGSTSSFAHHYLNGATSILSRNKTDGSHAGSFDLEKLEEPFFSNKFSVLINVLSRYGLQENMKCIVFVKRITVARAISNILQNLKCLEFWKCEFLVGCHSGSKNMSRNKMDAIVQRFSSGEVNLLVATSVGEEGLDIQTCCLVVRFDLPETVASFIQSRGRARMTKSKYVVLLERENQSHEKLLNGYIAGESIMNEEIDSRTSNDMFDCLEENIYQVDNTGASISTACSVSLLHCYCDNLPRDMFFTPSPVFFYIDGIEGIICRLILPPNAAFRQADGQPCLSKDEAKRDACLKACVKLHKLGALTDFLLPGPGSRKNKVSVTNNSSNNKVEDDSLREELHEMLIPAVLKPSGLKLDSLSNLHFYYVKFIPIPEDRRYQMFGLFVINPLPVEAETLQMMLAHKFQEMCLKILLDRSEFTSPHVKLGNDVTLEINSTFYLLLPIKQKCYGDRFMIDWPAVERCLSSPIFKDPIDVSVHASYSSNESLRLLDGIFSKTDVVGSVVFSPHNNIFFFVDGILDEINAWSEHSGATYAEHFKERFRIELSHPEQPLLKAKQIFNLRNLLHNRLPETTESEGRELLEHFVELPPELCSLKVIGFSKDMGSSLSLLPSLMYRLENLLVAIELKDVMLSSFPEASQISASGILEALTTEKCLERISLERFEVLGDAFLKYVVGRHKFITYEGLDEGQLTRRRSDVVNNSHLYELSIRKKLQVYIRDQQFEPTQFFAPGRPCKVVCNTDVEVRLHQMDIHPDNRENCNLRCTRSHHWLHRKVIADVVESLIGAFLVEGGFKAAFAFLHWIGIDVDFNNPALYRVLDSSSINLSLMDYTDIAGLEELIGYKFKHKGLLLQAFVHPSFSQHSGGCYQRLEFLGDAVLEYVITSYLYSTYPDIKPGQITDLRSLAVGNDSLAYAAVEKSIHKHLIKDSNHLTSAISKFEMYVKLSNSEKDLLEEPACPKALGDIVESCIGAVLLDSGFNLNYVWKVMLMLLKPVLTFANMHTNPMRELRELCQCHGFELGLPKPMKADGEYHVKVEVNIKSKIIICTAANRNSKAARKFAAQETLSKLKNYGYKHRNKSLEEILIVARKRESELIGYNEDPIDVEADISVKMKSPHIHEENIPFQNTETSFTRSSKFHNQIIAGSGKHDVNNGRNNQPKLATQSGRLPSEATEKSNKKVYHGDMVHKTARSFLFELCAANYWKPPEFKLCKEEGPSHLRKFTYKVVVEIKGASATLLECHSDGKLQKKAAQEHAAQGALWCLKQLGHLPKEEDVRV

SEQ ID NO: 6 - DNA, 4824 bp, Oryza sativa
atgggcgacgccgccgccgccgccccggcagccgcggcggcggggccgagcagcacgcggggggagccgaaggatccgaggacgatcgcgcgcaagtatcaattggatctctgcaagagggctgtggaggagaacatcatagtgtaccttgggacaggatgcggtaagacgcacattgccgtgctgctgatttatgagcttggtcatctcatccgcaagccaagccgcgaggtctgcatcttccttgccccaaccatcccccttgtacgccagcaagctgtggtgatcgcaagttccaccgatttcaaagttcaatgttattatgggaatggtaaaaactcgagagatcatcaggaatgggagaacgacatgaggccacgtcaggtccttgtaatgactccccaaatattattgcaaagtttgcgtcattgcttcatcaagatgaactcaattgcacttctgatacttgatgagtgccatcatgcacaaccgcaaaaacggcatccatatgcgcaaattatgaaggagtttgaggaattctataatagtaacagtgttgagaaattccctcgggtttttggcatgactgcttcaccaattattgggaaaggtgggtctaataaacttaactacacgaaatgtatcaacagtcttgaggaattacttcatgcaaaggtttgttcagttgataatgaagaacttgaaagtgtggttgcatctcctgatatggaggtgtacttttatggcccagttaatcactctaaccttaccacaatatgcatcaaagagcttgatagcttaaagcttcagagcgagcgcatgctaagagcgagcctatgcgatttcaaggattctcagaagaaactgaagtccttatggaggttgcatgaaaatattattttctgtttgcaagaacttggttctttcggagctctgcaagctgcgaggacctttttgtcctttgatggtgataagctagatagaagggaggtcgatcttaacggcagtacttccagcttcgcacatcactacctgaacggagcaacttctattcttagtcgcaacaaaacagatggttcccacgctggttcatttgaccttgagaagcttgaagaacctttcttctcaaataaattttctgttcttatcaatgttctttcgagatacgggttgcaggaaaacatgaaatgcattgtttttgtgaaaagaataactgttgcaagagcaatatcaaacattctccaaaacttgaagtgtcttgaattttggaaatgtgagtttcttgtgggctgccactcaggatcaaagaacatgtcaaggaataagatggatgctattgttcaaaggttttcttctggtgaggtgaatcttttggttgctacaagcgtaggtgaagagggacttgatattcagacgtgttgtcttgttgtgcgatttgatcttccagaaactgttgctagttttatccaatcaagggggcgtgcccggatgactaaatctaaatacgttgttctcctagagagagaaaatcagtctcatgaaaagttgcttaatggttatattgctggtgaaagcattatgaatgaagagatagactcaagaacttcaaatgatatgtttgattgcctcgaggagaacatttatcaagtggataacaccggtgcttccattagcactgcttgtagtgtatctctactacattgctactgcgacaaccttcctagggatatgttttttactccttccccagtgttcttctacattgatggcatcgagggaataatctgcagactaattcttccaccaaatgctgctttccgtcaagcagatggtcaaccatgtctgtcaaaagatgaagctaagagagatgcatgcttaaaggcatgcgtaaaacttcataaattgggtgctttgacagattttcttctgccgggtccaggttcaagaaagaataaagtatcagtaacaaataattcatcaaacaacaaagttgaagatgatagtctaagagaagagcttcatgagatgttaattcctgcagttctcaaaccttcgggattaaagttggattctttatcgaacttgcatttctactatgttaaatttattcccataccagaagataggcgatatcagatgtttggtctctttgtgatcaatccccttcctgtggaagctgaaacattgcaaatgatgcttgcacacaagtttcaagagatgtgtctgaagattctcctggacagatctgagtttacttcaccccatgttaaattgggcaatgatgttacattagaaatcaattcaacattttaccttctgcttcccatcaagcaaaagtgttatggtgatagatttatgattgactggccagcagttgagcgctgtttatcatcaccgatttttaaagaccctatagatgtgtctgtgcatgcctcatattcatcaaatgagtctctgagacttcttgatggaatcttcagtaaaaccgatgtagttggcagtgtagtctttagtccccacaacaatatctttttctttgttgatggcattctggacgaaataaatgcttggagcgagcacagtggtgcaacttatgcagaacacttcaaggaaaggtttcgtattgagctatcacatcctgaacagccacttttgaaggctaaacagatcttcaacctgcgaaatttgcttcataatcggttaccggagaccacagaatcggagggccgtgaattactagagcacttcgtggagttacctccagaattatgctctttgaaggtcattgggttttcaaaagatatgggcagttccctgtccttgttaccttctttaatgtatcgtttggagaatttgctggtggctattgagttgaaggatgtgatgttatcttcttttccagaggcatctcaaattagtgcttctggtatacttgaagcgcttactactgagaaatgtttggagaggatatctttggagcgatttgaagtcctaggcgatgctttcttgaagtacgtagttggacgtcacaagtttattacatatgaaggacttgatgaagggcaattgaccaggagacgttctgatgtagtgaacaattcacatttatatgagttatcgatcagaaaaaaattgcaggtatacatacgggatcaacagtttgaaccgactcagttctttgctccaggaaggccttgtaaagttgtttgcaatactgacgtggaagtgagactacaccagatggatattcatccagataacagagaaaactgtaacctgagatgtacaaggtcacatcattggctgcataggaaagtgattgcagatgttgtcgaatcgcttattggagcatttcttgttgagggtggattcaaagctgcatttgctttcctgcattggattggaatagacgttgattttaataatccagctctctatagggtattagattcaagctccatcaatttatctcttatggactacactgacattgccgggcttgaagaattgataggttacaagttcaaacataagggtctacttctccaagcattcgtacacccttcatttagtcaacattctggaggctgctaccagagactggagtttcttggagacgctgttttggagtatgtgataacttcgtacctctactctacttatccggatataaaacctggtcaaataacagatctaagatcgttagctgttggtaatgattcgcttgcttatgcggcggttgagaaatctatccataagcatcttataaaggattcaaaccatctcacgtcagcaataagtaaatttgagatgtatgtgaagctctccaattcagagaaagacttgcttgaagaaccagcatgtcccaaggctcttggtgatattgttgaatcttgtattggcgcagtgcttttagattcaggcttcaacctgaactatgtttggaaggtaatgcttatgcttctaaagccagtattgaccttcgccaacatgcacactaatccgatgagagaacttcgagagctttgtcagtgtcatggatttgagttaggccttccgaaacctatgaaagctgatggagagtaccatgtcaaagtagaagttaacataaagagcaaaattatcatttgtactgcagcaaatcggaattcaaaagccgctagaaagtttgctgcacaagaaacactttctaaactgaagaattacggttataagcacagaaacaagtcccttgaggagattttgattgttgccaggaagagggaatcagaactgataggatataatgaagatccaatcgatgttgaggctgacatatctgtaaaaatgaagagtccacatatacatgaagagaacataccttttcaaaatacagaaacatctttcactaggagttctaaattccacaatcaaattatagcagggtctggcaaacatgatgtcaataatggaaggaacaatcaacccaagttggcaacacagagtggtcgtctgcctagtgaagcaacagagaaaagcaataaaaaggtgtatcatggtgacatggtacacaaaacagcaagatcattcctttttgaactatgtgctgcaaattattggaaacctcctgaattcaagttatgtaaagaggaagggccaagccaccttcggaagttcacttacaaggtggttgttgagatcaagggtgcttcggcgacccttttggagtgtcatagcgatggtaagcttcagaagaaggctgcacaagagcatgcggcacaaggggcgctctggtgtctcaagcaacttgggcacctaccaaaagaagaggacgttcgtgtatag

SEQ ID NO: 7 - protein, 1228 aa, Arabidopsis thaliana
MHSSLEPEKMEEGGGSNSLKRKFSEIDGDQNLDSVSSPMMTDSNGSYELKVYEVAKNRNIIAVLGTGIDKSEITKRLIKAMGSSDTDKRLIIFLAPTVNLVKQQCCEIRALVNLKVEEYFGAKGVDKWTSQRWDEEFSKHDVLVMTPQILLDVLRSAFLKLEMVCLLIIDECHHTTGNHPYAKLMKEFYHESTSKPKIFGLTASAVIRKAQVSELERLMDSKIFNPEEREGVEKFATTVKEGPILYNPSPSCSLELKEKLETSHLKFDASLRRLQELGKDSFLNMDNKFETYQKRLSIDYREILHCLDNLGLICAHLAAEVCLEKISDTKGESETYKECSMVCKEFLEDILSTIGVYLPQDDKSLVDLQQNHLSAVISGHVSPKLKELFHLLDSFRGDKQKQCLILVERIITAKVIERFVKKEASLAYLNVLYLTENNPSTNVSAQKMQIEIPDLFQHGKVNLLFITDVVEEGFQVPDCSCMVCFDLPKTMCSYSQSQKHAKQSNSKSIMFLERGNPKQRDHLHDLMRREVLIQDPEAPNLKSCPPPVKNGHGVKEIGSMVIPDSNITVSEEAASTQTMSDPPSRNEQLPPCKKLRLDNNLLQSNGKEKVASSKSKSSSSAAGSKKRKELHGTTCANALSGTWGENIDGATFQAYKFDFCCNISGEVYSSFSLLLESTLAEDVGKVEMDLYLVRKLVKASVSPCGQIRLSQEELVKAKYFQQFFFNGMFGKLFVGSKSQGTKREFLLQTDTSSLWHPAFMFLLLPVETNDLASSATIDWSAINSCASIVEFLKKNSLLDLRDSDGNQCNTSSGQEVLLDDKMEETNLIHFANASSDKNSLEELVVIAIHTGRIYSIVEAVSDSSAMSPFEVDASSGYATYAEYFNKKYGIVLAHPNQPLMKLKQSHHAHNLLVDFNEEMVVKTEPKAGNVRKRKPNIHAHLPPELLARIDVPRAVLKSIYLLPSVMHRLESLMLASQLREEIDCSIDNFSISSTSILEAVTTLTCPESFSMERLELLGDSVLKYVASCHLFLKYPDKDEGQLSRQRQSIISNSNLHRLTTSRKLQGYIRNGAFEPRRWTAPGQFSLFPVPCKCGIDTREVPLDPKFFTENMTIKIGKSCDMGHRWVVSKSVSDCAEALIGAYYVSGGLSASLHMMKWLGIDVDFDPNLVVEAINRVSLRCYIPKEDELIELERKIQHEFSAKFLLKEAITHSSLRESYSYERLEFLGD

SEQ ID NO: 8 - DNA, 3687 bp, Arabidopsis thaliana
atgcattcgtcgttggagccggagaaaatggaggaaggtgggggaagcaattcgcttaagagaaaattctctgaaatcgatggagatcaaaatcttgattccgtctcttctcctatgatgactgactctaatggtagttatgaattgaaagtgtacgaggttgctaagaacaggaacataattgctgttttggggacagggattgataagtcagagatcactaagaggcttatcaaagctatgggttcttctgatacagacaaaagattgataattttcttggccccaactgtgaatcttgttaaacagcaatgctgtgagatcagagcacttgtgaatttgaaagttgaagagtactttggagctaaaggagttgataaatggacatctcagcgctgggatgaggaatttagcaagcacgatgttttagttatgactcctcaaatattattggatgtccttagaagtgcattcctgaaactagagatggtatgtcttctaataatagatgaatgccaccataccactggcaatcatccctatgcgaagttaatgaaggaattctatcacgaatccactagcaaaccgaagatatttggattgactgcgtcagccgtcattagaaaagctcaagtatcagaacttgagagactcatggactcaaagatttttaatcctgaagagcgtgaaggagtggaaaagtttgctacaacggttaaagaaggtccaatattgtataacccatcaccatcctgtagtttggaattgaaagaaaagttagaaacttcacacctcaagtttgatgcttctcttagaaggcttcaagagttgggaaaagacagttttctgaatatggataataagtttgagacatatcaaaagagattgtctatcgactacagagagattttgcattgccttgataatcttggcctgatttgcgcacacttggcggctgaagtctgcttggagaaaatctcagatacgaaaggggaaagtgaaacttataaagaatgctcaatggtgtgcaaggaatttcttgaggatattttatccaccattggggtgtatttgccgcaagatgataagagtctggtagatttgcagcaaaaccatctgtcagcagtaatttctgggcatgtatctccaaagctaaaagaactcttccatctattggattcctttagaggtgacaagcaaaagcagtgccttattttagttgagagaattataactgcgaaagtgatcgaaagattcgttaagaaagaagcctctttggcttaccttaatgtcttgtatttaaccgaaaacaacccctccaccaatgtatcggcacagaaaatgcaaattgaaatccctgatttatttcaacatggcaaggtgaatcttttattcatcacagatgtggttgaagagggatttcaggttccagattgctcatgcatggtttgttttgacctgcccaaaacaatgtgtagttactcgcagtctcaaaaacatgccaaacagagtaattctaagtctatcatgtttcttgaaagagggaacccgaagcaaagagaccatctgcatgaccttatgcgaagagaagtcctaattcaagatccagaagctccaaacttgaaatcgtgtccacctccagtgaaaaatggacacggtgtgaaggagattggatccatggttatcccagattctaacataactgtatctgaggaagcagcttccacacaaactatgagtgatcctcctagcagaaatgagcagttaccaccgtgtaaaaagttacgcttggataacaatctcttacaatccaacggcaaagagaaggttgcctcttctaaaagtaaatcatcttcatcggctgcaggttcaaaaaaacgtaaggagttgcacggaacaacctgtgcaaacgcattgtcaggaacctggggagaaaatattgatggcgccacctttcaggcttataagtttgacttctgttgtaatatttctggcgaagtatactcgagtttctctcttttgcttgagtcaactctcgccgaggatgttggtaaagttgagatggacctttacttggtcaggaagcttgtcaaggcttctgtctcaccttgtggccagatacgtttgagtcaagaggagctggtcaaagcaaaatattttcagcagtttttctttaatggcatgtttggaaagttgtttgttggatctaagtcacagggaacaaagagagaatttttgcttcaaactgacactagttctctttggcaccctgcctttatgtttctactgctaccagttgaaacaaatgatctagcttcgagtgcgacaattgattggtcagctatcaactcctgtgcctcaatagttgagttcttgaagaaaaattctcttcttgatcttcgggatagtgatgggaatcagtgcaatacctcatccggtcaggaagtcttactagacgataaaatggaagaaacgaatctgattcattttgccaatgcttcgtctgataaaaatagtctcgaagaacttgtggtcattgcaattcatactggacggatatactctatagttgaagccgtaagcgattcttctgctatgagcccctttgaggtggatgcctcatcaggctatgctacttatgcagaatattttaacaaaaagtatgggattgttttagcgcacccgaaccagccgttgatgaagttgaagcagagtcaccatgcgcacaaccttttagtcgacttcaatgaagagatggttgtgaagacagaaccaaaagctggcaatgttaggaaaagaaaaccgaatatccatgcgcatttgcctccagagcttttggctagaattgatgtaccgcgtgctgtgctaaaatcaatctacttgctgccttcagtgatgcaccgcctagagtctctaatgttggccagccagcttagggaagagattgattgtagcatagataacttcagtatatcaagtacatcgattcttgaagcagttacaacacttacatgccccgaatcattttcaatggagcggttggaactgctcggggattcagtcttgaagtatgttgcgagctgtcatctattccttaagtatcctgacaaagatgaggggcaactatcacggcagagacaatcgattatatctaactcaaatcttcaccgcttgacaaccagtcgcaaactacagggatacataagaaatggcgcttttgaaccgcgtcgctggactgcacctggtcaattttctctttttcctgttccttgcaagtgtgggattgatactagagaagtaccattggacccaaaattcttcacagaaaacatgactatcaaaataggcaagtcttgcgacatgggtcatagatgggtagtttcaaaatctgtatcagattgcgctgaggccctgattggtgcctattatgtaagcggtggattgtctgcttctctccatatgatgaaatggctcggtattgacgtcgattttgacccaaacctagtcgttgaagccatcaatagagtttctctacggtgttacattcctaaagaagatgagctcatagagttggagagaaagatccaacatgaattctctgcaaagtttcttttaaaagaggctatcacacactcctctcttcgtgaatcctattcatacgagagattagagtttcttggcgattaa

SEQ ID NO: 9 - protein, 1670 aa, Populus trichocarpa
MDTAMPDHDPLKRSFGDMMVNNNSSSSCLAMDTSNGITDHNDTTPQGLASVLSNHKEFYPRGYQSKVFEVAVKRNTIAVLETGAGKTMIAVMLIKQIGQAVFYSGVKRLILFLAPTVHLQYEVIKSQTNFRVGEYYGAKGIDEWSLKSWEKEIDEHDVLVMTPQILLDALRKAFLNLKMVSLLILDECHRSTGNHPYKKIMKDFYHKMENKPKVFGMTASPVVRKGVSSAMDCEDQLAELESVLDSQIYTIEDRAEVHVYVPSAKELCRFYDKAWCSYVELKDKIEASWSKFDASMLALQGSTQSCYKDMDDKLKATRKQLSKDHAKILNCLEDLGLICAYEAIKVCLENAGNPTGECKLYQEISLQCRYFLEDVLHIIGESLLHAEGKERAISYNYKGHRIWINREAREVLCLIFVERIITAKVVERFMKKVEVLAHFTVSYLTGTNASADALAPKMQMETLESFRSGKVNLLFATDVVEEGIHVPNCSCVIRFDLPKTVRSYVQSRGRARQNNSHFITMLERGNTKQRDQLFEIIRSEWSMTDTAINRDPNVWNLKACASEAAKAYVVDVTGASVTADSSVSLIHRYCQHLPGDRYYTPKPTFQFEVFEQSCRCAMKLPPNAAFQTLVGPTCRNQQLAKQLVCLEACKKLHQMGALDDHLLPSVEEPSEIAVVKSKSTSAGAGTTKRKELHGTACIHALSGSWGEKLDGATFHAYKFDFSCSIVSQIYSGFILLIESKLDDDVGNIELDLYLVAKIVKSSISSCGVVHLDAAQMTKAKRFQEFFFNGLFGKLFTGSKSSREFLLQKETTLLWSPSNMYLLLPLEPWSISSNDWCKIDWKGIEACSSVVEYLKNSFLAARSYSGGGNPLPDNVQSSTIECNGTNLIHFANALVNVENIKDMVVLAIHTGRIYSIVKVVNDSSAESAFEGNADNVTEFSTYTEYFNKRLVGPAEGLMFISPRYHLQFPYLTSAALRYGIVLMHPGQPLLRLKQSHNPHNHLVNFNDEGYAVEFSNEFPVLLSDLLHVLLNDQILEAITTLRCCESFSMERLELLGDSVLKYAVSCHLFLKYPNKHEGQLSSWRSGAVCNSTLHKLGTDCKVQVLFNNFADVLRLQKRIPEDFTIEPNVSETKCSAFLTCQGYILDSAFDPRRWAAPGQKSVRTPAPCKCGVDTLEVPLDRKFQTESAIVKVGKPCDSGHRWMGSKTISDCVESVIGAYYVSGGLIAAIHVMKWFGINAELDPSLISEAITSASLRSYIPKEDEIKSLESKLGYTFGVKFVLQEAMTHASIQEQGVTYCYQRLEFLGDSVLDLLITWHLYQSHTDVDPGELTDLRSASVNNDNFAQVAVKQNLYTHLLHCSTLLQSQITEYVNSFHESDQGTKAPKALGDLIESIAGALLIDTKFNLDGVWRIFKPLLSPIVTPEKLELPPLRELVELCDSIGVFVKEKCTKKAEMVHAQLWVQLDNELLSGEGYEKNRKAAKGKAASCLLKKLQKRGIVYSRGGSKRRKQDTDPVVDSSSLGFLESEDFSGKTKPKKQKIENQVPGDSNTDCSPAISPSHGPPVIESINKKKGGPRTSLYDLCKKVQWTMPTFDTTETKSRTAIEFGEGPDKRTGFNSYVSKIIMNIPSYGVVECAGEASADKKTSYDSAALAMLNELEKRGQLIIDESK

SEQ ID NO: 10 - DNA, 5013 bp, Populus trichocarpa
atggatactgcaatgccagaccatgatcctctcaagagaagctttggtgacatgatggtcaacaacaacagctcttcgagctgtcttgctatggatacttccaatggtattactgatcataatgacaccaccccacaaggactagcttctgttcttagtaatcataaggagttttatccaagagggtatcagtcaaaggtttttgaggtggctgttaagaggaatacaattgctgtgctggaaacaggagctggaaagacaatgattgctgttatgttaattaaacagattggtcaagctgtcttttacagtggtgttaagagattgattcttttcttagctccaacagttcatcttcaatatgaagtcattaaatctcaaacaaattttagagtgggagagtactatggagctaagggaatagatgagtggtctctgaagtcctgggagaaggaaattgatgagcacgatgtgttggttatgacaccccagatcctcttggatgccttaagaaaggcatttttgaatctaaaaatggtatccttgttaatacttgacgagtgtcatcgttccaccggtaaccatccttataagaaaattatgaaggatttctatcacaaaatggagaataagccaaaggtttttggaatgacagcatctcctgtagttagaaaaggtgtctcgtctgccatggactgtgaggatcaactagcagaacttgagagtgtattggattctcagatttatactattgaagacagggcagaggtgcatgtctatgttccctctgcaaaagaattatgtagattttatgacaaagcatggtgttcttatgtggagctgaaagataagattgaagcttcatggtccaagtttgatgcttcaatgttagctttgcaaggctcaacacaaagttgttacaaagatatggatgataagcttaaagcaacgagaaagcagttgtccaaggaccacgcaaagattttgaattgcctagaagatcttggcctcatatgtgcttatgaggccatcaaggtttgtctagagaatgctggtaaccccactggtgaatgcaaattatatcaagaaatttctttgcagtgtagatatttccttgaggatgtgttacatataattggtgaatctttgctgcatgctgaggggaaagagagagcgataagttataactataaagggcaccgcatctggataaacagagaagctagggaagtattgtgcctcatttttgttgaaagaattattacagcgaaagtggttgaaagatttatgaagaaagttgaggttttagcacatttcactgtttcatatttgactggaactaatgcatcagctgatgcactggccccaaaaatgcaaatggagaccttggaatcatttcgctctgggaaggtcaatctattatttgccactgatgtggtggaggagggaattcatgtgccaaactgctcctgtgtaatacgttttgatctgcctaagacagtccgcagttatgtccagtctcggggacgagctcgacaaaataattcccactttatcaccatgcttgaaaggggaaacaccaaacaacgggatcagctatttgaaatcattagaagtgagtggtcaatgacagatacagctataaatagagatcctaatgtatggaatctgaaagcatgtgcttcagaagcagcaaaggcttatgtggtggatgtgacaggagcatcagtaactgcagactctagtgttagcctcatacatcgatattgtcaacacctccctggcgacaggtactacacaccaaagccaacttttcagtttgaagtttttgaacagtcttgccgctgtgcaatgaagctacctcctaatgcagcatttcaaacattagttggtccaacatgtaggaatcaacaattagcaaagcagcttgtatgcttggaagcatgtaagaaattgcatcaaatgggtgctttagatgatcatcttctgccatcagttgaagagccttcagaaattgctgttgttaaaagcaagtcaacatctgcaggtgcaggaactacaaaaaggaaggaattgcatgggacagcttgcattcatgcgttatccggaagttggggagagaaacttgatggagccacttttcatgcatacaagtttgatttctcttgctccattgtcagtcagatctattctggatttattcttctcattgagtcaaagctcgatgatgatgtgggaaacattgagttggatctttatttggttgcaaagatagtcaagtcttctatttcttcatgtggagtagttcacttggatgctgcacagatgacaaaagcaaaacggtttcaagaattctttttcaatggcttgtttggaaagttgtttactggatctaaatcatctagggagttcttacttcagaaagaaacaacattactgtggagtccctcaaacatgtatctgcttctaccactagagccatggagcatttccagtaatgattggtgtaaaatagattggaaaggaattgaagcttgctcatctgtggtagaatacttgaagaactcttttttggctgctcggtcttacagtggtggaggaaatccattacctgataatgttcagtcatccaccatagaatgcaatggtacaaatttaatccattttgctaatgctttagtcaacgtagagaacattaaagatatggtggtactggcaatccacacgggaagaatctactccattgttaaagtcgtgaacgattcatctgcggagagtgcttttgagggaaatgctgataatgtgacagagttttctacatacacagagtacttcaacaaaaggttggtgggcccagctgagggtcttatgttcatttctcctcgttatcatctacagtttccatacctaacatcagctgctctcaggtacggaattgtgctgatgcatccaggacagcctctgttgcggttaaagcaaagccataacccacacaatcatcttgtaaactttaatgatgaaggctatgcagttgaatttagcaatgagttccctgttctgttatctgatttgttacatgttctattaaatgaccagattctggaagcgataacaacacttagatgctgtgaaagtttttcgatggagcgactggagttgctaggggactcagttctaaagtatgctgtcagctgccacctatttttaaaatatcccaataaacatgaaggccagttatcctcatggcgctcaggggctgtttgtaattcaaccctacataaattgggaacagattgtaaagtacaggtattgtttaataactttgctgatgttctgcgattgcagaaacgaattccagaagattttacaattgaacccaatgtgtctgaaacaaaatgttctgcatttcttacatgccagggatatatactagacagtgcatttgatccccgtcgttgggctgctcctggacagaaatctgtacgtactcctgctccttgcaaatgtggggttgatactttagaagtaccattggatcgtaagttccaaaccgaaagcgcaattgttaaggttggaaaaccttgtgattcaggccaccgatggatgggttccaaaaccatatcagattgtgttgaatctgtcataggggcatactatgtcagtggtggattgattgctgcaattcatgtgatgaagtggtttggcattaatgctgaacttgatccttcactaataagcgaagcaattacgagtgcatctctacgatcttatatccctaaagaagatgagattaagagtctagagtcaaagcttgggtataccttcggtgtcaagtttgtcttgcaggaggccatgactcatgcatctatacaagaacagggtgttacatactgttaccagaggcttgaatttcttggtgattctgtgttggacttgcttataacatggcatctctatcagagccacacagatgttgatcctggcgagctgactgacttgcgctcagcttctgttaacaatgataactttgctcaagttgctgtgaaacaaaacctatatacccatcttcttcattgttctacactccttcaaagtcaaataacagaatatgtaaattcttttcatgaatctgatcaaggcacaaaggctcccaaggctcttggagacctgattgaaagcattgcaggggcattattaattgatacaaagttcaatctcgatggtgtgtggagaatattcaagcccttgttatctccaattgtaacccctgagaaacttgagctgcctccactgcgtgaacttgttgaattatgtgactctataggggtttttgtaaaagaaaaatgtaccaagaaagctgagatggttcacgcccagctttgggtacagctggacaacgagctcttgtctggagaggggtacgagaagaacaggaaagcagctaaaggaaaagcagcttcttgtttgttgaagaagctccagaaaagaggcatcgtatactcacgtggaggttcaaagaggaggaaacaggacactgaccctgttgttgattcaagttcccttggcttcttagaaagtgaagatttttctggaaaaacaaagccaaaaaagcagaaaatagaaaaccaagtgcccggagattcaaatacagattgttcccctgccatcagtcctagtcatggtcccccagttattgagtcgattaacaagaagaaaggaggacctcgtactagtctttatgatctctgtaagaaagttcaatggacaatgcctacatttgacacaacagaaacgaaatccagaactgcaattgaatttggtgaaggccctgataaaaggacgggatttaacagttatgtatcaaaaatcatcatgaacataccgtcgtatggcgttgttgaatgtgcgggagaggctagtgctgataagaagacctcatatgactctgcggcacttgcaatgcttaatgagcttgaaaaacggggacagctcatcattgatgaatcaaaataa

SEQ ID NO: 11 - protein, 1651 aa, Oryza sativa
MNPLKRSLESSSQEHEAGKQKLQKRECQDFTPRRYQLDVYEVAMRRNTIAMLDTGAGKTMIAVMLIKEFGKINRTKNAGKVIIFLAPTVQLVTQQCEVIEIHTDFEVQQYYGAKGVDQWTGPRWQEQISKYQVMVMTPQVFLQALRNAFLILDMVSLMIFDECHHATGNHPYTRIMKEFYHKSEHKPSVFGMTASPVIRKGISSHLDCEGQFCELENLLDAKIYTVSDREEIEFCVPSAKEMCRYYDSKPVCFEDLSEELGVLCSKYDALITELQNKRSDMYKDADDITKESKRRLSKSIAKICYCLDDVGLICASEATKICIERGQEKGWLKEVVDATDQQTDANGSRLFAENSALHMKFFEEALHLIDKRLQQGIDMLLNSESGCVEAAKTGYISPKLYELIQIFHSFSNSRHARCLIFVDRKITARVIDRMIKKIGHLAHFTVSFLTGGRSSVDALTPKMQKDTLDSFRSGKVNLLFTTDVAEEGIHVPECSCVIRFDLPRTTRTYVQSRGRARQEDSQYILMIERGNVKQNDLISAIVRSETSMVKIASSRESGNLSPGFVPNEEINEYHVGTTGAKVTADSSISIVYRYCEKLPQDKCYSPKPTFEFTHHDDGYVCTLALPPSAVLQILVGPKARNMHKAKQLVCLDACKKLHELGALDDHLCLSVEDPVPEIVSKNKGTGIGTTKRKELHGTTRIHAWSGNWVSKKTALKLQSYKMNFVCDQAGQIYSEFVLLIDATLPDEVATLEIDLYLHDKMVKTSVSSCGLLELDAQQMEQAKLFQGLLFNGLFGKLFTRSKVPNAPREFILNKEDTFVWNTASVYLLLPTNPSFDSNVCINWSVIDAAATAVKLMRRIYSENKRELLGIFDSDQNVGDLIHLANKSCKANSLKDMVVLAVHTGKIYTALDITELSGDSAFDGASDKKECKFRTFAEYFKKKYGIVLRHPSQPLLVLKPSHNPHNLLSSKFRDEGNVVENMSNGTPVVNKTSNRVHMPPELLIPLDLPVEILRSFYLFPALMYRIESLTLASQLRSEIGYSDSNISSFLILEAITTLRCSEDFSMERLELLGDSVLKYAVSCHLFLKFPNKDEGQLSSIRCHMICNATLYKLGIERNVQGYVRDAAFDPRRWLAPGQLSIRPSPCECPVKSEVVTDDIHIIDDKAIVLGKACDKGHRWMCSKTIADCVEAIIGAYYAGGGLRAAMAVLKWLGIGAEIEEDLIVQAILSASVQTYLPKDNVFEMLEAKLGYSFSVKGLLVEALTHPSQQELGAKYCYERLEFLGDAVLDILLTRYLFNSHKDTNEGELTDLRSASVNNENFAQVAVKHNFHHFLQHSSGLLLDQITEYVNRLEGSSMDKVELLSDGLPKGPKVLGDIVESIAGAILLDTKLDLDVVWGIFEPLLSPIVTPENLELPPYRELIEWCGKHGYFVGINCRDQGDTVVATLDVQLKEVLLVRQGFSKKRKDAKAHASSLLLKDLEEKGLIIPKNASKTEQFEKHCGSTNPFNNLHVDAMDTQTPKPTKEKNAADSRNISDPLLGKPLHVIVKTSKGGPRIALYELCKKLQWPMPTMESEKVQPSFSSVCSSPGGSSQKATPQAFAFASTITLHIPNADVISLTGDGRADKKSSQDSAALFLLYEFQRRGTLQLQEV

SEQ ID NO: 12 - DNA, 4956 bp, Oryza sativa
atgaaccctttaaagaggtcattggaatcatcttctcaggaacatgaagcaggcaaacagaaactgcagaagagagagtgtcaagatttcactcccagaagatatcagcttgatgtctatgaggttgcaatgcggagaaacacgattgcgatgcttgacacaggagccgggaagacaatgattgctgtgatgcttatcaaggagttcggaaagataaacagaacaaagaatgctggaaaagtcatcatatttcttgcaccaacagttcaacttgttacacagcaatgcgaggtgattgaaatccacacagattttgaggtacaacaatactatggtgcaaagggggttgatcaatggacaggtcctagatggcaagagcaaatctcaaaataccaggtcatggtcatgacaccacaggtgttcctacaagctttacgcaatgctttcttaatcttggacatggttagtctcatgatatttgatgaatgccatcatgcaactggaaaccacccttatacaagaataatgaaggagttctatcacaaatcagaacataagccaagtgtgtttggtatgacagcatcacctgttataagaaaaggtatctcttctcatttggattgtgaaggtcagttctgtgaattggagaacctgttagatgctaagatctacacagtttcagatagagaagagatagagttttgtgttccttctgcaaaagaaatgtgcaggtactatgactcgaaaccagtttgttttgaagatttgagtgaagaattgggagttttatgttccaagtatgatgcattgataacagagttgcagaataagcgaagcgacatgtataaagatgctgatgatataacaaaagaatcaaagagacgcctttctaaatctatagcaaaaatttgctactgccttgatgatgttggtcttatttgtgcaagtgaggccacaaaaatctgcattgaaaggggccaggagaaaggttggctgaaggaagtagttgatgccacagatcagcaaactgatgcaaatggatcacgcctatttgcagaaaattcagcgcttcatatgaagttctttgaggaagccttgcatttaattgacaaacgcctccaacaaggtatcgacatgcttctaaactcagaaagtggatgtgttgaagcagcaaagacgggctatatttccccaaagctctatgaactcatccagatctttcactcttttagcaactctcgtcatgctcgatgcctcatttttgttgatcgaaagatcactgctagagtcattgaccggatgattaagaaaattggccaccttgcacatttcacagtttcttttcttactggagggagatcttcggtggatgctctgacacccaaaatgcagaaggatacattggattcatttcgctctggaaaggtgaacttactatttactacagatgttgctgaagagggtatccatgtcccagaatgctcttgtgtaatacgatttgatttgccaaggacaacacgtacctatgtgcagtcacgtggacgagcacgccaggaagactctcagtacattctcatgattgaacgggggaacgtgaagcaaaatgatttgatatctgcaattgtgagaagtgagacttcaatggttaagattgcttcaagcagagagtctggaaatctgtcgcctggttttgttcccaatgaagaaataaacgaataccatgtaggcacaacaggagcgaaagtaactgctgattcaagcatcagtattgtctaccgatactgtgagaagcttccgcaggataagtgctactccccaaaacctacatttgagttcactcatcatgatgatggatatgtgtgtacattagcattaccaccaagtgctgtgcttcaaattctggtgggcccaaaagcaagaaacatgcacaaagcaaaacagctcgtttgccttgatgcatgtaagaagttgcatgagctaggagcacttgatgaccacctttgtctatctgttgaagatccagttccagaaattgtaagcaaaaataagggtactggtataggtacaaccaaacggaaggagctacatggtacaacaagaattcatgcttggtctggcaattgggtgtcaaagaaaactgcactcaagcttcaaagctacaaaatgaattttgtttgtgaccaagctggtcagatttactctgaatttgttctgttaattgatgcaactttaccggatgaagtcgctactttggagattgacctatatttgcacgacaagatggtcaaaacttcagtttcttcttgtggacttcttgagttggatgctcaacagatggaacaagcaaagttgtttcaagggcttctcttcaatggtttgtttggaaagctgtttactagatcaaaagtacctaatgctccgagggaattcattcttaataaagaggatacatttgtgtggaacactgcgagtgtatatttgcttttaccaacaaatccttcttttgactccaacgtttgtattaattggagtgtcattgatgcggcagctacggcagttaaacttatgagaaggatttattctgagaataaaagagaattacttggaatatttgattctgaccaaaatgttggagatttaattcatttagctaacaagtcgtgtaaggctaacagcctcaaagatatggtagttctagcagttcacactgggaagatatatactgctcttgatattactgaattatctggcgatagcgcttttgatggtgcatctgataagaaagaatgtaaattccggacattcgcagaatatttcaaaaagaagtatggcatagtacttcgccacccctcacagccactactagttttgaagcctagtcataatcctcacaaccttctttcctcgaagttcagggatgaaggtaatgttgtggagaatatgagtaatggcacaccagttgtaaataaaacaagcaaccgtgtccacatgcctcctgagttgctgattccccttgatttacctgtggaaattttgagatcattctatttgtttccggctttgatgtatcggattgagtcattaacgttagctagtcaactaagaagtgaaattggatacagcgattctaatatatcaagtttcctgattctggaagctattacaacgcttaggtgctctgaggatttctctatggagcgtctagaattattgggagactctgtattgaagtatgcagtgagttgtcatcttttcctgaaatttcctaataaggatgaggggcagctatcatccataaggtgccatatgatttgtaatgccacactttataagcttggaattgaacgcaatgtacagggttacgtacgtgatgctgcatttgatcctcgtcgatggctagcaccaggacagctctctattcgtccatctccttgtgaatgccctgtaaaatctgaggttgtaactgacgatattcatatcattgatgacaaggctattgttctaggcaaggcgtgtgacaagggacacagatggatgtgttccaaaaccattgctgattgtgttgaggctattattggggcatattatgcagggggtggtttaagagcagccatggcagttctcaaatggttgggcatcggggctgaaattgaagaagacttgattgtgcaggccatattgagtgcttctgtgcagacttatcttccaaaagacaatgtatttgaaatgcttgaagcaaaactaggctattctttctcggtgaaaggtcttttggtagaggctctgactcacccatcacagcaggagttaggtgcaaaatactgctacgagcgcctagagttcctcggtgatgcggtcttagacattctgttaacaagatatcttttcaatagtcataaagacactaatgagggggagttgacagacttacgttctgcatcagtcaataatgaaaactttgcacaagttgcagtaaagcacaacttccatcactttctccagcattcttctgggcttctgttagaccaaattactgaatatgtgaataggttggaaggttcatccatggacaaagttgaactgttatcagatggactcccaaaagggcctaaagtccttggtgatattgtagaaagtattgcaggtgcaattcttttagacaccaaacttgatttggatgtagtctggggtatttttgaaccccttctttccccaattgtcacacctgagaatctggagttacctccatacagagagcttatcgaatggtgtggcaaacatgggtattttgtaggaattaactgtagagatcaaggagacacagtagtggctactcttgatgtacagctcaaagaggtgcttcttgtgaggcaaggttttagcaagaaaagaaaagatgcgaaagcgcatgcatcttccttactgctcaaagatctcgaggaaaaaggactaataatcccaaagaatgcaagcaagacagaacaatttgaaaagcattgtggcagcactaatcccttcaacaatttgcatgtcgatgcaatggatacacagactccaaaaccaaccaaggaaaaaaacgcagctgattcaaggaacatttctgatcccctgcttggtaaaccattgcacgtgattgtgaaaacgagtaaaggaggaccacgcattgcattatatgagttgtgtaaaaagttgcaatggccaatgcctacaatggaatctgagaaagtacaaccaagctttagcagcgtgtgctcctcccctggtggttcctctcagaaagctaccccccaagcgttcgctttcgcttcaaccattacattgcatataccaaatgctgatgtgatcagcctcacaggagatggccgtgcagataagaagagctcacaggattctgctgccctgttcttgctctatgagtttcagcggcgaggtactttgcaactccaggaggtgtga

SEQ ID NO: 13 - protein, 1603 aa, Oryza sativa
MADDEAAVLPPPPPLPPPCRPHRQLRPRGSRPTADTTPRTSQLVEVFEAALRGNTIAVLDTGSGKTMVAVMLAREHARRVRAGEAPRRIVVFLAPTVHLVHQQFEVIREYTDLDVMMCSGASRVGEWGADHWKEEVGRNEIVVMTPQILLDALRHAFLTMSAVSLLIFDECHRACGSHPYARIMKIYIVEDRNELESFSPPTTIVNKYYDAYMVDFDNLKSKLQIFSDEFDSLLVGLQESPSNKFKDTDNILETSRKSLSRYHGKILYSLNDLGPIITSEVVKIHIESVKPLCDSEDCIFSKASLCLHMSYFKEALSLIEEILPQGYGELMKSESGSEELTKRGYISSKVNTLINIFKSFGSSNEVLCLIFVDRIITAKAVERFMRGIVNFSCFSISYLTGGSTSKDALSPAVQRFTLDLFRAGKVNLLFTTDVTEEGVDVPNCSCVIRFDLPRTVCSYVQSRGRARRNNSEFILMIERGNLQQQEHIFRMIQTGYYVKNCALYRHPNALSYDLSIQGMYTYQVQSTGATITADCCVNLIRKYCEKLPKDRYFMPKPSFEVTIEDGLFKCTLTLPRNAAFQSIVGPLSSSSNLSKQLVSLEACKKLHQLGELNDHLVPLTEEPMDTDFTTADEKCISGPGTTKRKELHGTTCVLALSGTWIHDSENITLNTYRIDFLCDQEGENYAGFVLLMEPELDDDVAPSKMDLFLIPNKMVYTTVTPRGKVQLNKKQLGKGKLFQEFFFNGIFGRLFHGSRKSGAQRDFIFKKGHEIQWNTESMYLLLPLRDSSYIQDDLSIHWEAIESCAGAVEQLWSSYQGDENVIPVNCIPQKRRGGQEEIIHLANKSLHCSSIKDSVVLSLHTGRIYTVLDLILDTTAEDSFDEMYGSCVLMNFLSSLHCRYGIIIQHPEQPLLLLKQSHNAHNLLFSKLKYLGTGYTPYSSNLYLCMEKEQIHARVPPELLIHLDVTTDILKSFYLLPSVIHRLQSLMLASQLRREIGYNQHIPVTLVCSLSTFLFAKDDYAFINNLVYFSCTGKPLLMEKEQIHARVPPELLIHLDILEAITTLRCCETFSLERLELLGDSVLKYVVGCDLFLRYPMKHEGQLSDMRSKAVCNATLHKHGIWRSLQGYVRDNAFDPRRWVAPGQISLRPFPCNCGIETAFVPSHRRYIRDDPSFFVGKPCDRGHRWMCSKTISDCVEALVGAYYVGGGIAAALWVMRWFGIDIKCDMKLLQEVKFNASHLCSLSKINDIEELEAKLKYNFSVKGLLLEAITHPSLQELGVDYCYQRLEFLGDSVLDLLLTRHLYATHTDVDPGELTDLRSALVSNENFAQAVVRNNIHSHLQHGSGILLEQITEYVRSNLECQGKESEFLQHTTCKVPKVLGDIMESIAGAVFIDTDFNVDMVWEIFEPLLSPLITPDKLALPPYRELLELCSHIGCFLNSKCTSKGEEVIIEMSLQLRDELLVAQGHDRNKKRAKAKAASRILADLKQQQGLSIKQCLSKAKQLDIVTSDLQFDLTSLCFHSKWRKVDLEVRFSSYARFCSGQCQNSNLWNKGSGLLLLWMGRQQQTSIALFRQSPCTYLTQQPLHFKCLERLKIRLRESTW

SEQ ID NO: 14 - DNA, 4812 bp, Oryza sativa
atggccgacgacgaggctgccgtcctcccgcccccgcctccgctgccgccgccttgccgcccccacaggcagctccgcccgagggggtctcgaccgactgctgataccacccctcgcactagccagttggtggaggtgttcgaggcggcgctgcgggggaacaccatcgcggtgctcgacacggggtccgggaagaccatggtcgccgtcatgctcgcgcgcgagcacgcacgccgggtgcgcgccggggaggcgccgcggcggatcgtggtgttcctcgcgcccaccgtgcacctcgtccatcagcaattcgaggtgattcgtgagtacactgacctcgacgtgatgatgtgctctggagcatcgcgggttggcgaatggggcgccgatcattggaaggaggaagttgggagaaatgagatcgttgttatgacgccacagatactgttggatgctctgcggcatgcttttctgacaatgagtgcagtgagcttgctaatatttgatgaatgtcatcgtgcttgtggaagccatccatatgcacgaataatgaagatatacatcgtagaagatcgaaatgagcttgagagcttttcccctcctacaacaattgtgaacaaatactatgatgcttacatggttgattttgataatctgaaatcaaagcttcagatattttctgatgagtttgattctttgttggttggtcttcaagaatcgccatctaataaatttaaagacaccgataatatcctagagacttcaagaaagagcttgtccagataccatgggaaaatattgtacagcctaaacgatcttggtccaattatcacctctgaggtagtcaaaatacatattgaaagcgttaagccattatgtgattctgaagactgcattttttctaaagctagcttgtgcttacatatgtcttattttaaagaagctttaagtctaatagaggaaattcttccacaaggatatggtgaactaatgaaatcagaatctggttctgaggaattaactaaaaggggatacatttcttcaaaagtgaatacgctaatcaacatcttcaaatcgtttgggtcatcaaatgaagtgctttgcctaattttcgtagacagaattataacagctaaagccgtcgaaaggtttatgagaggaattgttaacttctcttgtttttcaatttcttacttgactggagggagtacatcaaaagatgctctgagtccagcagttcagagatttactttggatttgttccgagctggaaaggtgaacttgctttttacaacagatgtgactgaagagggcgtcgatgtacctaactgttcttgtgtgatacgcttcgacctacccagaactgtttgtagctatgtccaatctcgtggtcgtgctagaaggaacaactcggaatttattcttatgattgagaggggaaacttgcagcagcaagaacacatatttcgtatgatacagactggttactatgttaaaaactgtgcactctatagacaccccaatgctttatcctatgacttgtctatccaagggatgtacacctaccaagttcagtcaactggagcaactataaccgcagattgctgtgtcaacctaattcgtaaatactgtgagaagcttcctaaagataggtatttcatgccaaagccttcctttgaggtgaccattgaagatggattattcaaatgcacattgacgctacctcgaaatgcagcatttcaaagtatagttggccctttaagcagttcaagtaatttatccaagcagcttgtatccctagaggcctgcaagaaattgcatcaactgggagaacttaatgatcatcttgtacctttgactgaagaacctatggatacagatttcactacagcagatgaaaaatgcatatctggaccaggaacaactaaaaggaaggagcttcatggtactacatgtgttcttgctttatcaggaacttggattcatgacagtgaaaatattacactgaatacttacagaattgattttctttgtgaccaagagggtgaaaactatgctgggtttgttctcttaatggaaccagaacttgatgatgatgtggcaccctcaaaaatggatctgttcctgatccctaataaaatggtctacaccactgtaactcctcgcggaaaagttcaactaaacaaaaagcagttaggtaaagggaaattgttccaagaattctttttcaatggaatctttggtagattatttcatggttctcgaaaaagtggagcacaaagggattttattttcaaaaagggtcatgaaatacagtggaacacggaaagcatgtacttgcttttacctttgagggattcttcatatatccaggatgacctaagcatacactgggaagcaattgaatcttgtgctggtgcagttgagcagttgtggagttcgtatcaaggagatgaaaatgtcattcctgtaaattgtattccacaaaaaagaagagggggccaagaagaaattattcatctggccaataagtctcttcattgttccagcatcaaagattcagtcgtgctatcactgcatacaggaaggatatacactgttcttgatttgatcttagacacaactgcagaggactcgtttgatgagatgtatggctcctgtgtcctgatgaactttctttcttcacttcattgtaggtatggtattattattcaacatccagaacaaccactattgctgttaaagcaaagccacaatgcacacaatcttctcttttcaaaattgaagtatctaggtactggatacacaccctactctagtaacctttacctctgtatggaaaaagaacaaattcatgctcgggttccacctgaactacttatccatctcgatgtaacaactgatattctgaagtcattttatttactcccttctgtaatacatcggcttcagtcacttatgctagccagccagcttcgcagagaaattggttacaatcaacacataccagtcactttggtttgttctctttcaacatttttatttgctaaggatgactatgcatttatcaataatttggtatatttttcatgtactggcaaacctctgctcatggaaaaagaacaaattcatgctcgggttccacctgaactacttatccatctcgatattttggaagctataacaactttaagatgctgtgagacattttctctggagcgtttagagctgttaggagactccgtgctaaagtatgtggtaggatgtgaccttttcctaaggtatcctatgaaacatgaaggtcagctctctgatatgagatccaaggctgtctgcaatgctacacttcataaacatggaatatggcggtcgttgcagggttatgtacgtgataatgcttttgacccacggcgttgggttgctcctggacagatatcgttgcgcccttttccttgtaactgtggaatcgagactgcatttgttccttctcatagaaggtatatccgagatgacccatctttttttgtgggaaaaccatgtgacagaggtcataggtggatgtgctcaaaaacaatatctgattgtgttgaagcactggttggagcatattatgttggtggtggcattgctgctgcactttgggttatgaggtggtttggaatcgatatcaaatgtgatatgaagctattgcaggaagtgaagttcaatgcatctcatttatgctccttatcaaaaataaatgacattgaggaactggaagcaaaactgaagtacaacttctcagtcaagggccttcttttggaagccataactcatccatctctgcaggaattaggtgttgattactgttaccagcgtcttgaatttcttggtgattctgtgctggatctacttcttacacgtcatctctatgctactcatactgatgttgatcctggagaattaacggatttacgctccgctttggttagtaatgagaattttgcacaagcagttgtaagaaacaacattcacagtcatctacaacatggatccggaatacttttggagcaaattactgaatatgtcaggtcaaatttggagtgtcaagggaaagagagtgaattccttcaacatactacatgtaaagtacctaaggttcttggtgacattatggaaagcatcgctggtgcagtatttatagacaccgattttaatgttgacatggtttgggagattttcgagccattgctttctccactgattacacctgataagcttgcattgccaccttaccgtgagttgctggagctatgcagtcacattggttgcttcttaaattcaaaatgcaccagtaaaggagaagaagtaattatagagatgtcactgcaactacgagatgagctgctggtagcacaagggcatgacagaaacaaaaagagggcaaaggcaaaagcagcatctcgtattttggcagatcttaagcagcaacagggtctttcaattaaacaatgtttgtccaaggctaaacagctggatatcgtgacttcagatcttcagtttgacttgacaagtttgtgcttccactcaaaatggagaaaggtggacctcgaagtgcgcttttcaagctatgcaagattttgcagtggccaatgccagaattcgaatttgtggaacaaaggttcaggactcctattgttatggatggggcgacaacaacaaacttcaatagctttgtttcgacaatcaccttgcacatacctgacgcaacaaccattacatttcaagtgtttggagagattaaagatacgtcttagagaaagcacttggtag

SEQ ID NO: 15 - DNA, 21 bp, Artificial
  oligonucleotide primer for the amplification of fragment 1 of the coding sequence of DCL3
atgcattcgtcgttggagccg

SEQ ID NO: 16 - DNA, 21 bp, Artificial
  oligonucleotide primer for the amplification of fragment 1 of the coding sequence of DCL3
ttagaatctgggataaccatg

SEQ ID NO: 17 - DNA, 21 bp, Artificial
  oligonucleotide primer for the amplification of fragment 2 of the coding sequence of DCL3
ctccagtgaaaaatggacacg

SEQ ID NO: 18 - DNA, 21 bp, Artificial
  oligonucleotide primer for the amplification of fragment 2 of thecoding sequence of DCL3
tcttcacaaccatctcttcat

SEQ ID NO: 19 - DNA, 21 bp, Artificial
  oligonucleotide primer for the amplification of fragment 3 of thecoding sequence of DCL3
aagcagagtcaccatgcgcac

SEQ ID NO: 20 - DNA, 21 bp, Artificial
  oligonucleotide primer for the amplification of fragment 3 of the coding sequence of DCL3
ctccacaacatctccaagagc

SEQ ID NO: 21 - DNA, 21 bp, Artificial
  oligonucleotide primer for the amplification of fragment 4 of thecoding sequence of DCL3
tgagactggtagatcaatccc

SEQ ID NO: 22 - DNA, 31 bp, Artificial
  oligonucleotide primer for the amplification of fragment 4 of the coding sequence of DCL3
cgtctcttttttttttttttttttttttttt

SEQ ID NO: 23 - DNA, 21 bp, Artificial
  oligonucleotide primer for the amplification of fragment 1 of thecoding sequence of DCL4
atgcgtgacgaagttgacttg

SEQ ID NO: 24 - DNA, 21 bp, Artificial
  oligonucleotide primer for the amplification of fragment 1 of the coding sequence of DCL4
agacagcatgtctgaatatca

SEQ ID NO: 25 - DNA, 21 bp, Artificial
  oligonucleotide primer for the amplification of fragment 2 of the coding sequence of DCL4
aaagttggtgaagaaggcctt

SEQ ID NO: 26 - DNA, 21 bp, Artificial
  oligonucleotide primer for the amplification of fragment 2 of the coding sequence of DCL4
cttacagatctttgatgagca

SEQ ID NO: 27 - DNA, 21 bp, Artificial
  oligonucleotide primer for the amplification of fragment 3 of the coding sequence of DCL4
gctgagactatggatatcgat

SEQ ID NO: 28 - DNA, 21 bp, Artificial
  oligonucleotide primer for the amplification of fragment 3 of the coding sequence of DCL4
gctcatgacatttctctgttg

SEQ ID NO: 29 - DNA, 21 bp, Artificial
  oligonucleotide primer for the amplification of fragment 4 of the coding sequence of DCL4
gtttctggtcacagggtactc

SEQ ID NO: 30 - DNA, 21 bp, Artificial
  oligonucleotide primer for the amplification of fragment 4 of the coding sequence of DCL4
gcaaaggaatccagaatgctt

SEQ ID NO: 31 - DNA, 20 bp, Artificial
  forward oligonucleotide primer for diagnostic PCR amplificationof DCL3
ggcttcaagagttgggaaaa

SEQ ID NO: 32 - DNA, 20 bp, Artificial
  reverse oligonucleotide primer for diagnostic PCR amplificationof DCL3.
cttgcacaccattgagcatt

SEQ ID NO: 33 - DNA, 28 bp, Artificial
  forward oligonucleotide primer for diagnostic PCR amplificationof DCL4.
gcaggttcttggtgacttggtagaatcc

SEQ ID NO: 34 - DNA, 26 bp, Artificial
  reverse oligonucleotide primer for diagnostic PCR amplificationof DCL4
caggtggcctggtccttcctcttcac

SEQ ID NO: 35 - DNA, 30 bp, Artificial
  forward oligonucleotide primer for diagnostic PCR amplificationof DCL3A
tcttttctwactggagggagwtcttcrgtg

SEQ ID NO: 36 - DNA, 25 bp, Artificial
  reverse oligonucleotide primer for diagnostic PCR amplificationof DCL3A
acttctcayaatyscagatatcaaa

SEQ ID NO: 37 - DNA, 30 bp, Artificial
  forward oligonucleotide primer for diagnostic PCR amplificationof DCL3B
tcatacttgactggagggagtacatcaaaa

SEQ ID NO: 38 - DNA, 26 bp, Artificial
  reverse oligonucleotide primer for diagnostic PCR amplificationof DCL3B
gtatyatacgaaatatgtgttcytgc